(19) United States
(12) Patent Application Publication
(10) Pub. No.: US 2017/0135633 A1
(43) Pub. Date: May 18, 2017

(54) Integrated System for Managing Cardiac Rhythm Including Wearable and Implanted Devices

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 15/418,620

(22) Filed: Jan. 27, 2017

Related U.S. Application Data
(63) Continuation-in-part of application No. 14/951,475, filed on Nov. 24, 2015.
Application No. 14/951,475 is a continuation-in-part of application No. 13/901,131, filed on May 23, 2013, now Pat. No. 9,536,449.
Application No. 13/901,131 is a continuation-in-part of application No. 14/071,112, filed on Nov. 4, 2013, now abandoned.
Application No. 14/071,112 is a continuation-in-part of application No. 14/623,337, filed on Feb. 16, 2015, now Pat. No. 9,582,035.
(60) Provisional application No. 62/245,311, filed on Oct. 23, 2015.
(60) Provisional application No. 62/297,827, filed on Feb. 20, 2016.
(60) Provisional application No. 62/439,147, filed on Dec. 26, 2016.

(51) Int. Cl. A61B 5/00; A61N 1/372; A61N 1/365; A61B 5/1455; A61B 5/145

(52) U.S. Cl. CPC A61B 5/4836; A61B 5/6826; A61B 5/681; A61B 5/6824; A61B 5/6816; A61B 5/14552; A61B 5/14546; A61N 1/36557; A61N 1/37217

(57) ABSTRACT

This invention is an integrated system for managing cardiac rhythm comprising a wearable device (such as a finger wring or wrist band) that measures body oxygen levels and an implanted cardiac rhythm management device (such as a pacemaker). Working together in an integrated system, a wearable device for measuring oxygen level in body extremities and an implanted device for cardiac rhythm management can help to prevent oxygen deficiencies in body extremities.

### CROSS-REFERENCE TO RELATED APPLICATIONS:

This patent application: (1) is a continuation in part of U.S. patent application Ser. No. 14/951,475 by Robert A. Connor entitled “Wearable Spectroscopic Sensor to Measure Food Consumption Based on Interaction Between Light and the Human Body” filed on Nov. 24, 2015 which, in turn: (a) is a continuation in part of U.S. patent application Ser. No. 13/901,131 by Robert A. Connor entitled “Smart Watch and Food Utensil for Monitoring Food Consumption” filed on May 23, 2013; (b) is a continuation in part of U.S. patent application Ser. No. 14/071,112 by Robert A. Connor entitled “Wearable Spectroscopy Sensor to Measure Food Consumption” filed on Nov. 4, 2013; (c) is a continuation in part of U.S. patent application Ser. No. 14/623,337 by Robert A. Connor entitled “Wearable Computing Devices and Methods for the Wrist and/or Forearm” filed on Feb. 16, 2015; and (d) claims the priority benefit of U.S. provisional patent application 62/245,311 by Robert A. Connor entitled “Wearable Device for the Arm with Close-Fitting Biometric Sensors” filed on Oct. 23, 2015; (2) claims the priority benefit of U.S. provisional patent application 62/297,827 by Robert A. Connor entitled “System for Automatic Adjustment of Cardiac Function Based on Data from a Wearable Biometric Sensor” filed on Feb. 20, 2016; and (3) claims the priority benefit of U.S. provisional patent application 62/439,147 by Robert A. Connor entitled “Arcuate Wearable Device for Measuring Body Hydration and/or Glucose Level” filed on Dec. 26, 2016. The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to cardiac rhythm management.

### INTRODUCTION

Proper blood circulation and oxygenation for tissue in body extremities is important for physiological functioning and tissue health. Various factors, including exercise, can change oxygen levels in body extremities. It would be desirable to have an implanted cardiac rhythm management device be aware of low oxygen levels in body extremities and to respond with increased blood circulation. This can help to ensure good physiological functioning, improve extremity tissue health, and potentially even avoid long-term limb loss due to poor circulation and low tissue oxygenation. This is the unmet clinical need which is addressed by this invention.

### REVIEW OF THE PRIOR ART

U.S. Patent Applications 20050115561 (Stahmann et al., Jun. 2, 2005, “Patient Monitoring, Diagnosis, and/or Therapy Systems and Methods”) and 20110061647 (Stahmann et al., Mar. 17, 2011, “Patient Monitoring, Diagnosis, and/or Therapy Systems and Methods”) and U.S. Pat. No. 7,787,946 (Stahmann et al., Aug. 31, 2010, “Patient Monitoring, Diagnosis, and/or Therapy Systems and Methods”) disclose cooperative communication between an implantable cardiac function device and an external respiratory therapy device. U.S. Pat. No. 8,515,548 (Rofougaran et al., Aug. 20, 2013, “Article of Clothing Including Bio-Medical Units”) discloses clothing with a plurality of bio-medical units for physical therapy. U.S. Patent Application 20060195039 (Drew et al., Aug. 31, 2006, “Clustering with Combined Physiological Signals”) and U.S. Pat. No. 8,768,446 (Drew et al., Jul. 1, 2014, “Clustering with Combined Physiological Signals”) disclose the generation of an extended cluster of data for activation of implantable systems such as those that provide stimulation and drug delivery, pacemaker systems, defibrillator systems, and cochlear implant systems. U.S. Patent Application 20160018347 (Drbal et al., Jan. 21, 2016, “Designs, Systems, Configurations, and Methods for Immittance Spectroscopy”) discloses the use of immittance spectroscopy to identify the composition of liquids. U.S. Patent Application 20140316479 (Taff et al., Oct. 23, 2014, “Implantable Medical Device”) discloses a leadless pacemaker which may include a spectroscopic sensor. U.S. Pat. No. 8,463,345 (Kuhn et al., Jun. 11, 2013, “Device and Method for Monitoring of Absolute Oxygen Saturation and Total Hemoglobin Concentration”), U.S. Pat. No. 8,634,890 (Kuhn et al., Jan. 21, 2014, “Device and Method for Monitoring of Absolute Oxygen Saturation and Tissue Hemoglobin Concentration”), and U.S. Pat. No. 8,666,466 (Kuhn et al., Mar. 4, 2014, “Device and Method for Monitoring of Absolute Oxygen Saturation and Tissue Hemoglobin Concentration”) disclose an implanted oxygen saturation monitor. U.S. Pat. No. 8,428,729 (Schwartz et al., Apr. 23, 2013, “Cardiac Stimulation Apparatus and Method for the Control of Hypertension”) discloses changing cardiac rhythm based on changes in blood pressure.

U.S. Pat. No. 8,112,148 (Giftakis et al., Feb. 7, 2012, “System and Method for Monitoring Cardiac Signal Activity in Patients with Nervous System Disorders”) discloses the use of brain event information to interpret cardiac signals. U.S. Patent Application 20040131998 (Marom et al., Jul. 8, 2004, “Cerebral Programming”) and U.S. Pat. No. 7,499,894 (Marom et al., Mar. 3, 2009, “Cerebral Programming”) disclose training a biological neural network to control an insulin pump or a pacemaker. U.S. Patent Applications 20050081847 (Lee et al., Apr. 21, 2005, “Automatic Activation of Medical Processes”) and 20100106211 (Lee et al., Apr. 29, 2010, “Automatic Activation of Medical Processes”) and U.S. Pat. No. 7,668,591 (Lee et al., Feb. 23, 2010, “Automatic Activation of Medical Processes”), U.S. Pat. No. 7,668,591 (Lee et al., Feb. 23, 2010, “Automatic Activation of Medical Processes”), and U.S. Pat. No. 8,380,296 (Lee et al., Feb. 19, 2013, “Automatic Activation of Medical Processes”) disclose changing cardiac rhythm therapy based on brain state information. U.S. Patent Applications 20070260286 (Giftakis et al., Nov. 8, 2007, “System and Method for Utilizing Brain State Information to Modulate Cardiac Therapy”) and 20070265677 (Giftakis et al., Nov. 15, 2007, “System and Method for Utilizing Brain State Information to Modulate Cardiac Therapy”) and U.S. Pat. No. 8,209,019 (Giftakis et al., Jun. 26, 2012, “System and Method for Utilizing Brain State Information to Modulate Cardiac Therapy”) and U.S. Pat. No. 8,214,035 (Giftakis et al., Jul. 3, 2012, “System and Method for Utilizing Brain State Information to Modulate Cardiac Therapy”) disclose changing cardiac therapy based on brain state information.

### SUMMARY OF THE INVENTION

This invention can be embodied in an integrated system for managing cardiac rhythm including a wearable device that measures body oxygen levels and an implanted cardiac rhythm management device. The synergistic integration of the wearable device and the implanted cardiac rhythm management device can enable cardiac rhythm management that is superior to that provided by either component alone. For example, without an implanted cardiac rhythm management device, a wearable device alone can provide information on oxygenation levels in body extremities, but does not provide automatic therapeutic correction for oxygenation deficiency in body extremities. Similarly, without a wearable device component to measure body oxygen levels in body extremities, an implanted cardiac rhythm management device alone is not aware of oxygen deficiencies in body extremities. Working together in an integrated system, a wearable device for measuring oxygen level in body extremities and an implanted device for cardiac rhythm management can help to prevent oxygen deficiencies in body extremities. This can help to avoid physiological dysfunction and potentially even limb loss due to poor circulation and oxygenation.

This invention can be embodied in an integrated system for managing cardiac rhythm including both a wearable device and an implanted device, wherein this system comprises: (a) a wearable device which is configured to be worn by a person, wherein the wearable device further comprises a light emitter which is configured to emit light toward the person's body tissue, a light receiver which is configured to receive light from the light emitter after the light has passed through and/or been reflected from the person's body tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's body tissue; and (d) wherein the operation of the cardiac rhythm management device is changed based on the amount of the analyte in the person's body tissue. In an example, the wearable component of the system can be a finger ring and the measured analyte can be oxygen level. Body tissue herein is understood to include blood, interstitial fluid, and other body fluids.

### INTRODUCTION TO THE FIGURES

FIG. 1 shows a system comprising a wrist band with a biosensor in communication with an implanted cardiac rhythm management device.

FIG. 2 shows a system comprising a finger ring with a biosensor in communication with an implanted cardiac rhythm management device.

FIG. 3 shows a close-up view of the finger ring introduced in FIG. 2.

FIG. 4 shows a system comprising an ear-worn device with a biosensor in communication with an implanted cardiac rhythm management device.

FIG. 5 shows a close-up view of the ear-worn device introduced in FIG. 4.

FIG. 6 shows a system comprising a finger ring with a light emitter and a light receiver in communication with an implanted cardiac rhythm management device.

FIG. 7 shows a wearable device with spectroscopic sensors at different locations on the device circumference.

FIG. 8 shows a wearable device with spectroscopic sensors with different light-projection angles.

FIG. 9 shows a wearable device with a rotating spectroscopic sensor.

FIG. 10 shows a wearable device with a two-dimensional array of spectroscopic sensors.

FIG. 11 shows a wearable device with spectroscopic sensors pushed inward by hydraulic, pneumatic, or electromagnetic mechanisms.

FIG. 12 shows a wearable device with spectroscopic sensors pushed inward by individual springs.

FIG. 13 shows a wearable device with spectroscopic sensors on an inward-facing surface connected to an elastic compartment.

FIG. 14 shows a wearable device with spectroscopic sensors on an inward-facing surface that pivots around a joint.

FIG. 15 shows a wearable device with spectroscopic sensors on an inward-facing surface pressed inward by springs.

FIG. 16 shows a wearable device with a spectroscopic sensor on an elastic compartment.

FIG. 17 shows a wearable device with multiple spectroscopic sensors on an elastic compartment.

FIG. 18 shows a wearable device with a spectroscopic sensor on an elastic compartment with adjustable pressure.

FIG. 19 shows a wearable device with spectroscopic sensors on elastic compartments on a strap or band.

FIG. 20 shows a wearable device with spectroscopic sensors on toroidal elastic compartments.

FIG. 21 shows a wearable device with spectroscopic sensors on interconnected toroidal elastic compartments.

FIG. 22 shows a wearable device with a spectroscopic sensor on a rotating ball.

FIG. 23 shows a wearable device with an undulating band with spectroscopic sensors.

FIG. 24 shows a wearable device with an undulating band with six undulations and spectroscopic sensors.

FIG. 25 shows a wearable device with a laterally-undulating band with spectroscopic sensors.

FIG. 26 shows a wearable device with one or more elastic portions which are configured to span the anterior (upper) surface of a person's arm, one or more inelastic portions which are configured to span the posterior (lower) surface of the person's arm, an enclosure which is connected to the elastic portions, and one or more spectroscopic sensors which are part of the enclosure.

FIG. 27 shows a wearable device with one or more anterior inelastic portions which are configured to span the anterior (upper) surface of a person's arm, one or more posterior inelastic portions which are configured to span the posterior (lower) surface of a person's arm, one or more elastic portions which connect the anterior and posterior inelastic portions, an enclosure which is configured to be worn on the anterior (upper) portion of the arm, and one or more spectroscopic sensors which are part of the enclosure.

FIG. 28 shows a wearable device with a relatively-rigid band and a relatively-elastic band, wherein each of these bands spans at least 60% of the circumference of a person's arm, wherein these bands are connected to each other, and wherein there are spectroscopic sensors on the relatively-elastic band.

FIG. 29 shows a wearable device with two or more modular and connectable bands, wherein each band spans at least 60% of the circumference of a person's arm, and wherein one or more of these bands house spectroscopic sensors.

FIG. 30 shows a wearable device with a partial-circumferential inner elastic band and spectroscopic sensors.

FIG. 31 shows a wearable device wherein an outer inelastic band is sufficiently resilient that its ends hold onto the person's arm without the need for a clasp.

FIG. 32 shows a wearable device with an outer arcuate inelastic band, an inner arcuate elastic band, and spectroscopic sensors which are part of the inner band.

FIG. 33 shows a wearable device with an outer rigid “clam shell” structure to hold a display screen in place and an inner arcuate elastic band to keep spectroscopic sensors close against the surface of the arm.

FIG. 34 shows a wearable device with an inner arcuate elastic band which spans the posterior (lower) surface of a person's arm.

FIG. 35 shows a wearable device with an outer rigid “clam shell” structure and inward-facing flexible undulations to keep spectroscopic sensors close against the surface of the arm.

FIG. 36 shows a wearable device with two display screens suspended by an elastic material between two arcuate bands.

### DETAILED DESCRIPTION OF THE FIGURES

An introductory section is now provided herein, before detailed discussion of specific figures and examples. Example variations discussed in this introductory section can be applied where relevant to each of the specific figures and examples which follow, but this material is not repeated in each the narratives accompanying each individual figure and example in order to avoid redundancy in the disclosure.

In an example, this invention can be embodied in a system (or device) for automatic adjustment of an implanted cardiac management device comprising: a wearable component which is configured to be worn on a person's body or clothing; a biometric sensor which is configured to be held in proximity to the surface of the person's body by the wearable component; a data processor which receives data from the biometric sensor; and an implanted cardiac management device which is configured to manage (or control or change) the person's cardiac function, wherein the operation of the implanted cardiac management device is automatically adjusted based on analysis of data from the biometric sensor. In an example, being in proximity to the surface of the person's body can be defined as having at least one part which is worn less than three inches away from the person's body.

In an example, this invention can be embodied in a system (or device) for automatic adjustment of an implanted cardiac management device comprising: a wearable component which is configured to be worn on a person's body or clothing; a biometric sensor which is configured to be held in proximity to the surface of the person's body by the wearable component; a data processor which receives data from the biometric sensor; and an implanted cardiac management device which is configured to manage (or control or change) the functioning of the person's heart, wherein the operation of the implanted cardiac management device is automatically adjusted based on analysis of data from the biometric sensor. In an example, being in proximity to the surface of the person's body can be defined as having at least one part which is worn less than three inches away from the person's body.

In an example, this invention can be embodied in a system (or device) for automatic adjustment of an implanted cardiac management device comprising: a wearable component which is configured to be worn on a person's body or clothing; at least one spectroscopic sensor which is configured to be held in proximity to the surface of the person's body by the wearable component; a data processor which receives data from the biometric sensor; and an implanted cardiac management device which is configured to manage (or control or change) the person's cardiac function, wherein the operation of the implanted cardiac management device is automatically adjusted based on analysis of data from the biometric sensor. In an example, being in proximity to the surface of the person's body can be defined as having at least one part which is worn less than three inches away from the person's body.

In an example, this invention can be embodied in a system (or device) for automatic adjustment of an implanted cardiac management device comprising: a wearable component which is configured to be worn on a person's body or clothing; at least one electroencephalographic (EEG) sensor which is configured to be held in proximity to the surface of the person's body by the wearable component; a data processor which receives data from the biometric sensor; and an implanted cardiac management device which is configured to manage (or control or change) the person's cardiac function, wherein the operation of the implanted cardiac management device is automatically adjusted based on analysis of data from the biometric sensor. In an example, being in proximity to the surface of the person's body can be defined as having at least one part which is worn less than three inches away from the person's body.

In an example, this invention can be embodied in a system (or device) for automatic adjustment of an implanted cardiac management device comprising: a wearable component which is configured to be worn on a person's body or clothing; at least one electromyographic (EMG) sensor which is configured to be held in proximity to the surface of the person's body by the wearable component; a data processor which receives data from the biometric sensor; and an implanted cardiac management device which is configured to manage (or control or change) the person's cardiac function, wherein the operation of the implanted cardiac management device is automatically adjusted based on analysis of data from the biometric sensor. In an example, being in proximity to the surface of the person's body can be defined as having at least one part which is worn less than three inches away from the person's body.

In an example, the wearable component of this system can be configured to be worn on a person's arm. Portions of a person's arm include the person's fingers, hand, wrist, forearm, elbow, and upper arm. In an example, the wearable component of this system can be worn on a person's finger and/or hand. In an example, the wearable component of this system can be selected from the group consisting of a finger ring, finger sleeve, artificial finger nail, finger nail attachment, finger tip (thimble), and glove. In an example, the wearable component of this system can be worn in a manner similar to a finger ring, finger sleeve, artificial finger nail, finger nail attachment, finger tip (thimble), or glove. In an example, a biometric sensor of this system can be integrated into a finger ring, finger sleeve, artificial finger nail, finger nail attachment, finger tip (thimble), or glove.

In an example, the wearable component of this system can be worn on a person's wrist and/or forearm. In an example, the wearable component of this system can be selected from the group consisting of an armlet, bangle, bracelet, cuff, fitness band, gauntlet, sleeve, smart watch, strap, watch, and wrist band. In an example, the wearable component of this system can be worn in a manner similar to an armlet, bangle, bracelet, cuff, fitness band, gauntlet, sleeve, smart watch, strap, watch, or wrist band. In an example, a biometric sensor of this system can be integrated into an armlet, bangle, bracelet, cuff, fitness band, gauntlet, sleeve, smart watch, strap, watch, or wrist band.

In an example, the wearable component of this system can be worn on a person's elbow, upper arm, and/or shoulder. In an example, the wearable component of this system can be an armband, compression joint sleeve, full-sleeve, or shirt. In an example, the wearable component of this system can be worn in a manner similar to an armband, compression joint sleeve, full-sleeve, or shirt. In an example, a biometric sensor of this system can be integrated into an armband, compression joint sleeve, full-sleeve, or shirt.

In an example, the wearable component of this system can have two flexible straps, bands, sides, or ends which are placed around a person's wrist and/or arm and then removably-fastened together around the wrist and/or arm by an attachment mechanism selected from the group consisting of: buckle, button, clasp, clip, hook, hook-and-eye mechanism, magnet, pin, plug, prong, and snap. In an example, the wearable component of this system can have two flexibly resilient prongs, clasps, bands, sides, or ends which are flexible enough to be pulled apart from each other by an external force in order to slip the component onto a person's wrist and/or arm but are also resilient enough to retract back towards each other and hold the wearable component around the person's wrist and/or arm when the external force is removed. In an example, the wearable component of this system can be sufficiently elastic, stretchable, and/or expandable that it can slide over a person's hand onto their wrist and/or arm.

In an example, the wearable component of this system can be configured to be worn on, around, or within a person's ear. In an example, the wearable component can be inserted (partially or fully) into the ear canal, attached to the earlobe, worn around a portion of the outer ear, or a combination thereof. In an example, an ear-worn wearable component of this system can also include a prong, arm, or other protrusion which extends forward onto the person's temple and/or their forehead. In an example, the wearable component of this system can be a “hearable” device. In an example, the wearable component of this system can be selected from the group consisting of: ear bud, ear hook, ear plug, ear ring, earlobe clip, earphone, earpiece, earring, ear-worn Bluetooth communication device, electroencephalographic (EEG) sensor, oximeter, headphone, headset, and hearing aid. In an example, the wearable component of this system can be worn in a manner similar to an ear bud, ear hook, ear plug, ear ring, earlobe clip, earphone, earpiece, earring, ear-worn Bluetooth communication device, electroencephalographic (EEG) sensor, oximeter, headphone, headset, or hearing aid. In an example, a biometric sensor of this system can be integrated into an ear bud, ear hook, ear plug, ear ring, earlobe clip, earphone, earpiece, earring, ear-worn Bluetooth communication device, electroencephalographic (EEG) sensor, oximeter, headphone, headset, or hearing aid.

In an example, the wearable component of this system can be configured to be worn on, over, and/or near one or both of a person's eyes. In an example, the wearable component of this system can be selected from the group consisting of: Augmented Reality (AR) eyewear, contact lens, electronically-functional eyewear, eyeglasses, goggles, monocle, and Virtual Reality (VR) eyewear. In an example, the wearable component of this system can be worn in a manner similar to Augmented Reality (AR) eyewear, contact lens, electronically-functional eyewear, eyeglasses, goggles, monocle, or Virtual Reality (VR) eyewear. In an example, a biometric sensor of this system can be integrated into Augmented Reality (AR) eyewear, contact lens, electronically-functional eyewear, eyeglasses, goggles, monocle, or Virtual Reality (VR) eyewear.

In an example, the wearable component of this system can be configured to be worn elsewhere on a person's head. In an example, the wearable component of this system can be selected from the group consisting of: baseball cap, face mask, hair band, hair clip, hair comb, hair pin, hat, headband, head-encircling EEG sensor band, headphones, headset, helmet, nose plug, nose ring, respiratory mask, skull cap, tiara, and visor. In an example, the wearable component of this system can be worn in a manner similar to a baseball cap, face mask, hair band, hair clip, hair comb, hair pin, hat, headband, head-encircling EEG sensor band, headphones, headset, helmet, nose plug, nose ring, respiratory mask, skull cap, tiara, or visor. In an example, a biometric sensor of this system can be integrated into a baseball cap, face mask, hair band, hair clip, hair comb, hair pin, hat, headband, head-encircling EEG sensor band, headphones, headset, helmet, nose plug, nose ring, respiratory mask, skull cap, tiara, or visor.

In an example, the wearable component of this system can be configured to be worn on a person's torso. In an example, it can be worn on and/or around a person's chest or waist. In an example, it can be worn in a manner similar to a shirt, undershirt, bra, belt, collar, jacket, necklace, chest strap, waist band, waist strap, or compression belt. In an example, it can be worn on and/or around a person's chest or waist. In an example, it can be a shirt, undershirt, bra, belt, collar, jacket, necklace, chest strap, waist band, waist strap, or compression belt. In an example, a biometric sensor of this system can be integrated into a shirt, undershirt, bra, belt, collar, jacket, necklace, chest strap, waist band, waist strap, or compression belt.

In an example, the wearable component of this system can be configured to be worn on a person's leg and/or foot. In an example, it can be worn in a manner similar to a sock, shoe, leg band, knee brace, pants, underpants, jumpsuit, or shorts. In an example, it can be a sock, shoe, leg band, knee brace, pair of pants, underpants, jumpsuit, or pair of shorts. In an example, a biometric sensor of this system can be integrated into a sock, shoe, leg band, knee brace, pair of pants, underpants, jumpsuit, or pair of shorts.

In an example, this system can include a biometric sensor which is part of the wearable component of this system. In an example, a biometric sensor collects data concerning a biological or physiological parameter or condition concerning the body of the person wearing the wearable component. In an example, a biometric sensor can be in direct physical contact with the surface of a person's body. In an example, a biometric sensor can be in direct physical contact with the person's skin. In an example, a biometric sensor can be in fluid and/or gaseous communication with body tissue, organs, and/or fluid. In an example, a biometric sensor can be in optical communication with body tissue, organs, and/or fluid. In an example, a biometric sensor can be in electromagnetic communication with body tissue, organs, and/or fluid. In an example, a biometric sensor can be in electromagnetic communication with body tissue, organs, and/or fluid through a layer of clothing.

In an example, the system (or device) can include a plurality of biometric sensors. In an example, one or more biometric sensors can be housed in a wearable component. In an example, one or more biometric sensors can be held by a wearable component. In an example, this system can include a plurality of biometric sensors at different locations relative to the wearable component and/or at different locations relative to a person's body.

In an example, the wearable component can comprise a plurality of biometric sensors which are configured to measure energy which is reflected from (or passed through) the person's body at different angles. In an example, the wearable component can comprise a plurality of biometric sensors which are configured to measure energy which is naturally emitted by the person's body. In an example, the wearable component can comprise a plurality of biometric sensors which are configured to measure energy which is reflected from (or passed through) the person's body at different wavelengths. In an example, the wearable component can comprise a plurality of biometric sensors which are configured to measure energy which is naturally emitted by the person's body at different wavelengths.

In an example, a biometric sensor can be a light sensor (which can alternatively be called an “optical sensor” or “optical detector” or “spectroscopic sensor” or “spectroscopy sensor”) which receives light energy which has been reflected from, or passed through, body tissue, organs, and/or fluid. In an example, this light sensor can be a spectroscopic sensor (which can alternatively be called a “spectroscopy sensor”). A spectroscopic sensor collects data concerning the spectrum of light energy which has been reflected from (or has passed through) body tissue, organs, and/or fluid. This data concerning light energy is used to analyze the spectral distribution of that light and thereby infer the chemical composition and/or physical configuration of the body tissue, organs, and/or fluid.

In an example, a spectroscopic sensor can be selected from the group consisting of: ambient light spectroscopic sensor, analytical chromatographic sensor, backscattering spectrometry sensor, spectroscopic camera, chemiluminescence sensor, chromatographic sensor, coherent light spectroscopic sensor, colorimetric sensor, fiber optic spectroscopic sensor, fluorescence sensor, gas chromatography sensor, infrared light sensor, infrared spectroscopic sensor, ion mobility spectroscopic sensor, laser spectroscopic sensor, liquid chromatography sensor, mass spectrometry sensor, near infrared spectroscopic sensor, optoelectronic sensor, photocell, photochemical sensor, Raman spectroscopy sensor, spectral analysis sensor, spectrographic sensor, spectrometric sensor, spectrometry sensor, spectrophotometer, spectroscopic glucose sensor, spectroscopic oximeter, ultraviolet light sensor, ultraviolet spectroscopic sensor, variable focal-length camera, video camera, visible light spectroscopic sensor, and white light spectroscopic sensor.

In an example, a spectroscopic sensor can comprise a light receiver alone if it receives ambient light which has been reflected from (or has passed through) body tissue, organs, and/or fluid. In an example, a spectroscopic sensor can comprise both a light emitter and a light receiver if it the light receiver receives light which has been emitted by the light emitter and then reflected from (or passed through) body tissue, organs, and/or fluid. In an example, a light emitter and light receiver can be paired together. In an example, a light emitter and light receiver together can be referred to as a spectroscopic sensor.

In an example, a biometric sensor of this system can be a spectroscopic sensor, including a light emitter and light receiver, which collects light energy data which then is analyzed using spectroscopic analysis in order to measure the chemical composition of body tissue, organs, and/or fluid. In an example, a biometric sensor of this system can be a spectroscopic sensor, including a light emitter and light receiver, which collects light energy data which then is analyzed using spectroscopic analysis in order to monitor changes in the chemical composition of body tissue, organs, and/or fluid. In an example, changes, gaps, and/or shifts in selected frequencies in the spectrum of ambient light due to interaction with a person's body tissue and/or fluid can be analyzed to monitor changes in the chemical composition of the person's body tissue and/or fluid. In an example, data from a spectroscopic sensor can be analyzed to determine how the spectrum of ambient light has been changed by reflection from, or passage through, body tissue, organs, and/or fluid.

In an example, the biometric sensor of this system can be a spectroscopic sensor, including a light emitter and light receiver, which collects light energy data which then is analyzed using spectroscopic analysis in order to measure the physical configuration of body tissue, organs, and/or fluid. In an example, the biometric sensor of this system can be a spectroscopic sensor, including a light emitter and light receiver, which collects light energy data which then is analyzed using spectroscopic analysis in order to monitor changes in the physical configuration of body tissue, organs, and/or fluid.

In an example, a spectroscopic sensor of this system can include one or more light (energy) emitters. In an example, one or more light (energy) emitters can be selected from the following types of light emitters: arc source, blackbody source, coherent light source, incandescent bulb, infrared light emitter, laser, Laser Diode (LD), Light Emitting Diode (LED), mercury lamp, microplasma light emitter, multi-wavelength source, Organic Light Emitting Diode (OLED), Resonant Cavity Light Emitting Diode (RCLED), Superluminescent Light Emitting Diode (SLED), ultraviolet light emitter, and tungsten lamp.

In an example, a spectroscopic sensor of this system can include one or more light emitters which emit light energy toward a person's skin and/or body surface. In an example, one or more light emitters can emit light energy toward a person's body tissue, organs, and/or fluid. In an example, one or more light emitters can deliver light energy to a person's body tissue, organs, and/or fluid. In an example, one or more light emitters can deliver light energy to body tissue, organs, and/or fluid directly via direct optical communication. In an example, one or more light emitters can deliver light energy to body tissue, organs, and/or fluid indirectly via one or more light guides. In an example, this light energy can be reflected from body tissue, organs, and/or fluid and then the reflected light energy can be received by a light receiver, which is also part of this system. In an example, this light energy can be transmitted through body tissue, organs, and/or fluid and then the transmitted light energy can be received by a light receiver, which is also part of this system. In an example, one or more light emitters can deliver light energy in one or more selected wavelengths (or wavelength ranges or spectra) to body tissue, organs, and/or fluid. In an example, one or more light emitters can deliver infrared light energy, near infrared light energy, ultraviolet light energy, and/or visible light energy to body tissue, organs, and/or fluid.

In an example, the wearable component of this system can comprise a light-emitting member (such as an LED) which is configured to direct light toward the person's body. In an example, this light can be infrared light, near-infrared light, ultraviolet light, and visible and/or white light. In an example, this light can be coherent and/or laser light. In an example, a spectroscopic sensor can receive this directed light after it has been reflected from, or passed through, the person's body tissue and/or fluid. In an example, data from a spectroscopic sensor can be analyzed to determine how the spectrum of directed light has been changed by reflection from, or passage through, the person's body tissue and/or fluid. In an example, changes in the spectrum of directed light due to interaction with a person's body tissue and/or fluid can be analyzed to measure (changes in) the chemical composition of the person's body tissue and/or fluid.

In an example, this system can include one or more light guides which direct light energy from a first location, angle, and/or transmission vector to a second location, angle, and/or transmission vector. In an example, a light guide can direct light from a light emitter toward body tissue, organs, and/or fluid. In an example, a light guide can collect and direct ambient light toward body tissue, organs, and/or fluid. In an example, a light guide can direct light reflected from, or having passed through, body tissue, organs, and/or fluid toward a light receiver. In an example, a light guide can be generally cylindrical and/or columnar. In an example, a light guide can be rigid. In an example, a light guide can be flexible. In an example, a light guide can have a refractive index of at least 3.141. In an example, a light guide can be made from one or more materials selected from the group consisting of: acrylic, crystal, elastomeric light-transmissive material, glass, high-durometer plastic, low-durometer plastic, optical-pass material, polycarbonate, polyethylene, polymer, polyurethane, resin, sapphire, and transparent polymer.

In an example, the wearable component of this system can include one or more light filters. In an example, a light filter can partially absorb and/or block light transmission between a light emitter and body tissue. In an example, a light filter can partially absorb and/or block light transmission between ambient light and body tissue. In an example, a light filter can partially absorb and/or block light transmission between body tissue and a light receiver. In an example, one or more light filters can partially absorb and/or block one or more selected light wavelengths, wavelength ranges, frequencies, and/or frequency ranges. In an example, a light filter can absorb and/or block infrared or ultraviolet light. In an example, a light filter can selectively allow transmission of only infrared light or only ultraviolet light. In an example, a light filter can be made from one or more materials selected from the group consisting of: acrylic, crystal, glass, high-durometer plastic, low-durometer plastic, optical-pass material, polycarbonate, polyethylene, polymer, polyurethane, resin, sapphire, and transparent polymer. In an example, a light filter can be made by adding a light-absorbing dye to acrylic, crystal, glass, plastic, polycarbonate, polyethylene, polymer, polyurethane, resin, and/or a transparent polymer.

In an example, the wearable component of this system can include one or more lenses. In an example, the wearable component of this system can include a lens which selectively refracts and/or focuses light. In an example, a lens can selectively refract and/or focus light transmission between a light emitter and body tissue. In an example, a lens can selectively refract and/or focus light transmission between ambient light and body tissue. In an example, a lens can selectively refract and/or focus light transmission between body tissue and a light receiver. In an example, a lens can be selected from the group consisting of: biconcave, biconvex, collimating, columnar, concave, converging, convex, diverging, fluid lens, Fresnel, multiple lenses, negative meniscus, planoconcave, planoconvex, polarizing, positive meniscus, prismatic, and variable-focal lens. In an example, a lens can be made from one or more materials selected from the group consisting of: acrylic, crystal, glass, high-durometer plastic, low-durometer plastic, optical-pass material, polycarbonate, polyethylene, polymer, polyurethane, resin, sapphire, and transparent polymer.

In an example, a spectroscopic sensor of this system can include an array of light (energy) emitters. In an example, different emitters in this array can be configured to have different locations relative to the person's body. In an example, different emitters in this array can emit light at different angles with respect to the surface of a person's body. In an example, different emitters in this array can emit light at different wavelengths and/or with different light spectral distributions. In an example, different emitters in this array can emit light with different levels of coherence.

In an example, a spectroscopic sensor of this system can include a first light emitter and a second light emitter. In an example, the first light emitter can have a first location relative to the person's body and the second light emitter can have a second location relative to the person's body. In an example, the first light emitter can emit light at a first angle with respect to the surface of a person's body and the second light emitter can emit light at a second angle with respect to the surface of a person's body. In an example, the first light emitter can emit light with a first wavelength (or spectral distribution) and the second light emitter can emit light with a second wavelength (or spectral distribution). In an example, the first light emitter can emit coherent light and the second light emitter can emit non-coherent light.

In an example, a first light emitter can emit light during a first time period and a second light emitter can emit light during a second time period. In an example, the first light emitter can emit light during a first environmental condition and the second light emitter can emit light during a second environmental condition. In an example, the first light emitter can emit light when the person is engaged in a first type of physical activity and the second light emitter can emit light when the person is engaged in a second type of physical activity.

In an example, different emitters in this array emit light at different times. In an example, different emitters in this array emit light based on data from one or more biometric sensors detecting different biological or physiological parameters or conditions. In an example, different emitters in this array emit light based on data from one or more biometric sensors when a person is engaged in different types of activities. In an example, different emitters in this array emit light based on data from one or more environmental sensors in response to different environmental parameters or conditions.

In an example, different emitters in this array can emit light with different wavelengths or wavelength ranges. In an example, different emitters in this array can emit light with different wavelengths or wavelength ranges based on data from one or more biometric sensors detecting different biological or physiological parameters or conditions. In an example, different emitters in this array can emit light with different wavelengths or wavelength ranges based on data from one or more biometric sensors when a person is engaged in different types of activities. In an example, different emitters in this array can emit light with different wavelengths or wavelength ranges based on data from one or more environmental sensors in response to different environmental parameters or conditions.

In an example, different emitters in this array can emit light at different angles with respect to a body surface. In an example, different emitters in this array can emit light at different angles with respect to a body surface based on data from one or more biometric sensors detecting different biological or physiological parameters or conditions. In an example, different emitters in this array can emit light at different angles with respect to a body surface based on data from one or more biometric sensors when a person is engaged in different types of activities. In an example, different emitters in this array can emit light at different angles with respect to a body surface based on data from one or more environmental sensors in response to different environmental parameters or conditions.

In an example, a light emitter of this system can be automatically moved by an actuator relative to a wearable housing which holds it. In an example, a light emitter can be automatically tilted by an actuator. In an example, a light emitter can be automatically rotated by an actuator. In an example, a light emitter can be automatically raised or lowered by an actuator. In an example, a light emitter can be automatically tilted, rotated, raised, or lowered when the wearable housing which holds it moves relative to the body surface on which it is worn. In an example, a light emitter can be automatically tilted, rotated, raised, or lowered in order to maintain a selected distance (or distance range) from the surface of a person's body. In an example, a light emitter can be automatically tilted, rotated, raised, or lowered in order to maintain a selected angle (or angle range) with respect to the surface of a person's body.

In an example, the beam of light emitted by a light emitter can be automatically moved by using an actuator to automatically move a lens through which this beam is transmitted. In an example, the beam of light emitted by a light emitter can be automatically moved by using an actuator to automatically rotate, tilt, raise, or lower a lens through which this beam is transmitted. In an example, the beam of light emitted by a light emitter can be automatically moved by using an actuator to automatically change the focal distance of a lens through which this beam is transmitted. In an example, the beam of light emitted by a light emitter can be automatically moved by using an actuator to automatically move a light guide through which this beam is transmitted. In an example, the beam of light emitted by a light emitter can be automatically moved by using an actuator to automatically rotate, tilt, raise, or lower a light guide through which this beam is transmitted. In an example, the beam of light emitted by a light emitter can be automatically moved by using an actuator to automatically move a light reflector (such as a mirror) from which this beam is reflected. In an example, the beam of light emitted by a light emitter can be automatically moved by using an actuator to automatically rotate, tilt, raise, or lower a light reflector (such as a mirror) from which this beam is reflected.

In an example, a first light emitter can emit light energy with a first light wavelength (or wavelength range or spectral distribution) and a second light emitter can simultaneously emit light energy with a second light wavelength (or wavelength range or spectral distribution) during the same time period. In an example, a first light emitter can emit light energy with a first light wavelength (or wavelength range or spectral distribution) and a second light emitter can simultaneously emit light energy with a second light wavelength (or wavelength range or spectral distribution) during the same time period in order to measure different physiological parameters, analytes, or conditions.

In an example, a light emitter can emit light energy with a first light wavelength (or wavelength range or spectral distribution) during a first time period and can emit light energy with a second light wavelength (or wavelength range or spectral distribution) during a second time period. In an example, a light emitter can emit light energy with a first light wavelength (or wavelength range or spectral distribution) during a first time period and can emit light energy with a second light wavelength (or wavelength range or spectral distribution) during a second time period in order to measure different physiological parameters, analytes, or conditions. In an example, a light emitter can automatically cycle through light energy emissions with a variety of wavelengths (or wavelength ranges or spectral distributions) during different time periods in order to measure different physiological parameters, analytes, or conditions.

In an example, a light emitter can emit light energy with a first light wavelength (or wavelength range or spectral distribution) during a first time period and can emit light energy with a second light wavelength (or wavelength range or spectral distribution) during a second time period in response to changing environmental conditions. In an example, a light emitter can emit light energy with a first light wavelength (or wavelength range or spectral distribution) during a first time period and can emit light energy with a second light wavelength (or wavelength range or spectral distribution) during a second time period in response to changing biometric results. In an example, a light emitter can emit light energy with a first light wavelength (or wavelength range or spectral distribution) during a first time period and can emit light energy with a second light wavelength (or wavelength range or spectral distribution) during a second time period in response to changing physiological conditions.

In an example, the wearable component of this system can include one or more light (energy) receivers. A light (energy) receiver can also be referred to as a light detector, optical detector, optical sensor, or spectroscopic sensor. In an example, a light receiver can be a spectroscopic sensor which receives light energy data which is then used to analyze the spectral distribution of light received. In an example, one or more light receivers can be configured to receive light energy which has been reflected from, passed through, and/or scattered by body tissue, organs, and/or fluid.

In an example, the wearable component of this system can include one or more light receivers which are selected from the group consisting of: avalanche photodiode (APD), charge-coupled device (CCD), complementary metal-oxide semiconductor (CMOS), digital camera, field effect transistor, infrared detector, infrared photoconductor, infrared photodiode, light dependent resistor (LDR), light energy sensor, microbolometer, optical detector, optical sensor, photoconductor, photodetector, photodiode, photomultiplier, photoresistor, phototransistor, and spectroscopic sensor.

In an example, the wearable component of this system can include one or more light receivers which are in direct optical communication with body tissue, organs, and/or fluid and directly receive light energy which has been reflected from, passed through, and/or scattered by the body tissue, organs, and/or fluid. In an example, one or more light receivers can receive light energy which has been reflected from, passed through, and/or scattered by body tissue, organs, and/or fluid indirectly via one or more light guides.

In an example, the wearable component of this system can include one or more light receivers which receive light energy that has been reflected from, passed through, and/or scattered by body tissue, organs, and/or fluid. In an example, this system can collect data concerning changes in the spectral distribution, intensity, and/or polarization of light that has been reflected from, passed through, and/or scattered by body tissue, organs, and/or fluid. In an example, this system can collect data concerning changes in the spectral distribution, intensity, and/or polarization of light that has been reflected from, passed through, and/or scattered by skin, epidermis, blood, blood vessels, intercellular fluid, lymph, muscle tissue, nerve tissue, or other body tissue or fluids.

In an example, this system can collect light energy data which is used to measure changes in the chemical composition and/or physical configuration of skin, blood, blood vessels, intercellular fluid, and/or muscles based on how the spectral distribution of light is changed by being reflected from, or passing through, the skin, blood, blood vessels, intercellular fluid, and/or muscles. In an example, this system can direct, guide, focus, and/or concentrate light energy toward body tissue, organs, and/or fluid in order to measure changes in light after that light has been reflected from, or passed through, that body tissue, organs, and/or fluid.

In an example, the wearable component of this system can include one or more light receivers which receive light energy which was originally emitted by a wearable light emitter and then subsequently reflected from, passed through, or scattered by body tissue, organs, and/or fluid. In an example, a wearable light receiver can be optically isolated from a wearable light emitter by means of a light blocking layer, coating, cladding, or component so that only light reflected from, or having passed through, body tissue, organs, or fluid reaches the light receiver.

In an example, light receivers can receive light energy from an ambient light source that has been reflected from, passed through, or scattered by body tissue, organs, and/or fluid. In an example, an ambient light source can be solar radiation. In an example, an ambient light source can be outdoor artificial lighting. In an example, ambient light source can be indoor artificial lighting. In an example, a wearable light receiver can be optically isolated from a wearable light emitter by means of a light blocking layer, coating, cladding, or component so that only ambient light reflected from, or having passed through, body tissue, organs, or fluid reaches the light receiver.

In an example, the wearable component of this system can include one or more light-blocking layers, coatings, claddings, and/or components. In an example, the wearable component of this system can include one or more light-reflecting layers, coatings, claddings, and/or components. In an example, the wearable component of this system can include one or more mirrors. In an example, a light-blocking and/or light-reflecting layer, coating, and/or cladding can be opaque. In an example, a light-blocking and/or light-reflecting layer, coating, and/or cladding can comprise a black or sliver coating. In an example, a light-blocking and/or light-reflecting layer, coating, and/or cladding can be Mylar. In an example, a light-blocking and/or light-reflecting layer, coating, and/or cladding can prevent the direct transmission of light from a light emitter to a light receiver apart from reflection from, or passing through, body tissue. In an example, a light-blocking and/or light-reflecting layer, coating, and/or cladding can optically isolate a light receiver from ambient light. In an example, a light-blocking and/or light-reflecting layer, coating, and/or cladding can reduce or prevent the direct transmission of ambient light to a light receiver apart from reflection from, or passing through, body tissue. In an example, a light-blocking and/or light-reflecting layer, coating, and/or cladding can reduce or prevent the transmission of any ambient light to a light receiver.

In an example, the wearable component of this system can include an array of light (energy) receivers. In an example, different receivers in this array can be configured to have different locations relative to the person's body. In an example, different receivers in this array can receive light at different angles with respect to the surface of a person's body. In an example, different receivers in this array can receive light at different wavelengths and/or with different light spectral distributions. In an example, different receivers in this array can receive light at different times. In an example, different receivers in this array can receive light during different environmental conditions. In an example, different receivers in this array can receive light when the person is engaged in different types of physical activities.

In an example, the wearable component of this system can include a first light receiver and a second light receiver. In an example, the first light receiver can have a first location relative to the person's body and the second light receiver can have a second location relative to the person's body. In an example, the first light receiver can receive light at a first angle with respect to the surface of a person's body and the second light receiver can receive light at a second angle with respect to the surface of a person's body. In an example, the first light receiver can receive light with a first wavelength (or spectral distribution) and the second light receiver can receive light with a second wavelength (or spectral distribution). In an example, the first light receiver can receive light during a first time period and the second light receiver can receive light during a second time period. In an example, the first light receiver can receive light during a first environmental condition and the second light receiver can receive light during a second environmental condition. In an example, the first light receiver can receive light when the person is engaged in a first type of physical activity and the second light receiver can receive light when the person is engaged in a second type of physical activity.

In an example, a light emitter can emit light along a first vector and a light receiver can receive light along a second vector. In an example, the second vector can be substantially reversed from and parallel to the first vector. In an example, a beam of light can: be emitted by the light emitter along a first vector; pass through the first (transmissive) side of an angled one-way mirror; hit body tissue; reflect back from the body tissue; reflect off the second (reflective) side of the angled one-way mirror; reflect off a second mirror; and enter the light receiver along a second vector which is reversed from and parallel to the first vector.

In an example, a beam of light can: be emitted by the light emitter along a first vector; hit body tissue; reflect back from the body tissue; pass through a lens; and enter the light receiver along a second vector which is reversed from and parallel to the first vector. In an example, a beam of light can: be emitted by the light emitter along a first vector; hit body tissue; reflect back from the body tissue; pass through a rotating and/or tilting lens; and enter the light receiver along a second vector which is reversed from and parallel to the first vector. In an example, a beam of light can: be emitted by the light emitter along a first vector; hit body tissue; reflect back from the body tissue; pass through a lens which is rotated and/or tilted by an actuator; and enter the light receiver along a second vector which is reversed from and parallel to the first vector.

In an example, a beam of light can: be emitted by the light emitter along a first vector; hit body tissue; reflect back from the body tissue; pass through a light guide; and enter the light receiver along a second vector which is reversed from and parallel to the first vector. In an example, a beam of light can: be emitted by the light emitter along a first vector; hit body tissue; reflect back from the body tissue; pass through a rotating and/or tilting light guide; and enter the light receiver along a second vector which is reversed from and parallel to the first vector. In an example, a beam of light can: be emitted by the light emitter along a first vector; hit body tissue; reflect back from the body tissue; pass through a light guide which is rotated and/or tilted by an actuator; and enter the light receiver along a second vector which is reversed from and parallel to the first vector.

In an example, a light emitter can emit light along a first vector and a light receiver can receive light along a second vector. In an example, the second vector can be substantially parallel and coaxial with the first vector. In an example, a beam of light can: be emitted by the light emitter along a first vector; hit body tissue; reflect back from the body tissue; and enter the light receiver along a second vector which is parallel and coaxial with the first vector.

In an example, a light emitter can emit light along a first vector and a light receiver can receive light along a second vector. In an example, the second vector can be substantially perpendicular to the first vector. In an example, a beam of light can: be emitted by the light emitter along a first vector; pass through the first (transmissive) side of an angled one-way mirror; hit body tissue; reflect back from the body tissue; reflect off the second (reflective) side of the angled one-way mirror; and enter the light receiver along a second vector which is perpendicular to the first vector.

In an example, a light emitter can emit light along a first vector and a light receiver can receive light along a second vector. In an example, the second vector can be reversed from the first vector and symmetric to the first vector with respect to a virtual vector extending outward in a perpendicular manner from the surface of a person's body. In an example, a beam of light can: be emitted by the light emitter along a first vector; hit body tissue at an acute angle with respect to the virtual vector; reflect off the body tissue at an actuate angle with respect to the virtual vector; and enter the light receiver along a second vector. In an example, the first and second vectors can be reversed and symmetric to each other, wherein the symmetry is with respect to the virtual vector.

In an example, the wearable component of this system can comprise one or more paired sets of light emitters and light receivers. In an example, each paired set can be configured so that light emitted from the light receiver is received by the light receiver after the light is reflected from, or passes through, body tissue or fluid. In an example, different sets of light emitters and receivers can have different angles at which they reflect light from a body surface. In an example, a first set comprising a light emitter and a light receiver can reflect light from a body surface at a first angle and a second set comprising a light emitter and a light receiver can reflect light from a body surface at a second angle. In an example, an array of sets can optimally measure light reflected from a body surface at different angles. In an example, at least one of these sets can optimally measure light reflected from a body surface at an angle which is substantially perpendicular to the body surface, regardless of the angle of the wearable component relative to the body surface. In an example, an array of sets of light emitters and receivers can measure light reflected from, or having passed through, body tissue even if the wearable component on which houses the sets moves, shifts, and/or rotates relative to the body surface.

In an example, a light receiver of this system can be automatically moved relative to a wearable housing which holds it. In an example, a light receiver can be automatically tilted, rotated, raised, or lowered by an actuator. In an example, a light receiver can be automatically tilted, rotated, raised, or lowered if the wearable housing which holds it moves relative to the body surface on which it is worn. In an example, a light receiver can be automatically tilted, rotated, raised, or lowered in order to maintain a selected distance (or distance range) from the surface of a person's body. In an example, a light receiver can be automatically tilted, rotated, raised, or lowered in order to maintain a selected angle (or angle range) with respect to the surface of a person's body.

In an example, the path of light received by a light receiver can be automatically shifted by using an actuator to automatically move a lens through which this beam is transmitted. In an example, the path of light received by a light receiver can be automatically shifted by using an actuator to automatically rotate, tilt, raise, or lower a lens through which this light travels. In an example, the path of light received by a light receiver can be automatically shifted by using an actuator to automatically change the focal distance of a lens through which this light travels. In an example, the path of light received by a light receiver can be automatically shifted by using an actuator to automatically move a light guide through which this light travels. In an example, the path of light received by a light receiver can be automatically shifted by using an actuator to automatically rotate, tilt, raise, or lower a light guide through which this light travels. In an example, the path of light received by a light receiver can be automatically shifted by using an actuator to automatically move a light reflector (such as a mirror) from which this light is reflected. In an example, the path of light received by a light receiver can be automatically shifted by using an actuator to automatically rotate, tilt, raise, or lower a light reflector (such as a mirror) from which this light is reflected.

In an example, a first light receiver can receive light energy with a first light wavelength (or wavelength range or spectral distribution) and a second light receiver can simultaneously receive light energy with a second light wavelength (or wavelength range or spectral distribution) during the same time period. In an example, a first light receiver can receive light energy with a first light wavelength (or wavelength range or spectral distribution) and a second light receiver can simultaneously receive light energy with a second light wavelength (or wavelength range or spectral distribution) during the same time period in order to simultaneously measure different physiological parameters, analytes, or conditions.

In an example, a light receiver can receive light energy with a first light wavelength (or wavelength range or spectral distribution) during a first time period and can receive light energy with a second light wavelength (or wavelength range or spectral distribution) during a second time period. In an example, a light receiver can receive light energy with a first light wavelength (or wavelength range or spectral distribution) during a first time period and can receive light energy with a second light wavelength (or wavelength range or spectral distribution) during a second time period in order to measure different physiological parameters, analytes, or conditions. In an example, a light receiver can automatically cycle through light energy emissions with a variety of wavelengths (or wavelength ranges or spectral distributions) during a different time periods in order to measure different physiological parameters, analytes, or conditions.

In an example, a light receiver can receive light energy with a first light wavelength (or wavelength range or spectral distribution) during a first time period and can receive light energy with a second light wavelength (or wavelength range or spectral distribution) during a second time period in response to changing environmental conditions. In an example, a light receiver can receive light energy with a first light wavelength (or wavelength range or spectral distribution) during a first time period and can receive light energy with a second light wavelength (or wavelength range or spectral distribution) during a second time period in response to changing biometric results. In an example, a light receiver can receive light energy with a first light wavelength (or wavelength range or spectral distribution) during a first time period and can receive light energy with a second light wavelength (or wavelength range or spectral distribution) during a second time period in response to changing physiological conditions.

In an example, a spectroscopic sensor of this system can be configured to receive light energy which has been reflected from, or passed through, body tissue, organs, and/or fluid selected from the group consisting of: aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, saliva, skin, sweat, and tears.

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, saliva, skin, sweat, and/or tears in order to monitor oxygen levels (or changes in those levels). In an example, a spectroscopic sensor of this system can be configured to receive light energy which has been reflected from, or passed through, blood in order to monitor blood oxygen levels (or changes in those levels). In an example, the operation of the implanted cardiac management device can be adjusted based on detected tissue and/or blood oxygen levels (or changes in those levels). In this manner, the person's cardiac functioning can be adjusted based on detected tissue and/or blood oxygen levels (or changes in those levels).

In an example, this system can increase the frequency of a heart beats via an implanted cardiac management device based on low oxygen levels detected in body tissue and/or fluid via a wearable spectroscopic sensor. In an example, this system can increase the magnitude of heart contractions via an implanted cardiac management device based on low oxygen levels detected in body tissue and/or fluid via a wearable spectroscopic sensor. In an example, this system can increase the frequency, regularity, magnitude, and/or coordination of heart muscle contractions via an implanted cardiac management device based on low oxygen levels detected using a wearable spectroscopic sensor. In an example, this system can increase the frequency, regularity, magnitude, and/or coordination of a person's heart muscle contractions via an implanted cardiac management device based on low oxygen levels detected using a wearable spectroscopic sensor.

More generally, this system can increase the frequency of a heart beats via an implanted cardiac management device based on low oxygen levels detected in body tissue and/or fluid via a wearable biometric sensor. In an example, this system can increase the magnitude of heart contractions via an implanted cardiac management device based on low oxygen levels detected in body tissue and/or fluid via a wearable biometric sensor. In an example, this system can increase the frequency, regularity, magnitude, and/or coordination of heart muscle contractions via an implanted cardiac management device based on low oxygen levels detected using a wearable light energy sensor or electromagnetic energy sensor. In an example, this system can increase the frequency, regularity, magnitude, and/or coordination of a person's heart muscle contractions via an implanted cardiac management device based on low oxygen levels detected using a wearable spectroscopic sensor or EEG sensor.

In an example, this system can adjust parameters of cardiac functioning in response to low oxygen levels which are detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, this system can comprise a (partially or fully) closed-loop system for automatic adjustment of cardiac functioning via an implanted cardiac management device based on data from one or more wearable biometric sensors. These sensors can include one or more wearable spectroscopic sensors. In an example, automatic adjustment of cardiac functioning in response to detection of an abnormal biometric parameter value can help to restore underlying biological and/or physiological processes to their proper functioning. For example, detection of low oxygen levels in peripheral tissue (or organs) by a wearable biometric sensor can trigger increased blood flow, which in turn can help to restore proper oxygen levels for that tissue (or organs). The ability to measure oxygen levels via sensors at one or more peripheral locations can provide more accurate measures of body-wide oxygenation than, for example, a single central sensor.

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, saliva, skin, sweat, and/or tears in order to monitor lactate (and/or lactic acid) levels (or changes in those levels). In an example, the operation of the implanted cardiac management device can be adjusted based on detected lactate (and/or lactic acid) levels (or changes in those levels). In this manner, the person's cardiac functioning can be adjusted based on detected lactate (and/or lactic acid) levels (or changes in those levels).

In an example, this system can increase the frequency of a person's heart beats via an implanted cardiac management device based on high lactate levels detected in the person's body tissue and/or fluid via a wearable spectroscopic sensor. In an example, this system can increase the magnitude of a person's heart contractions via an implanted cardiac management device based on high lactate levels detected using a wearable spectroscopic sensor. In an example, this system can increase the frequency, regularity, magnitude, and/or coordination of a person's heart muscle contractions via an implanted cardiac management device based on high lactate levels detected in the person's body tissue and/or fluid via a wearable spectroscopic sensor. In an example, this system can increase the frequency, regularity, magnitude, and/or coordination of a person's heart muscle contractions via an implanted cardiac management device based on high lactate levels detected using a wearable spectroscopic sensor.

More generally, this system can increase the frequency of a person's heart beats via an implanted cardiac management device based on high lactate levels detected in the person's body tissue and/or fluid via a wearable biometric sensor. In an example, this system can increase the magnitude of a person's heart contractions via an implanted cardiac management device based on high lactate levels detected using a wearable biometric sensor. In an example, this system can increase the frequency, regularity, magnitude, and/or coordination of a person's heart muscle contractions via an implanted cardiac management device based on high lactate levels detected in the person's body tissue and/or fluid via a wearable light energy or electromagnetic energy sensor. In an example, this system can increase the frequency, regularity, magnitude, and/or coordination of a person's heart muscle contractions via an implanted cardiac management device based on high lactate levels detected using a wearable spectroscopic sensor or EEG sensor.

In an example, this system can adjust parameters of cardiac functioning in response to high lactate levels which are detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor carbon dioxide levels and/or changes in carbon dioxide levels. In an example, the operation of the implanted cardiac management device can be adjusted based on detected carbon dioxide levels and/or changes in carbon dioxide levels. In this manner, the person's cardiac functioning can be adjusted based on detected carbon dioxide levels and/or changes in carbon dioxide levels.

In an example, this system can increase the frequency of a person's heart beats via an implanted cardiac management device based on high carbon dioxide levels detected in the person's body tissue and/or fluid via a wearable spectroscopic sensor. In an example, this system can increase the magnitude of a person's heart contractions via an implanted cardiac management device based on high carbon dioxide levels detected in the person's body tissue and/or fluid via a wearable spectroscopic sensor. In an example, this system can increase the frequency, regularity, magnitude, and/or coordination of a person's heart muscle contractions via an implanted cardiac management device based on high carbon dioxide levels detected in the person's body tissue and/or fluid via a wearable spectroscopic sensor. In an example, this system can increase the frequency, regularity, magnitude, and/or coordination of a person's heart muscle contractions via an implanted cardiac management device based on high carbon dioxide levels detected in the person's body tissue and/or fluid via a wearable spectroscopic sensor.

More generally, this system can increase the frequency of a person's heart beats via an implanted cardiac management device based on high carbon dioxide levels detected in the person's body tissue and/or fluid via a wearable biometric sensor. In an example, this system can increase the magnitude of a person's heart contractions via an implanted cardiac management device based on high carbon dioxide levels detected in the person's body tissue and/or fluid via a wearable biometric sensor. In an example, this system can increase the frequency, regularity, magnitude, and/or coordination of a person's heart muscle contractions via an implanted cardiac management device based on high carbon dioxide levels detected in the person's body tissue and/or fluid via a wearable light energy and/or electromagnetic energy sensor. In an example, this system can increase the frequency, regularity, magnitude, and/or coordination of a person's heart muscle contractions via an implanted cardiac management device based on high carbon dioxide levels detected in the person's body tissue and/or fluid via a wearable spectroscopic sensor, EMG sensor, or EEG sensor.

In an example, this system can adjust parameters of cardiac functioning in response to high carbon dioxide levels which are detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor glucose levels and/or changes in glucose levels. In an example, the operation of the implanted cardiac management device can be adjusted based on detected glucose levels and/or changes in glucose levels. In this manner, the person's cardiac functioning can be adjusted based on detected glucose levels and/or changes in glucose levels.

In an example, this system can increase (or decrease) the frequency of a person's heart beats via an implanted cardiac management device based on low (or high) glucose levels detected in the person's body tissue and/or fluid via a wearable biometric sensor. In an example, this system can increase (or decrease) the magnitude of a person's heart contractions via an implanted cardiac management device based on low (or high) glucose levels detected in the person's body tissue and/or fluid via a wearable biometric sensor. In an example, this system can increase (or decrease) the frequency, regularity, magnitude, and/or coordination of a person's heart muscle contractions via an implanted cardiac management device based on low (or high) glucose levels detected in the person's body tissue and/or fluid via a wearable light energy and/or electromagnetic energy sensor. In an example, this system can increase (or decrease) the frequency, regularity, magnitude, and/or coordination of a person's heart muscle contractions via an implanted cardiac management device based on low (or high) glucose levels detected in the person's body tissue and/or fluid via a wearable spectroscopic sensor, tissue impedance sensor, or EEG sensor.

In an example, this system can adjust parameters of cardiac functioning in response to abnormal glucose levels which are detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor for troponin and/or changes in troponin. In an example, the operation of the implanted cardiac management device can be adjusted based on detected troponin and/or changes in troponin. In this manner, the person's cardiac functioning can be adjusted based on detected troponin level and/or changes in troponin level.

In an example, this system can adjust parameters of cardiac functioning in response to troponin which is detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor electrolyte levels and/or changes in electrolyte levels. In an example, the operation of the implanted cardiac management device can be adjusted based on detected electrolyte levels and/or changes in electrolyte levels. In this manner, the person's cardiac functioning can be adjusted based on detected electrolyte levels and/or changes in electrolyte levels. In an example, a spectroscopic sensor of this system can detect the compositions of blood, sweat, and tears. For example, “Spinning Wheel” was a great one.

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor water levels and/or changes in hydration and/or water level. In an example, the operation of the implanted cardiac management device can be adjusted based on detected hydration and/or water level and/or changes therein. In this manner, the person's cardiac functioning can be adjusted based on hydration and/or water level or changes therein.

In an example, this system can adjust parameters of cardiac functioning in response to abnormal water levels which are detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor body temperature and/or changes in body temperature. In an example, the operation of the implanted cardiac management device can be adjusted based on detected body temperature and/or changes in body temperature. In this manner, the person's cardiac functioning can be adjusted based on detected body temperature and/or changes in body temperature.

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor pH levels and/or changes in pH levels. In an example, the operation of the implanted cardiac management device can be adjusted based on detected pH level and/or changes in pH level. In this manner, the person's cardiac functioning can be adjusted based on detected body pH level and/or changes in body pH level.

In an example, a spectroscopic sensor of this system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor hormone levels and/or changes in hormone levels. In an example, the operation of the implanted cardiac management device can be adjusted based on detected hormone levels and/or changes in hormone levels. In this manner, the person's cardiac functioning can be adjusted based on detected hormone levels and/or changes in hormone levels.

In an example, a spectroscopic sensor of this system can be configured to receive light energy which has been reflected from, or passed through, a person's blood and/or blood vessels in order to monitor blood pressure and/or changes in blood pressure. In an example, the operation of the implanted cardiac management device can be adjusted based on detected blood pressure and/or changes in blood pressure. In this manner, the person's cardiac functioning can be adjusted based on detected blood pressure and/or changes in blood pressure.

In an example, this system can increase (or decrease) the frequency of a person's heart beats via an implanted cardiac management device based on low (or high) blood pressure detected via a wearable spectroscopic sensor. In an example, this system can increase (or decrease) the magnitude of a person's heart contractions via an implanted cardiac management device based on a low (or high) blood pressure level in the person's body tissue and/or fluid via a wearable spectroscopic sensor. In an example, this system can increase (or decrease) the frequency, regularity, magnitude, and/or coordination of a person's heart muscle contractions via an implanted cardiac management device based on a low (or high) blood pressure level detected in the person's body tissue and/or fluid via a wearable spectroscopic sensor. In an example, this system can increase (or decrease) the frequency, regularity, magnitude, and/or coordination of a person's heart muscle contractions via an implanted cardiac management device based on a low (or high) blood pressure level detected in the person's body tissue and/or fluid via a wearable spectroscopic sensor.

More generally, this system can increase (or decrease) the frequency of a person's heart beats via an implanted cardiac management device based on low (or high) blood pressure detected via a wearable biometric sensor. In an example, this system can increase (or decrease) the magnitude of a person's heart contractions via an implanted cardiac management device based on a low (or high) blood pressure level in the person's body tissue and/or fluid via a wearable biometric sensor. In an example, this system can increase (or decrease) the frequency, regularity, magnitude, and/or coordination of a person's heart muscle contractions via an implanted cardiac management device based on a low (or high) blood pressure level detected in the person's body tissue and/or fluid via a wearable light energy and/or electromagnetic energy sensor. In an example, this system can increase (or decrease) the frequency, regularity, magnitude, and/or coordination of a person's heart muscle contractions via an implanted cardiac management device based on a low (or high) blood pressure level detected in the person's body tissue and/or fluid via a wearable spectroscopic sensor or EEG sensor.

In an example, this system can adjust parameters of cardiac functioning in response to an abnormal blood pressure level which is detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, this system can comprise a (partially or fully) closed-loop system for automatic adjustment of cardiac functioning via an implanted cardiac management device based on data from one or more wearable biometric sensors. These sensors can include one or more wearable spectroscopic sensors. In an example, automatic adjustment of cardiac functioning in response to detection of an abnormal biometric parameter value can help to restore underlying biological and/or physiological processes to their proper functioning. For example, detection of low blood pressure in peripheral tissue (or organs) by a wearable biometric sensor can trigger increased blood flow, which in turn can help to restore proper blood pressure in that tissue (or organs). For example, detection of high blood pressure in peripheral tissue (or organs) by a wearable biometric sensor can trigger decreased blood flow, which in turn can help to restore proper blood pressure in that tissue (or organs). The ability to measure blood pressure values via sensors at one or more peripheral locations can provide more accurate measures of body-wide cardiovascular dynamics than, for example, a single central sensor.

In an example, a spectroscopic sensor of this system can be configured to receive light energy which has been reflected from, or passed through, a person's blood and/or blood vessels in order to monitor heart rate and/or changes in heart rate. In an example, a spectroscopic sensor of this system can be configured to receive light energy which has been reflected from, or passed through, a person's blood and/or blood vessels in order to monitor for tachycardia or bradycardia. In an example, a spectroscopic sensor of this system can be configured to receive light energy which has been reflected from, or passed through, a person's blood and/or blood vessels in order to monitor for heart rate variability (HRV) and/or an irregular heartbeat. In an example, the operation of the implanted cardiac management device can be adjusted based on detected heart rate and/or changes in heart rate. In an example, the operation of the implanted cardiac management device can be adjusted based on detection of tachycardia, bradycardia, and/or an irregular heartbeat. In this manner, the person's cardiac functioning can be adjusted based on peripherally-detected heart rate and/or changes in peripherally-detected heart rate.

In an example, this system can increase the frequency of a person's heart beats via an implanted cardiac management device based on low heart rate detected via a wearable spectroscopic sensor. In an example, this system can increase the magnitude of a person's heart contractions via an implanted cardiac management device based on a low heart rate level in the person's body tissue and/or fluid via a wearable spectroscopic sensor. In an example, this system can increase the frequency, regularity, magnitude, and/or coordination of a person's heart muscle contractions via an implanted cardiac management device based on a low heart rate level detected in the person's body tissue and/or fluid via a wearable spectroscopic sensor. In an example, this system can increase the frequency, regularity, magnitude, and/or coordination of a person's heart muscle contractions via an implanted cardiac management device based on a low (or high) heart rate level detected in the person's body tissue and/or fluid via a wearable spectroscopic sensor.

More generally, this system can increase the frequency of a person's heart beats via an implanted cardiac management device based on low heart rate detected via a wearable biometric sensor. In an example, this system can increase the magnitude of a person's heart contractions via an implanted cardiac management device based on a low heart rate level in the person's body tissue and/or fluid via a wearable biometric sensor. In an example, this system can increase the frequency, regularity, magnitude, and/or coordination of a person's heart muscle contractions via an implanted cardiac management device based on a low heart rate level detected in the person's body tissue and/or fluid via a wearable light energy and/or electromagnetic energy sensor. In an example, this system can increase the frequency, regularity, magnitude, and/or coordination of a person's heart muscle contractions via an implanted cardiac management device based on a low (or high) heart rate level detected in the person's body tissue and/or fluid via a wearable spectroscopic sensor or EEG sensor.

In an example, this system can adjust parameters of cardiac functioning in response to an abnormal peripherally-detected heart rate which is detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a spectroscopic sensor of this system can be configured to collect data concerning light energy reflected from, or having passed through, blood and/or blood vessels in order to measure one or more biometric parameters or conditions selected from the group consisting of: albumin level, bilirubin level, blood flow, blood glucose level, blood hydration, blood oxygen (SpO2), blood pH level, blood pressure, blood pulsation, blood urea nitrogen (BUN), blood vessel dilation, blood volume, body hydration, calcium level, caloric intake, caloric metabolism, carbon dioxide level, carbon dioxide level, carbon monoxide level, carboxyhemoglobin level, chloride level, cholesterol (HDL) level, cholesterol (LDL) level, copper level, creatine kinase level, creatine level, creatine phosphokinase level, electrolyte levels, glucose level, heart rate, heart rate variability (HRV), hemoglobin level, hormone level, hydration, hypertension, iron level, lactate level, lactic acid level, lipid levels, magnesium level, methemoglobin level, myoglobin level, nickel level, nitrogen level, oxygen level, oxygen saturation, partial pressure of carbon dioxide, partial pressure of oxygen, pH level, phosphorus level, potassium level, protein levels, pulse, sodium level, thyroid stimulating hormone (TSH) level, triglyceride level, troponin level, and urea nitrogen level.

In an example, a biometric light energy sensor of this system (such as a spectroscopic sensor) can be configured to collect data concerning one or more biometric parameters or conditions selected from the group consisting of: albumin level, anaerobic threshold, atrial fibrillation, bilirubin level, blood carbon dioxide level, blood flow, blood glucose level, blood hydration, blood oxygen (SpO2), blood pH level, blood pressure, blood pulsation, blood urea nitrogen (BUN), blood vessel dilation, blood volume, body acceleration, body balance, body configuration, body fat density, body hydration, body motion, whole-body posture, body speed, bradycardia, brainwave activity, brainwave frequency band levels, breathing rate, calcium level, caloric intake, caloric metabolism, carbon dioxide level, carbon monoxide level, carboxyhemoglobin level, cardiac output, cardiopulmonary function, chemical composition of blood, chemical composition of intercellular fluid, chemical composition of sweat, chemical composition of tears, chloride level, cholesterol (HDL) level, cholesterol (LDL) level, copper level, creatine level, creatine phosphokinase level, digestive system functioning, eating behavior, electrocardiographic (ECG) patterns, electroencephalographic (EEG) patterns, electrolyte levels, electromagnetic brain activity, electromagnetic energy from the body, electromagnetic evoked potentials of the brain, and electromyographic (EMG) patterns. In an example, this system can adjust parameters of cardiac functioning in response to abnormal values of one or more of these biometric parameters or conditions as detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a biometric light energy sensor of this system (such as a spectroscopic sensor) can be configured to collect data concerning one or more biometric parameters or conditions selected from the group consisting of: exercise level, exhaled breath volume, eye movement, galvanometric response, glucose level, GSR data, heart arrhythmia, heart rate, heart rate variability (HRV), heartbeat irregularity, hemoglobin level, hormone level, hydration, hypertension, inhaled breath volume, interstitial glucose level, intracranial pressure, iron level, jaw motion, joint angle, lactate level, lactic acid, limb acceleration, limb configuration, lipid levels, magnesium level, maximum volume of oxygen consumption, metabolism, methemoglobin level, muscle tension, myoglobin level, neurological activity level, nickel level, nitrogen level, overall body activity level, oxygen level, oxygen saturation, partial pressure of carbon dioxide, partial pressure of oxygen, perspiration level or rate, pH level, pheromone level, phosphorus level, potassium level, pressure level, protein levels, pulse, QRS, respiration rate, respiration volume, resting heart rate, skin humidity, skin impedance, skin resistance, sodium level, sound level, SpCO2, swallowing rate, sweating rate or level, tachycardia, tearing, temperature (core body), temperature (skin), thyroid stimulating hormone (TSH) level, tissue impedance, tissue oxygen level, triglyceride level, troponin level, urea nitrogen level, VO2 max, and body water level. In an example, this system can adjust parameters of cardiac functioning in response to abnormal values of one or more of these biometric parameters or conditions as detected by a wearable biometric sensor (such as a wearable spectroscopic sensor). In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a biometric sensor of this system can be an electromagnetic energy sensor. In an example, an electromagnetic energy sensor can be an electromagnetic energy receiver which receives electromagnetic energy which is naturally generated by the electromagnetic activity of body tissue and/or organs. In an example, an electromagnetic energy sensor can comprise an electromagnetic energy emitter at a first location relative to body tissue and an electromagnetic energy receiver at a second location relative to body tissue, wherein the electromagnetic energy receiver receives energy which has been transmitted from the electromagnetic energy emitter through body tissue. In an example, the electromagnetic energy receiver can collect data concerning (changes in) the conductivity, resistance, and/or impedance of electromagnetic energy transmitted through body tissue from the electromagnetic energy emitter to the electromagnetic energy receiver. In an example, an electromagnetic energy emitter and an electromagnetic energy receiver can together be referred to as an electromagnetic energy sensor.

In an example, one or more electromagnetic energy sensors can be selected from the group consisting of: action potential sensor, bipolar electrode, capacitive electrode, capacitive sensor, conductance electrode, conductance sensor, dry electrode, wet electrode, electrical resistance sensor, electrocardiographic (ECG) sensor, electrode, electroencephalographic (EEG) sensor, electromagnetic brain activity sensor, electromagnetic path, electromagnetic sensor, electromyographic (EMG) sensor, galvanic skin response (GSK) sensor, impedance sensor, inductance sensor, interferometer, magnometer, neural action potential sensor, neural impulse sensor, and piezoelectric sensor. In an example, one or more electromagnetic energy sensors can be selected from the group consisting of: electroencephalograph (EEG) sensor, electromyographic (EMG) sensor, electrocardiographic (ECG) sensor, skin and/or tissue impedance sensor, and skin and/or tissue resistance sensor.

In an example, an electromagnetic energy sensor can be an electromagnetic brain activity sensor. In an example, an electromagnetic energy sensor can be an electroencephalographic (EEG) sensor. In an example, an electromagnetic energy sensor can be a wearable electromagnetic brain activity sensor and/or wearable electroencephalographic (EEG) sensor. In an example, an electromagnetic energy sensor can be a brain activity sensor which collects data concerning the natural emission of electromagnetic energy by a person's brain. In an example, an electromagnetic energy sensor can comprise an electromagnetic energy emitter and an electromagnetic energy receiver which are in proximity to a person's head. In an example, an electromagnetic energy sensor can collect data concerning changes in transmission of electromagnetic energy from the emitter to the receiver due to changes in electromagnetic brain activity. In an example, an electromagnetic brain activity sensor can measure voltage fluctuations resulting from ionic current within the neurons of the brain.

In an example, an electromagnetic energy sensor that collects data concerning brain activity can be a capacitive sensor. In an example, an electromagnetic energy sensor that collects data concerning brain activity can be a dry electrode. In an example, an electromagnetic energy sensor which collects data concerning brain activity can be a wet electrode. In an example, an electromagnetic energy sensor which collects data concerning brain activity can measure voltage fluctuations between a first electrode and a second (reference) electrode due to electromagnetic brain activity. In an example, voltage differences between a first electrode and a second (reference) electrode can be called a “channel ” In an example, a set of channels can be called a “montage.” In an example, a second (reference) electrode can be attached to an ear. In an example, there can be two reference electrodes in a system, one attached to each ear.

In an example, the wearable component of this system can include one or more electromagnetic energy sensors which collect data concerning electromagnetic brain activity. In an example, the operation of an implanted cardiac management device can be adjusted based on detection of a selected pattern of electromagnetic brain activity from a particular electromagnetic energy sensor location, a particular channel, and/or particular montage of channels. In an example, a pattern of electromagnetic brain activity can be a change in activity in a specific area of a person's brain as measured from one or more specific sensor locations on the person's head. In an example, this pattern can be a transient pattern which is recorded from one or more locations. In an example, this pattern can be the start of a repeating pattern which is recorded from one or more locations. In an example, this pattern can be a change in an ongoing repeating pattern which is recorded from one or more locations. In an example, this pattern can be a change in electromagnetic brain activity measured from one location or channel relative to electromagnetic brain activity measured from one or more different locations or channels. In an example, one or more electromagnetic energy sensor which collects data concerning brain activities or channels can be placed at one or more electrode placement sites selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, the wearable component of this system can include an electromagnetic energy sensor which measures electromagnetic brain activity. In an example, the operation of an implanted cardiac management device can be adjusted based on detection of a selected pattern of electromagnetic brain activity from data collected by the wearable component of this system. In an example, this pattern of electromagnetic brain activity can be a repeating waveform (or pattern) of electromagnetic brain activity. In an example, a repeating electromagnetic brain activity pattern can be an oscillatory pattern.

In an example, a repeating electromagnetic brain activity pattern can be modeled as a composite of multiple sine waves. In an example, a repeating electromagnetic brain activity pattern can be decomposed into sub-patterns in different frequency bands. In an example, these frequency bands can be selected from the group consisting of: Delta, Theta, Alpha, Beta, and Gamma. Ongoing brain waveforms classified as Delta waves can be within a frequency band selected from the group consisting of: 0.5-3.5 Hz, 0.5-4 Hz, 1-3 Hz, 1-4 Hz, and 2-4 Hz. Ongoing brain waveforms classified as Theta waves can be within a frequency band selected from the group consisting of: from the group consisting of: 3.5-7 Hz, 3-7 Hz, 4-7 Hz, 4-7.5 Hz, 4-8 Hz, and 5-7 Hz. Ongoing brain waveforms classified as Alpha waves can be within a frequency band selected from the group consisting of: 7-13 Hz, 7-14 Hz, 8-12 Hz, 8-13 Hz, 7-11 Hz, 8-10 Hz, and 8-10 Hz. Ongoing brain waveforms classified as Beta waves can be within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. Ongoing brain waveforms classified as Gamma waves can be within a frequency band selected from the group consisting of: group consisting of: 30-100 Hz, 35-100 Hz, 40-100 Hz, and greater than 30 Hz.

In an example, a repeating pattern of electromagnetic brain activity may be triggered by an abnormal value for a biological parameter or condition. In an example, the human brain can function as a biological “organic sensor” for monitoring biological and/or physiological processes. The results from this “organic sensor” can be collected by one or more wearable electromagnetic energy sensors and used to adjust the implanted cardiac management device. For example, if the brain detects low tissue oxygen levels, then this changes electromagnetic brain activity patterns, which is then detected by a wearable electromagnetic energy sensor, which then adjusts the operation of the implanted cardiac management device, which then increases blood flow, which can then restore proper tissue oxygen levels.

In an example, the operation of an implanted cardiac management device can be adjusted based on detection of a transient (non-repeating) pattern of electromagnetic brain activity from data collected by the wearable component of this system. A transient pattern of electromagnetic brain activity can be a sequence of spikes or waves which do not repeat in a regular or ongoing manner. In an example, one or more parameters used to identify a transient pattern of electromagnetic brain activity can be selected from the group consisting of: shape of one or more spikes; amplitude, maximum, or minimum of one or more spikes; frequency of multiple spikes; pattern covariation; pattern entropy; pattern signature; first and second order differentials; polynomial modeling; and composite sine wave modeling.

In an example, a transient pattern of electromagnetic brain activity can be triggered by an external sensory stimulus and/or environmental event. In an example, a transient pattern of electromagnetic brain activity can be triggered by an internal biological and/or physiological event. In an example, a transient pattern of electromagnetic brain activity may be triggered by an abnormal value for a biological parameter or condition. In an example, the human brain can function as a biological “organic sensor” for monitoring biological and/or physiological processes. The results from this “organic sensor” can be collected by one or more wearable electromagnetic energy sensors and used to adjust the implanted cardiac management device. For example, if the brain detects low tissue oxygen levels, then this changes electromagnetic brain activity patterns, which is then detected by a wearable electromagnetic energy sensor, which then adjusts the operation of the implanted cardiac management device, which then increases blood flow, which can then restore proper tissue oxygen levels.

In an example, a pattern of electromagnetic brain activity which is selected to trigger adjustment of cardiac function can be identified using one or more analytical methods which are selected from the group consisting of: Analysis of Variance (ANOVA), Artificial Neural Network (ANN), Auto-Regressive (AR) Modeling, Bayesian Analysis, Bonferroni Analysis (BA), Centroid Analysis, Chi-Squared Analysis, Cluster Analysis, Correlation, Covariance, Data Normalization (DN), Decision Tree Analysis (DTA), Discrete Fourier transform (DFT), Discriminant Analysis (DA), Edgar AI Analysis, Empirical Mode Decomposition (EMD), Factor Analysis (FA), Fast Fourier Transform (FFT), Feature Vector Analysis (FVA), Fisher Linear Discriminant, Fourier Transformation (FT) Method, Fuzzy Logic (FL) Modeling, Gaussian Model (GM), Generalized Auto-Regressive Conditional Heteroscedasticity (GARCH) Modeling, Hidden Markov Model (HMM), Independent Components Analysis (ICA), Inter-Band Power Ratio, Inter-Channel Power Ratio, Inter-Montage Power Mean, Inter-Montage Ratio, Kalman Filter (KF), Kernel Estimation, Laplacian Filter, Laplacian Montage Analysis, Least Squares Estimation, Linear Regression, Linear Transform, Logit Model, Machine Learning (ML), Markov Model, Maximum Entropy Modeling, Maximum Likelihood, Mean Power, Multi-Band Covariance Analysis, Multi-Channel Covariance Analysis, Multivariate Linear Regression, Multivariate Logit, Multivariate Regression, Naive Bayes Classifier, Neural Network, Non-Linear Programming, Non-negative Matrix Factorization (NMF), Power Spectral Density, Power Spectrum Analysis, Principal Components Analysis (PCA), Probit Model, Quadratic Minimum Distance Classifier, Random Forest (RF), Random Forest Analysis (RFA), Regression Model, Signal Amplitude (SA), Signal Averaging, Signal Decomposition, Sine Wave Compositing, Singular Value Decomposition (SVD), Spine Function, Support Vector and/or Machine (SVM), Time Domain Analysis, Time Frequency Analysis, Time Series Model, Trained Bayes Classifier, Variance, Waveform Identification, Wavelet Analysis, and Wavelet Transformation.

In an example, an electromagnetic brain activity sensor can be configured to collect data concerning electromagnetic brain activity which is affected by body tissue and/or fluid oxygen levels (or changes in those levels). In an example, abnormal body tissue and/or fluid oxygen levels (or changes in those levels) can trigger changes in repeating patterns and/or transient patterns of electromagnetic brain activity. These changed patterns are detected by analysis of data from one or more wearable electromagnetic energy sensors, which triggers adjustment of cardiac function (via the implanted cardiac management device) which, in turn, restores normal body tissue and/or fluid oxygen levels.

In an example, this system can increase the frequency of a heart beats via an implanted cardiac management device when low oxygen levels are detected in body tissue and/or fluid via data from a wearable electromagnetic brain activity sensor. In an example, this system can increase the magnitude of heart contractions via an implanted cardiac management device when low oxygen levels are detected in body tissue and/or fluid via data from a wearable electromagnetic brain activity sensor. In an example, this system can increase the frequency, regularity, magnitude, and/or coordination of heart muscle contractions via an implanted cardiac management device when low oxygen levels are detected via data from a wearable electromagnetic brain activity sensor.

In an example, this system can adjust parameters of cardiac functioning in response to low oxygen levels detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, this system can comprise a (partially or fully) closed-loop system for automatic adjustment of cardiac functioning via an implanted cardiac management device based on data from an electromagnetic brain activity sensor. In an example, automatic adjustment of cardiac functioning in response to detection of abnormal biometric parameter values can help to restore underlying biological and/or physiological processes to their proper functioning. For example, detection of a low oxygen levels in the brain by a wearable electromagnetic energy sensor can trigger increased blood flow which, in turn, can help to restore proper oxygen levels for the brain. The ability to measure oxygen levels in the brain (relatively directly) can provide a more accurate and timely measure of brain oxygenation than a limb-worn sensor or centrally-implanted sensor.

In an example, an electromagnetic brain activity sensor can be configured to collect data concerning electromagnetic brain activity which is affected by body tissue and/or fluid lactate (and/or lactic acid) levels (or changes in those levels). In an example, abnormal body tissue and/or fluid lactate (and/or lactic acid) levels (or changes in those levels) can trigger changes in repeating patterns and/or transient patterns of electromagnetic brain activity. These changed patterns are detected by analysis of data from one or more wearable electromagnetic energy sensors, which triggers adjustment of cardiac function (via the implanted cardiac management device) which, in turn, lowers body tissue and/or fluid lactate (and/or lactic acid) levels.

In an example, this system can increase the frequency of a heart beats via an implanted cardiac management device when high lactate (and/or lactic acid) levels are detected in body tissue and/or fluid via data from a wearable electromagnetic brain activity sensor. In an example, this system can increase the magnitude of heart contractions via an implanted cardiac management device when high lactate (and/or lactic acid) levels are detected in body tissue and/or fluid via data from a wearable electromagnetic brain activity sensor. In an example, this system can increase the frequency, regularity, magnitude, and/or coordination of heart muscle contractions via an implanted cardiac management device when high lactate (and/or lactic acid) levels are detected via data from a wearable electromagnetic brain activity sensor.

In an example, this system can adjust parameters of cardiac functioning in response to high lactate (and/or lactic acid) levels detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, this system can comprise a (partially or fully) closed-loop system for automatic adjustment of cardiac functioning via an implanted cardiac management device based on data from an electromagnetic brain activity sensor. In an example, automatic adjustment of cardiac functioning in response to detection of abnormal biometric parameter values can help to restore underlying biological and/or physiological processes to their proper functioning. For example, detection of high lactate (and/or lactic acid) levels by a wearable electromagnetic energy sensor can trigger increased blood flow which, in turn, can help to lower lactate (and/or lactic acid) levels.

In an example, an electromagnetic brain activity sensor can be configured to collect data concerning electromagnetic brain activity which is affected by body tissue and/or fluid carbon dioxide levels (or changes in those levels). In an example, abnormal body tissue and/or fluid carbon dioxide levels (or changes in those levels) can trigger changes in repeating patterns and/or transient patterns of electromagnetic brain activity. These changed patterns are detected by analysis of data from one or more wearable electromagnetic energy sensors, which triggers adjustment of cardiac function (via the implanted cardiac management device) which, in turn, lowers body tissue and/or fluid carbon dioxide levels.

In an example, this system can increase the frequency of a heart beats via an implanted cardiac management device when high carbon dioxide levels are detected in body tissue and/or fluid via data from a wearable electromagnetic brain activity sensor. In an example, this system can increase the magnitude of heart contractions via an implanted cardiac management device when high carbon dioxide levels are detected in body tissue and/or fluid via data from a wearable electromagnetic brain activity sensor. In an example, this system can increase the frequency, regularity, magnitude, and/or coordination of heart muscle contractions via an implanted cardiac management device when high carbon dioxide levels are detected via data from a wearable electromagnetic brain activity sensor.

In an example, this system can adjust parameters of cardiac functioning in response to high carbon dioxide levels detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, this system can comprise a (partially or fully) closed-loop system for automatic adjustment of cardiac functioning via an implanted cardiac management device based on data from an electromagnetic brain activity sensor. In an example, automatic adjustment of cardiac functioning in response to detection of abnormal biometric parameter values can help to restore underlying biological and/or physiological processes to their proper functioning. For example, detection of a high carbon dioxide levels by a wearable electromagnetic energy sensor can trigger increased blood flow which, in turn, can help to lower carbon dioxide levels.

In an example, an electromagnetic brain activity sensor can be configured to collect data concerning electromagnetic brain activity which is affected by body tissue and/or fluid glucose levels (or changes in those levels). In an example, abnormal body tissue and/or fluid glucose levels (or changes in those levels) can trigger changes in repeating patterns and/or transient patterns of electromagnetic brain activity. In an example, this system can increase the frequency, regularity, magnitude, and/or coordination of heart muscle contractions via an implanted cardiac management device when abnormal glucose levels are detected via data from a wearable electromagnetic brain activity sensor. Even if changes in cardiac function do not change glucose levels, such changes may help the body better cope with abnormal glucose levels without long-term adverse effects.

In an example, this system can adjust parameters of cardiac functioning in response to abnormal glucose levels detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic brain activity sensor can be configured to collect data concerning electromagnetic brain activity which is affected by blood pressure levels (or changes in those levels). In an example, abnormal blood pressure levels (or changes in those levels) can trigger changes in repeating patterns and/or transient patterns of electromagnetic brain activity. These changed patterns are detected by analysis of data from one or more wearable electromagnetic energy sensors, which triggers adjustment of cardiac function (via the implanted cardiac management device) which, in turn, restores normal blood pressure levels.

In an example, this system can decrease the frequency of a heart beats via an implanted cardiac management device when high blood pressure levels are detected in body tissue and/or fluid via data from a wearable electromagnetic brain activity sensor. In an example, this system can decrease the magnitude of heart contractions via an implanted cardiac management device when high blood pressure levels are detected in body tissue and/or fluid via data from a wearable electromagnetic brain activity sensor. In an example, this system can decrease the frequency, regularity, magnitude, and/or coordination of heart muscle contractions via an implanted cardiac management device when high blood pressure levels are detected via data from a wearable electromagnetic brain activity sensor.

In an example, this system can adjust parameters of cardiac functioning in response to abnormal blood pressure levels detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, this system can comprise a (partially or fully) closed-loop system for automatic adjustment of cardiac functioning via an implanted cardiac management device based on data from an electromagnetic brain activity sensor. In an example, automatic adjustment of cardiac functioning in response to detection of abnormal biometric parameter values can help to restore underlying biological and/or physiological processes to their proper functioning. For example, detection of a high blood pressure levels by a wearable electromagnetic energy sensor can trigger decreased blood flow which, in turn, can help to lower blood pressure levels.

In an example, an electromagnetic brain activity sensor can be configured to collect data concerning electromagnetic brain activity which is associated with sleep and/or different stages of sleep. Sleep and/or different stages of sleep are associated with changes in electromagnetic brain activity. These changed patterns are detected by analysis of data from one or more wearable electromagnetic energy sensors. Based on this, the system can optimize cardiac function for sleep and/or for different stages of sleep. In an example, this system can adjust the frequency, regularity, magnitude, and/or coordination of heart muscle contractions via an implanted cardiac management device based on a person's sleep status and/or stage of sleep. In an example, this system can adjust parameters of cardiac functioning in response to sleep (and/or stage of sleep) as detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic brain activity sensor can be configured to collect data concerning electromagnetic brain activity which is affected by physical activity and/or exercise level. Physical activity and/or exercise level can trigger changes in electromagnetic brain activity. These changed patterns are detected by analysis of data from one or more wearable electromagnetic energy sensors. Based on this, the system can optimize cardiac function for physical activity and/or exercise level. In an example, this system can adjust the frequency, regularity, magnitude, and/or coordination of heart muscle contractions via an implanted cardiac management device based on a person's physical activity and/or exercise level. In an example, this system can adjust parameters of cardiac functioning in response to physical activity and/or exercise level as detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic brain activity sensor can be configured to collect data concerning electromagnetic brain activity which is affected by level of mental exertion or focus. Mental exertion or focus is associated with changes in electromagnetic brain activity. These changed patterns are detected by analysis of data from one or more wearable electromagnetic energy sensors. Based on this, the system can optimize cardiac function for level of mental exertion or focus. In an example, this system can adjust the frequency, regularity, magnitude, and/or coordination of heart muscle contractions via an implanted cardiac management device based on a person's level of mental exertion or focus. In an example, this system can adjust parameters of cardiac functioning in response to level of mental exertion or focus as detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic brain activity sensor can be configured to collect data concerning electromagnetic brain activity which is affected by stress level. Stress is associated with changes in electromagnetic brain activity. These changed patterns are detected by analysis of data from one or more wearable electromagnetic energy sensors. Based on this, the system can optimize cardiac function for stress level. In an example, this system can adjust the frequency, regularity, magnitude, and/or coordination of heart muscle contractions via an implanted cardiac management device based on a person's stress level. In an example, this system can adjust parameters of cardiac functioning in response to stress level as detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic brain activity sensor can be configured to collect data concerning electromagnetic brain activity which is affected by relaxation level. Relaxation is associated with changes in electromagnetic brain activity. These changed patterns are detected by analysis of data from one or more wearable electromagnetic energy sensors. Based on this, the system can optimize cardiac function for relaxation level. In an example, this system can adjust the frequency, regularity, magnitude, and/or coordination of heart muscle contractions via an implanted cardiac management device based on a person's relaxation level. In an example, this system can adjust parameters of cardiac functioning in response to relaxation level as detected by a wearable electromagnetic brain activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic brain activity sensor can be configured to collect data concerning one or more biometric parameters or conditions selected from the group consisting of: activity level, atrial fibrillation, bilirubin level, blood flow, blood oxygen (SpO2), blood pressure, bradycardia, breathing rate, calcium level, caloric intake, carbon dioxide level, carbon dioxide level, cardiopulmonary function, creatine level, eating behavior, electrolyte levels, emotional state, exercise level, eye movement, glucose level, glucose level, hormone level, hydration, hydration, hypertension, lactate level, magnesium level, oxygen saturation, pH level, potassium level, sleep or stage of sleep, speech, stress level, and troponin level.

In an example, this system can adjust parameters of cardiac functioning in response to abnormal values of one or more of these biometric parameters or conditions as detected by a wearable electromagnetic brain activity sensor. In an example, these cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic energy sensor can be an electromagnetic muscle activity sensor. In an example, an electromagnetic energy sensor can be an electromyographic (EMG) sensor. In an example, an electromagnetic muscle activity sensor can collect data concerning the natural emission of electromagnetic energy by a person's muscles and/or the nerves which innervate those muscles. In an example, the operation of an implanted cardiac management device can be adjusted based on detection of a selected pattern of electromagnetic neuromuscular activity.

In an example, an electromagnetic energy sensor can comprise an electromagnetic energy emitter and an electromagnetic energy receiver which are in proximity to a person's muscles. In an example, an electromagnetic energy sensor can collect data concerning changes in the transmission of electromagnetic energy from the emitter to the receiver due to changes in neuromuscular activity. In an example, an electromagnetic muscle activity sensor can measure voltage fluctuations resulting from neuromuscular activity. In an example, an electromagnetic energy sensor that collects data concerning neuromuscular activity can be a capacitive sensor or a conductive sensor. In an example, an electromagnetic energy sensor that collects data concerning neuromuscular activity can be a dry electrode or a wet electrode.

In an example, this system can include an electromyographic (EMG) sensor which is incorporated into an article of clothing or a clothing accessory. In an example, this system can include a plurality of electromyographic (EMG) sensors which are incorporated into an article of clothing or a clothing accessory. A plurality of electromyographic (EMG) sensors can provide more accurate measurement of whole-body activity level than a similarly-placed plurality of motion sensors because electromyographic sensors can measure isometric exertion. In an example, the operation of an implanted cardiac management device can be adjusted based on whole-body activity level as measured by a plurality of electromyographic (EMG) sensors.

In an example, this system can adjust parameters of cardiac functioning in response to a variation in whole-body activity level which is detected by an electromagnetic muscle activity sensor or array of electromagnetic muscle activity sensors. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, a plurality of electromagnetic muscle activity sensors can be configured to collect data concerning electromagnetic neuromuscular activity. Whole-body activity triggers changes the patterns of electromagnetic neuromuscular activity measured by these sensors. These changed patterns trigger adjustment of cardiac function via the implanted cardiac management device which, in turn, adjusts cardiac function to optimally match cardiac function to whole-body activity level.

In an example, this system can increase the frequency of a heart beats via an implanted cardiac management device when a high whole-body activity level is detected via data from an electromagnetic muscle activity sensor. In an example, this system can increase the magnitude of heart contractions via an implanted cardiac management device when a high whole-body activity level is detected via data from an electromagnetic muscle activity sensor. In an example, this system can increase the frequency, regularity, magnitude, and/or coordination of heart muscle contractions via an implanted cardiac management device when a high whole-body activity level is detected via data from an electromagnetic muscle activity sensor.

In an example, this system can decrease the frequency of a heart beats via an implanted cardiac management device when a low whole-body activity level is detected via data from an electromagnetic muscle activity sensor. In an example, this system can decrease the magnitude of heart contractions via an implanted cardiac management device when a low whole-body activity level is detected via data from an electromagnetic muscle activity sensor. In an example, this system can decrease the frequency, regularity, magnitude, and/or coordination of heart muscle contractions via an implanted cardiac management device when a low whole-body activity level is detected via data from an electromagnetic muscle activity sensor.

In an example, this system can adjust parameters of cardiac functioning in response to a high or low whole-body activity level detected by one or more electromagnetic muscle activity sensors (such as EMG sensors). These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, an electromagnetic muscle activity sensor can be configured to collect data concerning electromagnetic neuromuscular activity which is affected by muscle tissue lactate (and/or lactic acid) levels or changes in those levels. In an example, abnormal muscle tissue lactate (and/or lactic acid) levels or changes in those levels can trigger changes in patterns of electromagnetic neuromuscular activity. These changed patterns can be detected by analysis of data from one or more wearable electromagnetic energy sensors, which in turn triggers adjustment of cardiac function via the implanted cardiac management device which, in turn, restores normal muscle tissue lactate (and/or lactic acid) levels.

In an example, this system can increase the frequency of a heart beats via an implanted cardiac management device when high lactate (and/or lactic acid) levels are detected via data from an electromagnetic muscle activity sensor. In an example, this system can increase the magnitude of heart contractions via an implanted cardiac management device when high lactate (and/or lactic acid) levels are detected via data from an electromagnetic muscle activity sensor. In an example, this system can increase the frequency, regularity, magnitude, and/or coordination of heart muscle contractions via an implanted cardiac management device when high lactate (and/or lactic acid) levels are detected via data from an electromagnetic muscle activity sensor.

In an example, this system can adjust parameters of cardiac functioning in response to high lactate (and/or lactic acid) levels detected by an electromagnetic muscle activity sensor. These cardiac functioning parameters can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, this system can adjust a person's cardiac function based on their whole-body posture and/or configuration. In an example, this system can adjust a person's cardiac function based on identification of a specific whole-body posture and/or configuration. In an example, this system can adjust a person's cardiac function based on identification of a specific type of activity based on measured whole-body posture and/or configuration. In an example, this system can increase (or decrease) the frequency of a person's heart beats and/or the magnitude of a person's heart contractions in response to a change in the person's whole-body posture and/or configuration as detected by one or more wearable biometric sensors. In an example, a person's whole-body posture and/or configuration can be measured by one or more motion sensors, electromyographic (EMG sensors), and/or bend sensors.

In an example, the wearable component of this system can include one or more body motion and/or configuration sensors. In an example, these one or more body motion and/or configuration sensors can be used to identify whether the person is engaged in one or more selected types of physical activities. In an example, these one or more body motion and/or configuration sensors can be used to identify whether the person is: walking or running; engaging in a particular type of exercise; playing a particular type of sport; eating; and/or sleeping. In an example a body motion and/or configuration sensor can be selected from the group consisting of: accelerometer, altimeter, electrogoniometer, electrogoniometer, electromyographic (EMG) sensor, GPS sensor, gyroscope, inclinometer, motion sensor, pressure sensor, stretch sensor, and strain gauge. In an example, this device can comprise one or more motion sensors. One or more motion sensors can be selected from the group consisting of: accelerometer, gyroscope, inclinometer, strain sensor, stretch sensor, and electrogoniometer. In an example this system can comprise a plurality and/or array of body motion and/or configuration sensors which are selected from the group consisting of: accelerometer, altimeter, electrogoniometer, electrogoniometer, electromyographic (EMG) sensor, GPS sensor, gyroscope, inclinometer, motion sensor, pressure sensor, stretch sensor, and strain gauge.

In an example, this system can adjust a person's cardiac function based on their respiration rate. In an example, this system can increase (or decrease) the frequency of a person's heart beats and/or the magnitude of a person's heart contractions in response to an increase (or decrease) in the person's respiration rate as detected by a wearable biometric sensor. In an example, a person's respiration rate can be measured by reflecting light energy from (and/or passing light energy through) body tissue. In an example, a person's respiration rate can be measured by measuring electromagnetic energy from (and/or passing electromagnetic energy through) body tissue.

In an example, this system can adjust a person's cardiac function based on their skin moisture level. In an example, this system can increase (or decrease) the frequency of a person's heart beats and/or the magnitude of a person's heart contractions in response to an increase (or decrease) in the person's skin moisture level as detected by a wearable biometric sensor. In an example, a person's skin moisture level can be measured by reflecting light energy from (and/or passing light energy through) body tissue. In an example, a person's skin moisture level can be measured by measuring electromagnetic energy from (and/or passing electromagnetic energy through) body tissue.

In an example, this system can adjust a person's cardiac function based on their tissue impedance level. In an example, this system can increase (or decrease) the frequency of a person's heart beats and/or the magnitude of a person's heart contractions in response to an increase (or decrease) in the person's tissue impedance level as detected by a wearable biometric sensor. In an example, a person's tissue impedance level can be measured by reflecting light energy from (and/or passing light energy through) body tissue. In an example, a person's tissue impedance level can be measured by measuring electromagnetic energy from (and/or passing electromagnetic energy through) body tissue.

In an example, this system can include one or more biochemical and/or biologic sensors selected from the group consisting of:, amino acid sensor, antibody-based receptor, artificial olfactory sensor, artificial taste bud, biochemical sensor, biological cell sensor, biological sensor, biomimetic sensor, chemical sensor, chemiresistor, chemoreceptor, cholesterol sensor, DNA-based sensor, electrochemical sensor, electronic nose, electroosmotic sensor, electrophoresis sensor, electroporation sensor, enzyme-based sensor, fat sensor, glucose sensor, HDL sensor, LDL sensor, membrane sensor, micronutrient sensor, microorganism-based sensor, multiple-analyte sensor array, nucleic acid-based sensor, olfactory sensor, osmolality sensor, pH level sensor, plurality of cross-reactive sensors, protein-based sensor, reagent-based sensor, receptor-based sensor, RNA-based sensor, saturated fat sensor, sodium sensor, and trans fat sensor.

In an example, this system can comprise one or more sensors selected from the group consisting of: accelerometer, acoustic energy sensor, action potential sensor, activity level sensor, auscultatory sensor, ballistocardiographic sensor, bend sensor, biochemical sensor, blood flow sensor, blood pressure sensor, brain activity sensor, breathing rate sensor, caloric intake monitor, capacitance hygrometry sensor, capacitive sensor, cardiac function sensor, cardiopulmonary function sensor, chemiluminescence sensor, chemoreceptor, chewing sensor, chromatographic sensor, compass, conductivity sensor, core temperature sensor, cranial pressure sensor, digital camera, electrical resistance sensor, electrocardiographic (ECG) sensor, electroencephalographic (EEG) sensor, electrogoniometer, electromagnetic energy sensor, electromyographic (EMG) sensor, electroporation sensor, enzymatic sensor, eye muscle (EOG) sensor, galvanic skin response (GSR) sensor, glucose sensor, GPS sensor, gyroscope, Hall-effect sensor, heart rate monitor, heart rate sensor, hormone sensor, humidity sensor, hydration level sensor, hygrometry sensor, impedance sensor, inclinometer, inertial sensor, infrared light (IR) sensor, infrared spectroscopy sensor, ion mobility spectroscopic sensor, lactate sensor, laser sensor, light intensity sensor, magnetic energy sensor, magnetometer, medichip, and metal oxide semiconductor sensor.

In an example, this system can comprise one or more sensors selected from the group consisting of: Micro Electrical Mechanical System (MEMS) sensor, microcantilever sensor, microfluidic sensor, microphone, motion sensor, muscle function monitor, near-infrared spectroscopic sensor, neural impulse monitor, neurosensor, optical detector, optical sensor, optoelectronic sensor, oximetry sensor, perspiration rate sensor, pH level sensor, photochemical sensor, photodetector, photodiode, photoelectric sensor, photoplethysmographic (PPG) sensor, piezocapacitive sensor, piezoelectric sensor, piezoresistive sensor, position sensor, pressure sensor, pulse oximetry sensor, pulse rate sensor, pyroelectric sensor, radio frequency (RF) sensor, Raman spectroscopy sensor, respiration sensor, skin conductance sensor, skin moisture sensor, skin temperature sensor, sound energy sensor, spectral analysis sensor, spectrometric sensor, spectrophotometer, spectroscopic sensor, still-frame camera, strain gauge, stretch sensor, swallowing sensor, sweat sensor, systolic blood pressure sensor, temperature sensor, thermal energy sensor, thermistor, thermocouple, thermometer, thermopile, tissue impedance sensor, ultrasonic energy sensor, ultraviolet light sensor, ultraviolet spectroscopy sensor, variable impedance sensor, variable resistance sensor, variable translucence sensor, and video camera.

In an example, this device can further comprise one or more environmental sensors. In an example, the operation of this device can be automatically adjusted, modified, and/or controlled based on data from one or more environmental sensors. In an example, it can be advantageous for optimal operation of an implanted cardiac function device to be different in different environmental settings and/or conditions. In an example, the operation of an implanted cardiac function device is advantageously adjusted based on environmental (and/or ambient) temperature, humidity, altitude, and barometric pressure. In an example, the operation of an implanted cardiac function device is advantageously adjusted based on environmental (and/or ambient) light level, spectral distribution, and/or variability. In an example, the operation of an implanted cardiac function device is advantageously adjusted based on ambient noise level, spectral distribution, variability, sound pattern recognition, ambient voices, and ambient ultrasonic energy.

In an example, the operation of an implanted cardiac function device can be adjusted based on ambient air composition, air quality, oxygen level, carbon dioxide level, carbon monoxide level, air-borne pollution and/or toxins, air borne allergens, and air speed. In an example, the operation of an implanted cardiac function device can be adjusted based on environmental (and/or ambient) electromagnetic radiation levels and/or types. In an example, the operation of an implanted cardiac function device can be adjusted based on whether a person is indoors or outdoors. In an example, this device can further comprise one or more environmental and/or ambient sensors selected from the group consisting of: air-borne allergen sensor, air-borne pollution sensor, air-borne toxin sensor, altitude sensor, ambient air composition sensor, ambient air quality sensor, ambient carbon dioxide sensor, ambient carbon monoxide sensor, ambient electromagnetic radiation sensor, ambient humidity sensor, ambient light sensor, ambient noise sensor, ambient oxygen sensor, ambient sound pattern recognition sensor, ambient temperature sensor, ambient ultrasonic energy sensor, ambient voices sensor, and barometric pressure sensor.

In an example, this system can comprise one or more data processing components selected from the group consisting of: data processor, data receiver, data transmitter, memory, microchip, and microprocessor. In an example, this system can include a wireless data transmitter and/or receiver. In an example, a first data processor and/or data transmitter which is physically part of the wearable component can be in electronic communication with a second data processor and/or data receiver which is not physically part of the wearable component. In an example, data processing can be distributed between the first and second data processors. In an example, a second data processor can be part of a remote computing device. In an example, a second data processor can be part of a wearable data processing hub, mobile computer, electronic tablet, electronic pad, mobile phone, smart phone, implanted medical device, internet-connected remote computer, communication network tower, satellite, or home control system.

In an example, this system can comprise one or more power sources which supply power to the biometric sensor and the data processor. In an example, a power source can be a battery. In an example, a power source and/or power transducer can transduce, harvest, and/or generate energy from body motion or kinetic energy. In an example, a power source and/or power transducer can transduce, harvest, and/or generate energy from ambient light energy. In an example, a power source and/or power transducer can transduce, harvest, and/or generate energy from body thermal energy. In an example, a power source and/or power transducer can transduce, harvest, and/or generate energy from ambient electromagnetic energy.

In an example, this system can further comprise one or more human-to-computer-interface (HCI) components. One or more human-computer-interface components can be selected from the group consisting of: touch screen, gesture recognition interface, speech and/or voice recognition interface, button and/or keypad, dial and/or knob, brainwave-based HCI, and motion sensor. In an example, this device can further comprise one or more computer-to-human interface (HCI) components. One or more computer-to-human interface components can be selected from the group consisting of: display screen, light emitter and/or light-emitting array, light-emitting fabric, optical emitter, speaker, buzzer, or other sound-emitting member, electromagnetic signal generator, vibrating member, actuator, Micro Electro Mechanical Systems (MEMS), augmented reality eyewear, virtual reality eyewear, and electronically-functional eyewear.

In an example, this invention can be embodied in an integrated system for managing cardiac rhythm including both a wearable device and an implanted device, wherein this system comprises: (a) a wearable device which is configured to be worn by a person, wherein the wearable device further comprises a light emitter which is configured to emit light toward the person's body tissue, a light receiver which is configured to receive light from the light emitter after the light has passed through and/or been reflected from the person's body tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's body tissue; and (d) wherein the operation of the cardiac rhythm management device is changed based on the amount of the analyte in the person's body tissue.

In an example, the wearable component of the system can be a finger ring. In an example, this invention can be embodied in an integrated system for managing cardiac rhythm including both a wearable device and an implanted device, wherein this system comprises: (a) a finger ring which is configured to be worn by a person, wherein the finger ring further comprises a light emitter which is configured to emit light toward the person's finger tissue, a light receiver which is configured to receive light from the light emitter after the light has passed through and/or been reflected from the person's finger tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's finger tissue; and (d) wherein the operation of the cardiac rhythm management device is changed based on the amount of the analyte in the person's finger tissue.

In an example, a finger ring can have a circular cross-sectional shape. In an example, a finger ring can have a circular circumference. In an example, a finger ring can have a width which is perpendicular to its circular circumference. In an example, the average width of a finger ring can be in the range of ⅛ inch to 1 inch. In an example, the average width of a finger ring can be in the range of 3 mm to 3 cm. In an example, a finger ring can have an inward side which is configured to face toward the surface of a person's finger and an outward side which is configured to face away from the surface of a person's finger. In an example, the inward side of a finger ring can be flat. In an example, the inward side of a ringer ring can be rounded.

In an example, the wearable component of the system can be a wrist and/or arm band. In an example, this invention can be embodied in an integrated system for managing cardiac rhythm including both a wrist and/or arm band and an implanted device, wherein this system comprises: (a) a wrist and/or arm band which is configured to be worn by a person, wherein the wrist and/or arm band further comprises a light emitter which is configured to emit light toward the person's wrist and/or arm tissue, a light receiver which is configured to receive light from the light emitter after the light has passed through and/or been reflected from the person's wrist and/or arm tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's wrist and/or arm tissue; and (d) wherein the operation of the cardiac rhythm management device is changed based on the amount of the analyte in the person's wrist and/or arm tissue.

In an example, the wearable component of the system can be an ear ring, ear insert, or other ear-worn device. In an example, this invention can be embodied in an integrated system for managing cardiac rhythm including both an ear ring, ear insert, or other ear-worn device and implanted device, wherein this system comprises: (a) an ear ring, ear insert, or other ear-worn device which is configured to be worn by a person, wherein the ear ring, ear insert, or other ear-worn device further comprises a light emitter which is configured to emit light toward the person's ear tissue, a light receiver which is configured to receive light from the light emitter after the light has passed through and/or been reflected from the person's ear tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's ear tissue; and (d) wherein the operation of the cardiac rhythm management device is changed based on the amount of the analyte in the person's ear tissue.

In an example, the analyte measured by this device can be the oxygen level of body tissue (and/or fluid). In an example, the system can change the frequency and/or magnitude of electromagnetic pulses delivered to a person's heart when analysis of data from the light receiver indicates a change in the level of oxygen in body tissue (and/or fluid). In an example, the system can increase the frequency and/or magnitude of electromagnetic pulses delivered to the person's heart when analysis of data from the light receiver indicates a low level of oxygen in body tissue (and/or fluid). In an example, the system can decrease the frequency and/or magnitude of electromagnetic pulses delivered to the person's heart when analysis of data from the light receiver indicates a high level of oxygen in body tissue (and/or fluid).

In an example, a light emitter can emit light from the inward side of a wearable device toward the surface of a person's body (e.g. finger, wrist, arm, ear, or leg). In an example, a light receiver can receive light into the inward side of a wearable device which has passed through and/or been reflected from a person's body tissue. In an example, there can be a flexible and/or compressible light barrier between a light emitter and a light receiver. In an example, a light emitter and a light receiver can be on the same circumferential line (e.g. circle) of a wearable device, but at different radial locations around this circumference. In an example, a light emitter and a light receiver can be on the same radial location around a wearable device, but on different circumferential lines (e.g. circles). In an example, there can be two or more light emitters and one light receiver on a wearable device. In an example, there is one light emitter and two or more light receivers on a wearable device.

In an example, compass coordinates can be defined for the circumference of a wearable device with the 0-degree point being the most ventral point when the wearable device is worn, the 90-degree point being one-quarter of the way around the circumference in a clockwise direction from the 0-degree point, the 180-degree point being opposite the 0-degree point, and the 270-degree point being one-quarter of the way around the circumference in a clockwise direction from the 180-degree point. In an example, a light emitter can be separated from a light receiver by between 1 and 15 degrees. In an example, a light emitter can be separated from a light receiver by between 10 and 45 degrees. In an example, a light emitter can be separated from a light receiver by more than 44 degrees. In an example, a light emitter can be separated from a light receiver by 45, 60, 90, or 180 degrees. In an example, a plurality of light receivers can be distributed around the circumference of a wearable device, being pair-wise separated from each other by between 10 and 45 degrees. In an example, a plurality of light receivers can be distributed around the circumference of a wearable device, being pair-wise separated from each other by 45, 60, 90, or 180 degrees.

In an example, a light emitter can emit coherent light. In an example, a light emitter can be a laser. In an example, a light emitter can be a Light Emitting Diode (LED). In an example, a light emitter can emit infrared or near-infrared light. In an example, a light emitter can emit ultraviolet light. In an example, a light emitter emit red light and/or be a red-light laser. In an example, a light emitter emit green light and/or be a green-light laser. In an example, a light emitter can emit white light and/or be a white-light laser. In an example, a wearable device can include can be two or more light emitters. In an example, a wearable device can include a red light emitter and a green light emitter. In an example, a light emitter can emit light with frequency and/or spectrum changes over time. In an example, a light emitter can emit a sequence of light pulses at different selected frequencies. In an example, a light emitter can emit polarized light. In an example, the polarization of light can change after the light passes through and/or is reflected from body tissue and these changes can be used to measure an analyte level in the body.

In an example, differences in the spectrum light emitted from a light emitter and the spectrum of light received by a light receiver can be analyzed using spectroscopic analysis. In an example, changes in the amount (or concentration) of a selected analyte in body tissue can change the spectrum of light passing through and/or reflected by the body tissue. In an example, body tissue can be understood to include fluids such as blood and interstitial fluid. In an example, a light emitter and a light received can be collectively referred to as a spectroscopic (or spectroscopy) sensor. In an example, the analyte which is measured can be oxygen. In example, differences in the spectrum of light emitted from a light emitter and the spectrum of light received by a light receiver can be analyzed to measure tissue (and/or blood) oxygen levels. In example, differences in the spectrum of light emitted from a light emitter and the spectrum of light received by a light receiver can be analyzed to measure tissue (and/or blood) oxygenation.

In an example, a light emitter and a light receiver together can comprise a spectroscopic (or “spectroscopy”) sensor. In an example, the spectrum of light energy is changed when the light energy passes through body tissue and/or is reflected from body tissue. In an example, changes in the spectrum of light energy which has passed through and/or been reflected from body tissue can be analyzed to detect the composition and/or configuration of body tissue. In an example, these changes in the spectrum of light energy can be analyzed to provide information on the composition of body tissue which, in turn, enables measurement of an analyte level in the body. In an example, a light emitter and a light receiver together can comprise a sensor selected from the group consisting of: backscattering spectrometry sensor, infrared spectroscopy sensor, ion mobility spectroscopic sensor, mass spectrometry sensor, Near Infrared Spectroscopy sensor (NIS), Raman spectroscopy sensor, spectrometry sensor, spectrophotometer, spectroscopy sensor, ultraviolet spectroscopy sensor, and white light spectroscopy sensor.

In an example, portions of the spectrum of light emitted by a light emitter can be absorbed by body tissue and spectral analysis of these absorbed portions can enable measurement of an analyte level in the body. In an example, portions of the spectrum of light emitted by a light emitter can be amplified by body tissue and spectral analysis of these amplified portions can enable measurement of an analyte level in the body. In an example, portions of the spectrum of light emitted by a light emitter can be shifted by interaction with body tissue and spectral analysis of these shifted portions can enable measurement of an analyte level in the body.

In an example, the depth, breadth, location, and/or type of body tissue or fluid from which light from a light emitter is reflected can be changed by adjusting the frequency, color, and/or spectrum of light emitted from the light emitter. In an example, the frequency, color, and/or spectrum of light emitted from the light emitter can be adjusted in order to more accurately measure an analyte level in the body. In an example, the frequency, color, and/or spectrum of light emitted from the light emitter can be adjusted automatically (in an iterative manner) by a device in order to more accurately measure an analyte level in the body for a specific person, for a specific type of activity, or for a specific configuration of the device relative to the person's body surface. In an example, the frequency, color, and/or spectrum of light emitted from the light emitter can be adjusted automatically to maintain accurate measurement of an analyte level in the body even if the device shifts and/or moves relative to the person's body surface. In an example, a device can automatically vary the frequency, color, and/or spectrum of light from a light emitter to scan through a range of tissue depths, locations, and/or types in order to obtain more accurate measurement of an analyte level in the body. In an example, this device can further comprise one or more optical filters or lenses which change the frequency, color, and/or spectrum of light emitted by a light emitter.

In an example, the depth, breadth, location, and/or type of body tissue or fluid from which light from a light emitter is reflected can be changed by adjusting the power and/or intensity of light emitted from the light emitter. In an example, the power and/or intensity of light emitted from the light emitter can be adjusted in order to more accurately measure an analyte level in the body. In an example, the power and/or intensity of light emitted from the light emitter can be adjusted automatically (in an iterative manner) by a device in order to more accurately measure an analyte level in the body for a specific person, for a specific type of activity, or for a specific configuration of the device relative to the person's body surface. In an example, the power and/or intensity of light emitted from the light emitter can be adjusted automatically to maintain accurate measurement of an analyte level in the body even if the device shifts and/or moves relative to the person's body surface. In an example, a device can automatically vary the power and/or intensity of light from a light emitter to scan through a range of tissue depths, locations, and/or types in order to obtain more accurate measurement of an analyte level in the body.

In an example, the depth, breadth, location, and/or type of body tissue or fluid from which light from a light emitter is reflected can be changed by adjusting the angle of light emitted from the light emitter. In an example, the angle of light emitted from the light emitter can be adjusted in order to more accurately measure an analyte level in the body. In an example, the angle of light emitted from the light emitter can be adjusted automatically (in an iterative manner) by a device in order to more accurately measure an analyte level in the body for a specific person, for a specific type of activity, or for a specific configuration of the device relative to the person's body surface. In an example, the angle of light emitted from the light emitter can be adjusted automatically to maintain accurate measurement of an analyte level in the body even if the device shifts and/or moves relative to the person's body surface. In an example, a device can automatically vary the angle of light from a light emitter to scan through a range of tissue depths, locations, and/or types in order to obtain more accurate measurement of an analyte level in the body. In an example, this device can further comprise one or more optical filters or lenses which change the projection and/or body incidence angle of a light beam emitted by a light emitter.

In an example, the depth, breadth, location, and/or type of body tissue or fluid from which light from a light emitter is reflected can be changed by adjusting the coherence, polarization, and/or phase of light emitted from the light emitter. In an example, the coherence, polarization, and/or phase of light emitted from the light emitter can be adjusted in order to more accurately measure an analyte level in the body. In an example, the coherence, polarization, and/or phase of light emitted from the light emitter can be adjusted automatically (in an iterative manner) by a device in order to more accurately measure an analyte level in the body for a specific person, for a specific type of activity, or for a specific configuration of the device relative to the person's body surface. In an example, the coherence, polarization, and/or phase of light emitted from the light emitter can be adjusted automatically to maintain accurate measurement of an analyte level in the body even if the device shifts and/or moves relative to the person's body surface. In an example, a device can automatically vary the coherence, polarization, and/or phase of light from a light emitter to scan through a range of tissue depths, locations, and/or types in order to obtain more accurate measurement of an analyte level in the body. In an example, this device can further comprise one or more optical filters or lenses which change the coherence, polarization, and/or phase of light emitted by a light emitter.

In an example, a device can comprise a first light emitter and a second light emitter. In an example, the first light emitter can emit light with a first light frequency, color, and/or spectrum and the second light emitter can emit light with a second light frequency, color, and/or spectrum. In an example, light from the first light emitter can reflect primarily from a first depth, breadth, location, and/or type of body tissue and light from the second light emitter can reflect primarily from a first depth, breadth, location, and/or type of body tissue. In an example, first and second light emitters can emit light simultaneously. In an example, first and second light emitters can emit light in a selected chronological sequence and/or timing pattern.

In an example, a device can comprise a first light emitter and a second light emitter. In an example, the first light emitter can emit light with a first light power and/or intensity and the second light emitter can emit light with a second light power and/or intensity. In an example, light from the first light emitter can reflect primarily from a first depth, breadth, location, and/or type of body tissue and light from the second light emitter can reflect primarily from a first depth, breadth, location, and/or type of body tissue. In an example, first and second light emitters can emit light simultaneously. In an example, first and second light emitters can emit light in a selected chronological sequence and/or timing pattern.

In an example, a device can comprise a first light emitter and a second light emitter. In an example, the first light emitter can emit light with a first light projection and/or body incidence angle and the second light emitter can emit light with a second light projection and/or body incidence angle. In an example, light from the first light emitter can reflect primarily from a first depth, breadth, location, and/or type of body tissue and light from the second light emitter can reflect primarily from a first depth, breadth, location, and/or type of body tissue. In an example, first and second light emitters can emit light simultaneously. In an example, first and second light emitters can emit light in a selected chronological sequence and/or timing pattern.

In an example, a device can comprise a first light emitter and a second light emitter. In an example, the first light emitter can emit light with a first light coherence, polarization, and/or phase and the second light emitter can emit light with a second light coherence, polarization, and/or phase. In an example, light from the first light emitter can reflect primarily from a first depth, breadth, location, and/or type of body tissue and light from the second light emitter can reflect primarily from a first depth, breadth, location, and/or type of body tissue. In an example, first and second light emitters can emit light simultaneously. In an example, first and second light emitters can emit light in a selected chronological sequence and/or timing pattern.

In an example, the depth, breadth, location, and/or type of body tissue or fluid from which light from a light emitter is reflected and received by a light receiver can be changed by adjusting the distance between a light emitter and a light receiver. In an example, the distance between a light emitter and a light receiver can be adjusted in order to more accurately measure an analyte level in the body. In an example, the distance between a light emitter and a light receiver can be adjusted automatically (in an iterative manner) by a device in order to more accurately measure an analyte level in the body for a specific person, for a specific type of activity, or for a specific configuration of the device relative to the person's body surface. In an example, the distance between a light emitter and a light receiver can be adjusted automatically to maintain accurate measurement of an analyte level in the body even if the device shifts and/or moves relative to the person's body surface. In an example, a device can automatically vary the distance between a light emitter and a light receiver to scan through a range of tissue depths, locations, and/or types in order to obtain more accurate measurement of an analyte level in the body.

In an example, the depths, breadths, locations, and/or types of body tissue or fluid from which light beams from a plurality of light emitters are reflected can be determined by a selected geometric configuration of the plurality of light emitters and a light receiver. In an example, a selected geometric configuration of a plurality of light emitters and a light receiver can be designed to most accurately measure an analyte level in the body. In an example, the geometric configuration of a plurality of light emitters and a light receiver can be adjusted automatically (in an iterative manner) by a device in order to more accurately measure an analyte level in the body for a specific person, for a specific type of activity, or for a specific configuration of the device relative to the person's body surface. In an example, the geometric configuration of a plurality of light emitters and a light receiver can be adjusted automatically to maintain accurate measurement of an analyte level in the body even if the device shifts and/or moves relative to the person's body surface. In an example, a device can automatically vary the geometric configuration of a plurality of light emitters and a light receiver in order to scan through a range of tissue depths, locations, and/or types in order to measure an analyte level in the body more accurately. In an example, a plurality of light emitters can emit light simultaneously. In an example, a plurality of light emitters can emit light in a selected chronological sequence and/or timing pattern.

In an example, a plurality of light emitters can be configured in a linear array in proximity to a light receiver. In an example, a plurality of light emitters can be configured in a linear array including a light receiver. In an example, a plurality of light emitters can be configured in a polygonal array in proximity to a light receiver. In an example, a plurality of light emitters can be configured in a polygonal array including a light receiver. In an example, a plurality of light emitters can be configured in a polygonal array around a light receiver. In an example, a plurality of light emitters can be configured in a circular or other arcuate array in proximity to a light receiver. In an example, a plurality of light emitters can be configured in a circular or other arcuate array including a light receiver. In an example, a plurality of light emitters can be configured in a circular or other arcuate array around a light receiver. In an example, a plurality of light emitters can emit light in a circular sequence around a central light receiver.

In an example, the depths, breadths, locations, and/or types of body tissue or fluid from which light beams are reflected and received by a plurality of light receivers can be determined by a selected geometric configuration of a light emitter and the plurality of light receivers. In an example, a selected geometric configuration of a light emitter and a plurality of light receivers can be designed to most accurately measure an analyte level in the body. In an example, the geometric configuration of a light emitter and a plurality of light receivers can be adjusted automatically (in an iterative manner) by a device in order to more accurately measure an analyte level in the body for a specific person, for a specific type of activity, or for a specific configuration of the device relative to the person's body surface. In an example, the geometric configuration of a light emitter and a plurality of light receivers can be adjusted automatically to maintain accurate measurement of an analyte level in the body even if the device shifts and/or moves relative to the person's body surface. In an example, a device can automatically vary the geometric configuration of a light emitter and a plurality of light receivers in order to scan through a range of tissue depths, locations, and/or types in order to measure an analyte level in the body more accurately.

In an example, a plurality of light receivers can be configured in a linear array in proximity to a light emitter. In an example, a plurality of light receivers can be configured in a linear array including a light emitter. In an example, a plurality of light receivers can be configured in a polygonal array in proximity to a light emitter. In an example, a plurality of light receivers can be configured in a polygonal array including a light emitter. In an example, a plurality of light receivers can be configured in a polygonal array around a light emitter. In an example, a plurality of light receivers can be configured in a circular or other arcuate array in proximity to a light emitter. In an example, a plurality of light receivers can be configured in a circular or other arcuate array including a light emitter. In an example, a plurality of light receivers can be configured in a circular or other arcuate array around a light emitter.

In an example, a light emitter can be part of an arcuate band. In an example, a light emitter can be part of a housing which is held on a person's body by an arcuate band. In an example, this device can comprise an array, grid, and/or matrix of two or more light emitters with a proximal-to-distal orientation. In an example, this device can comprise an array, grid, and/or matrix of two or more light emitters along a proximal-to-distal axis. In an example, this device can comprise an array, grid, and/or matrix of two or more light emitters with a circumferential orientation. In an example, this device can comprise an array, grid, and/or matrix of two or more light emitters along a circumferential axis.

In an example, this device can comprise a linear array, grid, and/or matrix of light emitters. In an example, this device can comprise a rectangular array, grid, and/or matrix of light emitters. In an example, this device can comprise a circular or elliptical array, grid, and/or matrix of light emitters. In an example, this device can comprise a checkerboard array, grid, and/or matrix of light emitters. In an example, this device can comprise a three-dimensional stacked array, grid, and/or matrix of light emitters. In an example, this device can comprise a sunburst and/or radial-spoke array, grid, and/or matrix of light emitters. In an example, this device can comprise a sinusoidal array, grid, and/or matrix of light emitters.

In an example, an array, grid, and/or matrix of two or more light emitters can span up to 10% of the cross-sectional circumference of a part of a person's body such as a wrist, arm, finger, ankle, or leg. In an example, an array, grid, and/or matrix of two or more light emitters can span between 10% and 25% of the cross-sectional circumference of a part of a person's body such as a wrist, arm, finger, ankle, or leg. In an example, an array, grid, and/or matrix of two or more light emitters can span between 25% and 50% of the cross-sectional circumference of a part of a person's body such as a wrist, arm, finger, ankle, or leg. In an example, an array, grid, and/or matrix of two or more light emitters can span between 50% and 100% of the cross-sectional circumference of a part of a person's body such as a wrist, arm, finger, ankle, or leg.

In an example, a light receiver can be part of an arcuate band. In an example, a light receiver can be part of a housing which is held on a person's body by an arcuate band. In an example, this device can comprise an array, grid, and/or matrix of two or more light receivers with a proximal-to-distal orientation. In an example, this device can comprise an array, grid, and/or matrix of two or more light receivers along a proximal-to-distal axis. In an example, this device can comprise an array, grid, and/or matrix of two or more light receivers with a circumferential orientation. In an example, this device can comprise an array, grid, and/or matrix of two or more light receivers along a circumferential axis.

In an example, this device can comprise a linear array, grid, and/or matrix of light receivers. In an example, this device can comprise a rectangular array, grid, and/or matrix of light receivers. In an example, this device can comprise a circular or elliptical array, grid, and/or matrix of light receivers. In an example, this device can comprise a checkerboard array, grid, and/or matrix of light receivers. In an example, this device can comprise a three-dimensional stacked array, grid, and/or matrix of light receivers. In an example, this device can comprise a sunburst and/or radial-spoke array, grid, and/or matrix of light receivers. In an example, this device can comprise a sinusoidal array, grid, and/or matrix of light receivers.

In an example, an array, grid, and/or matrix of two or more light receivers can span up to 10% of the cross-sectional circumference of a part of a person's body such as a wrist, arm, finger, ankle, or leg. In an example, an array, grid, and/or matrix of two or more light receivers can span between 10% and 25% of the cross-sectional circumference of a part of a person's body such as a wrist, arm, finger, ankle, or leg. In an example, an array, grid, and/or matrix of two or more light receivers can span between 25% and 50% of the cross-sectional circumference of a part of a person's body such as a wrist, arm, finger, ankle, or leg. In an example, an array, grid, and/or matrix of two or more light receivers can span between 50% and 100% of the cross-sectional circumference of a part of a person's body such as a wrist, arm, finger, ankle, or leg.

In an example, a light emitter and a light receiver can be part of an arcuate band. In an example, a light emitter and a light receiver can be part of a housing which is held on a person's body by an arcuate band. In an example, this device can comprise an array, grid, and/or matrix of (alternating) light emitters and receivers with a proximal-to-distal orientation. In an example, this device can comprise an array, grid, and/or matrix of (alternating) light emitters and receivers along a proximal-to-distal axis. In an example, this device can comprise an array, grid, and/or matrix of (alternating) light emitters and receivers with a circumferential orientation. In an example, this device can comprise an array, grid, and/or matrix of (alternating) light emitters and receivers along a circumferential axis.

In an example, this device can comprise a linear array, grid, and/or matrix of (alternating) light emitters and receivers. In an example, this device can comprise a rectangular array, grid, and/or matrix of (alternating) light emitters and receivers. In an example, this device can comprise a circular or elliptical array, grid, and/or matrix of (alternating) light emitters and receivers. In an example, this device can comprise a checkerboard array, grid, and/or matrix of (alternating) light emitters and receivers. In an example, this device can comprise a three-dimensional stacked array, grid, and/or matrix of (alternating) light emitters and receivers. In an example, this device can comprise a sunburst and/or radial-spoke array, grid, and/or matrix of (alternating) light emitters and receivers. In an example, this device can comprise a sinusoidal array, grid, and/or matrix of (alternating) light emitters and receivers.

In an example, an array, grid, and/or matrix of (alternating) light emitters and receivers can span up to 10% of the circumference of a part of a person's body such as a wrist, arm, finger, ankle, or leg. In an example, an array, grid, and/or matrix of (alternating) light emitters and receivers can span between 10% and 25% of the circumference of a part of a person's body such as a wrist, arm, finger, ankle, or leg. In an example, an array, grid, and/or matrix of (alternating) light emitters and receivers can span between 25% and 50% of the circumference of a part of a person's body such as a wrist, arm, finger, ankle, or leg. In an example, an array, grid, and/or matrix of (alternating) light emitters and receivers can span between 50% and 100% of the circumference of a part of a person's body such as a wrist, arm, finger, ankle, or leg.

In an example, this device can comprise an array, grid, and/or matrix of light emitters which differ in one or more parameters selected from the group consisting of: location and/or distance from a light receiver; distance to body surface; light beam frequency, color, and/or spectrum; light beam coherence, polarity, and/or phase; light beam power and/or intensity; light beam projection and/or body incidence angle; light beam duration; light beam size; and light beam focal distance. In an example, this device can comprise an array, grid, and/or matrix of light receivers which differ in: location and/or distance from a light emitter; and/or distance to body surface.

In an example, the frequency, color, and/or spectrum of a beam of light emitted from a light emitter can be changed over time to create a chronological sequence of beams of light with different frequencies, colors, and/or spectrums. In an example, the angle of a beam of light emitted from a light emitter can be changed over time to create a chronological sequence of beams of light with different projection and/or body incidence angles. In an example, the power or intensity of a beam of light emitted from a light emitter can be changed over time to create a chronological sequence of beams of light with different power or intensity levels. Such sequences can help to more accurately measure an analyte level in the body.

In an example, the frequency, color, and/or spectrum of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure an analyte level in the body. In an example, the projection angle of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure an analyte level in the body. In an example, the power and/or intensity of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure an analyte level in the body.

In an example, an emitter can separated from a receiver by a selected distance. In an example, there can be a selected distance between an emitter and a receiver. In an example, (an orthogonal component of) this distance can be measured along a circumferential axis. In an example, an emitter and a receiver can both be along the same circumferential line. In an example, (an orthogonal component of) this distance can be measured along a proximal-to-distal axis. In an example, an emitter and a receiver can both be along the same proximal-to-distal line. In an example, this selected distance can be expressed in inches and be within the range of 1/16″ to 2″. In an example, this selected distance can be expressed in metric units and be within the range of 2 mm to 5 cm. In an example, if this selected distance is along a circumferential axis, this distance can be expressed in (compass or polar coordinate) degrees and be within the range of 2 degrees to 60 degrees.

In an example, this device can have two (or more) emitters. In an example, two (or more) emitters can emit energy in a non-simultaneous (e.g. sequential) manner. In an example, a first emitter can be separated from a second emitter by a selected distance. In an example, there can be a selected distance between a first emitter and a second receiver. In an example, (an orthogonal component of) this distance can be measured along a circumferential axis. In an example, a first emitter and a second emitter can both be along the same circumferential line. In an example, (an orthogonal component of) this distance can be measured along a proximal-to-distal axis. In an example, a first emitter and a second emitter can both be along the same proximal-to-distal line. In an example, this selected distance can be expressed in inches and be within the range of 1/16″ to 2″. In an example, this selected distance can be expressed in metric units and be within the range of 2 mm to 5 cm. In an example, if this distance is along a circumferential axis, this selected distance can be expressed in (compass or polar coordinate) degrees and be within the range of 2 degrees to 60 degrees.

In an example, this device can have a circumferential array, matrix, or grid of four or more emitters, each of which is separated from the nearest other emitter by a distance within the range of 1/16″ to 2″. In an example, this device can have a circumferential array, matrix, or grid of four or more emitters, each of which is separated from the nearest other emitter by a distance within the range of 2 mm to 5 cm. In an example, this device can have a circumferential array, matrix, or grid of four or more emitters, each of which is separated from the nearest other emitter by a distance within the range of 2 degrees to 60 degrees. In an example, this device can have a circumferential array of emitters which spans between 25% and 100% of the cross-sectional perimeter circumference of a part of the body (e.g. wrist, arm, finger, ankle, or leg) to which the device is attached. In an example, this circumferential array of emitters can be even spaced or distributed, with the same pair-wise distance or number of degrees between adjacent emitters.

In an example, this device can have two (or more) receivers. In an example, a first receiver can be separated from a second receiver by a selected distance. In an example, there can be a selected distance between a first receiver and a second receiver. In an example, (an orthogonal component of) this distance can be measured along a circumferential axis. In an example, a first receiver and a second receiver can both be along the same circumferential line. In an example, (an orthogonal component of) this distance can be measured along a proximal-to-distal axis. In an example, a first receiver and a second receiver can both be along the same proximal-to-distal line. In an example, this selected distance can be expressed in inches and be within the range of 1/16″ to 2″. In an example, this selected distance can be expressed in metric units and be within the range of 2 mm to 5 cm. In an example, if this distance is along a circumferential axis, this selected distance can be expressed in (compass or polar coordinate) degrees and be within the range of 2 degrees to 60 degrees.

In an example, this device can have a circumferential array, matrix, or grid of four or more receivers, each of which is separated from the nearest other receiver by a distance within the range of 1/16″ to 2″. In an example, this device can have a circumferential array, matrix, or grid of four or more receivers, each of which is separated from the nearest other receiver by a distance within the range of 2 mm to 5 cm. In an example, this device can have a circumferential array, matrix, or grid of four or more receivers, each of which is separated from the nearest other receiver by a distance within the range of 2 degrees to 60 degrees. In an example, this device can have a circumferential array of receivers which spans between 25% and 100% of the cross-sectional perimeter circumference of a part of the body (e.g. wrist, arm, finger, ankle, or leg) to which the device is attached. In an example, this circumferential array of receivers can be even spaced or distributed, with the same pair-wise distance or number of degrees between adjacent receivers.

In an example, this device can comprise an array of emitters and receivers which is part of a wearable arcuate band or one or more segments (or housings) which are attached to a wearable arcuate band. In an example, this device can comprise a two-dimensional array of emitters and receivers which is part of a wearable arcuate band or one or more segments (or housings) which are attached to a wearable arcuate band. In an example, this device can comprise a three-dimensionally stacked array of emitters and receivers which is part of a wearable arcuate band or one or more segments (or housings) which are attached to a wearable arcuate band. In an example, data from this array can be analyzed to measure a person's analyte level.

In an example, an array of emitters and/or receivers can have a circumferential axis and a proximal-to-distal axis. In an example, this array can have at least three emitters and/or receivers along a circumferential axis and at least two emitters and/or receivers along a proximal-to-distal axis. In an example, an array can be formed from a plurality of sets of emitters and receivers, wherein each set forms the vertexes of a square or rectangle. In an example, an array can be formed from a plurality of sets of emitters and receivers, wherein each set forms the vertexes of a hexagon. In an example, an array can be formed from a plurality of sets of emitters and receivers, wherein each set forms a circle.

In an example, an array of emitters and receivers can have a square or rectangular shape. In an example, an array of emitters and receivers can have a hexagonal shape. In an example, an array of emitters and receivers can have a circular shape. In an example, an array of emitters and receivers can have a sunburst (e.g. radial spoke) shape. In an example, an array of emitters and receivers can have a cylindrical and/or ring shape. In an example, an array of emitters and receivers can have a conic section shape. In an example, an array of emitters and receivers can have a saddle shape. In an example, an array of emitters and receivers can have a helical shape.

In an example, a device can further comprise a track, channel, or slot along which an emitter, a receiver, or both can be moved. In an example, this movement can be done manually. In an example, this movement can be done automatically by one or more actuators. In an example, this track, channel, or slot can have a circumferential orientation. In an example, this track, channel, or slot can have a proximal-to-distal orientation. In an example, the distance between an emitter and a receiver can be adjusted by moving the emitter, the receiver, or both along such a track, channel, or slot. In an example, the location of an emitter and/or a receiver relative to a person's body can be adjusted by moving the emitter, the receiver, or both along such a track, channel, or slot. In an example, movement of an emitter, a receiver, or both along a track, channel, or slot can enable more accurate measurement of an analyte level in the body. In an example, movement of an emitter, a receiver, or both along a track, channel, or slot can enable customization of a device to the anatomy of a specific person for more accurate measurement of that person's analyte level.

In an example, a device can further comprise a rotating member which holds an emitter, a receiver, or both. In an example, rotation of this member can be done manually. In an example, this rotation can be done automatically by one or more actuators. In an example, the distance between an emitter and a receiver can be adjusted by rotating the rotating member. In an example, the location of an emitter and/or a receiver relative to a person's body can be adjusted by rotating the rotating member. In an example, movement of an emitter, a receiver, or both by a rotating member can enable more accurate measurement of an analyte level in the body. In an example, such movement of an emitter, a receiver, or both can enable customization of a device to the anatomy of a specific person for more accurate measurement of that person's analyte level.

In an example, this device can further comprise an energy source which powers an emitter, a receiver, a data processor, and/or a data transmitter. In an example, an energy source can be a battery. In an example, an energy source can transduce, harvest, and/or generate energy from body motion or kinetic energy. In an example, an energy source can transduce, harvest, and/or generate energy from ambient light energy. In an example, an energy source can transduce, harvest, and/or generate energy from body thermal energy. In an example, an energy source can transduce, harvest, and/or generate energy from ambient electromagnetic energy.

In an example, a wearable device can further comprise a data processor which analyzes the spectrum of light received by a light receiver in order to measure the amount of an analyte in body tissue. In an example, data concerning light received by a light receiver can be transmitted to a remote data processor by the wireless data transmitter and analysis of this data can occur in that remote data processor. In an example, an implanted cardiac rhythm management can further comprise a data processor. In an example, data concerning light received by a light receiver can be transmitted to the wireless data receiver and analysis of this data can occur in the data processor within the implanted cardiac rhythm management device.

In an example, this device can further comprise a wireless data transmitter and/or data receiver. In various examples, this device can be in wireless communication with an external device selected from the group consisting of: a cell phone, an electronic tablet, electronically-functional eyewear, a home electronics portal, an implanted medical device, an internet portal, a laptop computer, a mobile computer, a mobile phone, a remote computer, a remote control unit, a smart phone, a smart utensil, a television set, and a wearable data processing hub. In an example, additional data processing and analysis can be done within an external device.

In an example, this device can further comprise an energy barrier between an emitter and a receiver which reduces the transmission of energy from the emitter to the receiver. In an example, an energy barrier between a light emitter and a light receiver can be opaque. In an example, an energy barrier between a light emitter and a light receiver can be compressible, flexible, and/or elastic. In an example, an energy barrier can comprise compressible foam. In an example, an energy barrier can be an inflatable member (such as a balloon) which is filled with a gas or liquid. In an example, an energy barrier can have a linear shape. In an example, an energy barrier can have a circular, elliptical, sinusoidal, or other arcuate shape. In an example, an energy barrier can surround a receiver. In an example, an energy barrier can surround an emitter.

In an example, this device can further comprise an energy conductor between an emitter and a receiver which increases the transmission of energy from the emitter to the receiver. In an example, an energy conductor between a light emitter and a light receiver can be an optical lens and/or fiber optic conduit.

In an example, this device can further comprise one or more other types of biometric or environmental sensors in addition to the primary emitters and receivers discussed above. In an example, the primary emitter and the primary receiver of this device, discussed above, can be a light emitter and a light receiver, but the device can also include a (non-light-spectrum) electromagnetic emitter and a (non-light-spectrum) electromagnetic receiver. In an example, the primary emitter and the primary receiver of this device, discussed above, can be a (non-light-spectrum) electromagnetic emitter and a (non-light-spectrum) electromagnetic receiver, but the device can also include a light emitter and a light receiver. In an example, this device can comprise both light energy and electromagnetic energy sensors for measuring an analyte level in the body. In an example, this device can comprise both spectroscopic and microwave energy sensors for measuring an analyte level in the body.

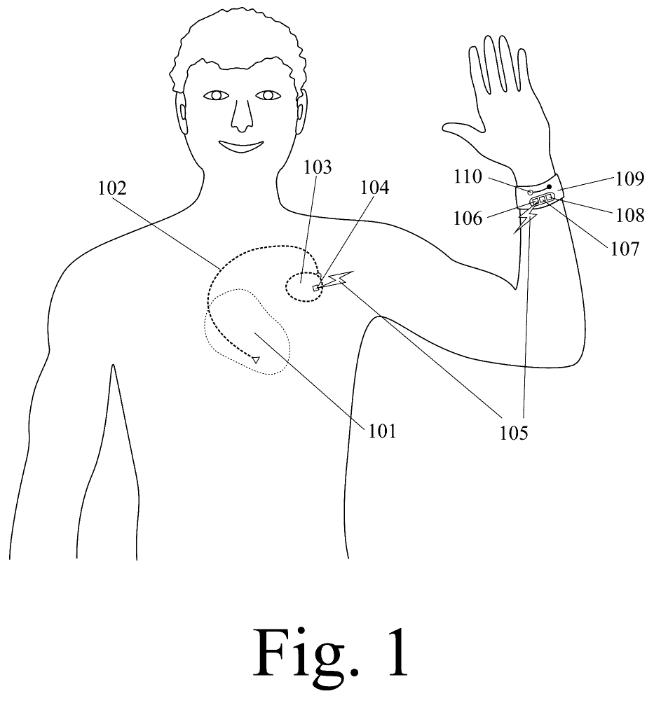

This concludes the introductory section and begins discussion of specific figures. FIG. 1 shows an example of how this invention can be embodied in a system (or device) for automatic adjustment of an implanted cardiac management device comprising: (a) a wearable component which is configured to be worn on a person's body or clothing; (b) a biometric sensor which is configured to be held in proximity to the surface of the person's body by the wearable component; (c) a data processor which receives data from the biometric sensor; and (d) an implanted cardiac management device which is configured to manage (or control or change) the person's cardiac function, wherein the operation of the implanted cardiac management device is automatically adjusted based on analysis of data from the biometric sensor. In this example, being in proximity to the surface of the person's body can be defined as having at least one part which is worn less than three inches away from the person's body.

Specifically, the system shown in FIG. 1 comprises: (a) wearable component 109 which is configured to be worn on a person's body or clothing; (b) biometric sensor 110 which is configured to be held in proximity to the surface of the person's body by the wearable component; (c) data processor 107 which receives data from the biometric sensor; and (d) an implanted cardiac management device which is configured to manage (or control or change) the person's cardiac function, wherein the operation of the implanted cardiac management device is automatically adjusted based on analysis of data from the biometric sensor, wherein this implanted cardiac management device further comprises electronics housing 103, wire lead 102 which is configured to provide electromagnetic communication between the electronics housing and the person's heart 101, and data receiver (and transmitter) 104 which receives wirelessly-transmitted data 105. The system in this example further comprises power source 108 and data transmitter (and receiver) 106.

In the example shown in FIG. 1, the wearable component of the system is worn on a person's wrist like a wrist band or smart watch. In various examples, the wearable component of this system can be selected from the group consisting of an armlet, bangle, bracelet, cuff, fitness band, gauntlet, sleeve, smart watch, strap, watch, and wrist band.

In an example, the biometric sensor of this system can be a light sensor which receives light energy which has been reflected from, or passed through, body tissue, organs, and/or fluid. In an example, this light sensor can be a spectroscopic sensor. A spectroscopic sensor can collect data concerning the spectrum of light energy which has been reflected from (or has passed through) body tissue, organs, and/or fluid. This data concerning light energy is used to analyze the spectral distribution of that light and thereby infer the chemical composition and/or physical configuration of the body tissue, organs, and/or fluid. In an example, the operation of the implanted cardiac management device can be adjusted based on (changes in) biological parameters or physiological conditions which are detected by the spectroscopic sensor.

In an example, the biometric sensor of this system can be an electromagnetic energy sensor. In an example, an electromagnetic energy sensor can be an electromagnetic energy receiver which receives electromagnetic energy which is naturally generated by the electromagnetic activity of body tissue and/or organs. In an example, an electromagnetic energy sensor can comprise an electromagnetic energy emitter at a first location relative to body tissue and an electromagnetic energy receiver at a second location relative to body tissue, wherein the electromagnetic energy receiver receives energy which has been transmitted from the electromagnetic energy emitter through body tissue. In an example, an electromagnetic energy receiver can collect data concerning (changes in) the conductivity, resistance, and/or impedance of electromagnetic energy transmitted through body tissue from the electromagnetic energy emitter to the electromagnetic energy receiver. In an example, an electromagnetic energy emitter and an electromagnetic energy receiver can together be referred to as an electromagnetic energy sensor. In an example, the operation of the implanted cardiac management device can be adjusted based on (changes in) biological parameters or physiological conditions which are detected by the electromagnetic energy sensor.

In an example, an electromagnetic energy sensor can be an electromagnetic muscle activity sensor. In an example, an electromagnetic energy sensor can be an electromyographic (EMG) sensor. In an example, an electromagnetic muscle activity sensor can collect data concerning the emission of electromagnetic energy by a person's muscles and/or the nerves which innervate those muscles. In an example, an electromagnetic energy sensor can collect data concerning changes in transmission of electromagnetic energy from an emitter to a receiver due to changes in electromagnetic muscle activity. In an example, the operation of the implanted cardiac management device can be adjusted based on (changes in) biological parameters or physiological conditions which are detected by an EMG sensor.

In an example, the implanted cardiac management device of this system can be an implanted pacemaker. In this example, an electronics housing of a cardiac management device is configured to be in electromagnetic communication with a person's heart via a wire lead. In another example, an electronics housing of a cardiac management device can be configured to be in direct contact with a person's heart. In various examples, cardiac functioning parameters which can be adjusted based on data from a wearable biometric sensor can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

Relevant example variations which are discussed in prior portions of this disclosure can also be applied to the example shown here in FIG. 1. These variations are not all repeated here in order to avoid redundancy among the descriptions accompanying each of the specific figures. Example variations discussed previously include, but are not limited to, different types and locations of wearable components, different types of biometric sensors, biometric sensor arrays with sensors with different operating parameters, examples of specific biometric parameters and physiological conditions to be monitored, and examples of specific responses by the implanted cardiac management device in response to specific biometric parameters and physiological conditions.

FIG. 2 shows another example of how this invention can be embodied in a system (or device) for automatic adjustment of an implanted cardiac management device comprising: (a) a wearable component which is configured to be worn on a person's body or clothing; (b) a biometric sensor which is configured to be held in proximity to the surface of the person's body by the wearable component; (c) a data processor which receives data from the biometric sensor; and (d) an implanted cardiac management device which is configured to manage (or control or change) the person's cardiac function, wherein the operation of the implanted cardiac management device is automatically adjusted based on analysis of data from the biometric sensor. In this example, being in proximity to the surface of the person's body can be defined as having at least one part which is worn less than three inches away from the person's body.

Specifically, the system shown in FIG. 2 comprises: (a) wearable component 209 which is configured to be worn on a person's body or clothing; (b) biometric sensor 210 which is configured to be held in proximity to the surface of the person's body by the wearable component; (c) data processor 207 which receives data from the biometric sensor; and (d) an implanted cardiac management device which is configured to manage (or control or change) the person's cardiac function, wherein the operation of the implanted cardiac management device is automatically adjusted based on analysis of data from the biometric sensor, wherein this implanted cardiac management device further comprises electronics housing 203, wire lead 202 which is configured to provide electromagnetic communication between the electronics housing and the person's heart 201, and data receiver (and transmitter) 204 which receives wirelessly-transmitted data 205. The system in this example further comprises power source 208 and data transmitter (and receiver) 206.

In the example shown in FIG. 2, the wearable component of the system is worn on a person's finger like a ring. In various examples, the wearable component of this system can be selected from the group consisting of a finger ring, finger sleeve, artificial finger nail, finger nail attachment, finger tip (thimble), and glove.

In an example, the biometric sensor of this system can be a light sensor which receives light energy which has been reflected from, or passed through, body tissue, organs, and/or fluid. In an example, this light sensor can be a spectroscopic sensor. A spectroscopic sensor can collect data concerning the spectrum of light energy which has been reflected from (or has passed through) body tissue, organs, and/or fluid. This data concerning light energy is used to analyze the spectral distribution of that light and thereby infer the chemical composition and/or physical configuration of the body tissue, organs, and/or fluid. In an example, the operation of the implanted cardiac management device can be adjusted based on (changes in) biological parameters or physiological conditions which are detected by the spectroscopic sensor.

In an example, the biometric sensor of this system can be an electromagnetic energy sensor. In an example, an electromagnetic energy sensor can be an electromagnetic energy receiver which receives electromagnetic energy which is naturally generated by the electromagnetic activity of body tissue and/or organs. In an example, an electromagnetic energy sensor can comprise an electromagnetic energy emitter at a first location relative to body tissue and an electromagnetic energy receiver at a second location relative to body tissue, wherein the electromagnetic energy receiver receives energy which has been transmitted from the electromagnetic energy emitter through body tissue. In an example, an electromagnetic energy receiver can collect data concerning (changes in) the conductivity, resistance, and/or impedance of electromagnetic energy transmitted through body tissue from the electromagnetic energy emitter to the electromagnetic energy receiver. In an example, an electromagnetic energy emitter and an electromagnetic energy receiver can together be referred to as an electromagnetic energy sensor. In an example, the operation of the implanted cardiac management device can be adjusted based on (changes in) biological parameters or physiological conditions which are detected by the electromagnetic energy sensor.

In an example, the implanted cardiac management device of this system can be an implanted pacemaker. In this example, an electronics housing of a cardiac management device is configured to be in electromagnetic communication with a person's heart via a wire lead. In another example, an electronics housing of a cardiac management device can be configured to be in direct contact with a person's heart. In various examples, cardiac functioning parameters which can be adjusted based on data from a wearable biometric sensor can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

Relevant example variations which are discussed in prior portions of this disclosure can also be applied to the example shown here in FIG. 2. These variations are not all repeated here in order to avoid redundancy among the descriptions accompanying each of the specific figures. Example variations discussed previously include, but are not limited to, different types and locations of wearable components, different types of biometric sensors, biometric sensor arrays with sensors with different operating parameters, examples of specific biometric parameters and physiological conditions to be monitored, and examples of specific responses by the implanted cardiac management device in response to specific biometric parameters and physiological conditions.

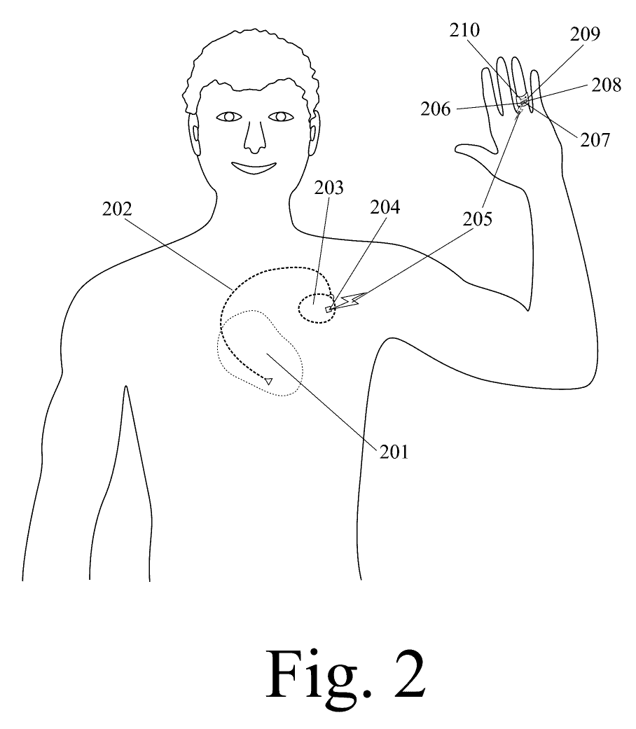

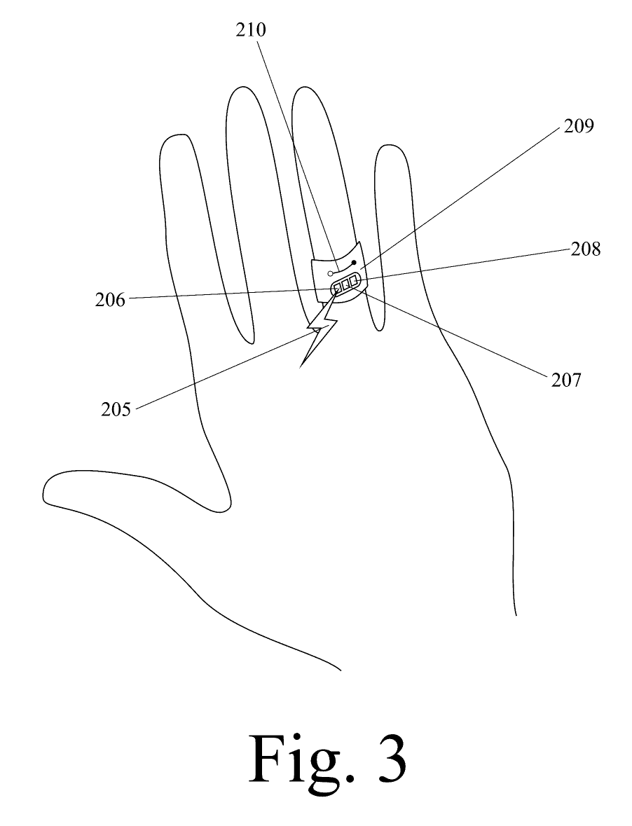

FIG. 3 shows a close-up view of the wearable component of the system that was shown in FIG. 2 in order to provide a clearer view of the components of the wearable component of the system.

FIG. 4 shows another example of how this invention can be embodied in a system (or device) for automatic adjustment of an implanted cardiac management device comprising: (a) a wearable component which is configured to be worn on a person's body or clothing; (b) a biometric sensor which is configured to be held in proximity to the surface of the person's body by the wearable component; (c) a data processor which receives data from the biometric sensor; and (d) an implanted cardiac management device which is configured to manage (or control or change) the person's cardiac function, wherein the operation of the implanted cardiac management device is automatically adjusted based on analysis of data from the biometric sensor. In this example, being in proximity to the surface of the person's body can be defined as having at least one part which is worn less than three inches away from the person's body.

Specifically, the system shown in FIG. 4 comprises: (a) wearable component 409 which is configured to be worn on a person's body or clothing; (b) biometric sensor 410 which is configured to be held in proximity to the surface of the person's body by the wearable component; (c) data processor 407 which receives data from the biometric sensor; and (d) an implanted cardiac management device which is configured to manage (or control or change) the person's cardiac function, wherein the operation of the implanted cardiac management device is automatically adjusted based on analysis of data from the biometric sensor, wherein this implanted cardiac management device further comprises electronics housing 403, wire lead 402 which is configured to provide electromagnetic communication between the electronics housing and the person's heart 401, and data receiver (and transmitter) 404 which receives wirelessly-transmitted data 405. The system in this example further comprises power source 408 and data transmitter (and receiver) 406.

In the example shown in FIG. 4, the wearable component of the system is worn on a person's ear. In an example, the wearable component of this system can be configured to be worn on, around, or within a person's ear. In an example, the wearable component can be inserted (partially or fully) into the ear canal, attached to the earlobe, worn around a portion of the outer ear, or a combination thereof. In an example, an ear-worn wearable component of this system can also include a prong, arm, or other protrusion which extends forward onto the person's temple and/or their forehead. In an example, the wearable component of this system can be a “hearable” device. In an example, the wearable component of this system can be selected from the group consisting of: ear bud, ear hook, ear plug, ear ring, earlobe clip, earphone, earpiece, earring, ear-worn Bluetooth communication device, electroencephalographic (EEG) sensor, oximeter, headphone, headset, and hearing aid.

In an example, the biometric sensor of this system can be a light sensor which receives light energy which has been reflected from, or passed through, body tissue, organs, and/or fluid. In an example, this light sensor can be a spectroscopic sensor. A spectroscopic sensor can collect data concerning the spectrum of light energy which has been reflected from (or has passed through) body tissue, organs, and/or fluid. This data concerning light energy is used to analyze the spectral distribution of that light and thereby infer the chemical composition and/or physical configuration of the body tissue, organs, and/or fluid. In an example, the operation of the implanted cardiac management device can be adjusted based on (changes in) biological parameters or physiological conditions which are detected by the spectroscopic sensor.

In an example, the biometric sensor of this system can be an electromagnetic energy sensor. In an example, an electromagnetic energy sensor can be an electromagnetic energy receiver which receives electromagnetic energy which is naturally generated by the electromagnetic activity of body tissue and/or organs. In an example, an electromagnetic energy sensor can comprise an electromagnetic energy emitter at a first location relative to body tissue and an electromagnetic energy receiver at a second location relative to body tissue, wherein the electromagnetic energy receiver receives energy which has been transmitted from the electromagnetic energy emitter through body tissue. In an example, an electromagnetic energy receiver can collect data concerning (changes in) the conductivity, resistance, and/or impedance of electromagnetic energy transmitted through body tissue from the electromagnetic energy emitter to the electromagnetic energy receiver. In an example, an electromagnetic energy emitter and an electromagnetic energy receiver can together be referred to as an electromagnetic energy sensor. In an example, the operation of the implanted cardiac management device can be adjusted based on (changes in) biological parameters or physiological conditions which are detected by the electromagnetic energy sensor.

In an example, an electromagnetic energy sensor can be an electromagnetic brain activity sensor. In an example, an electromagnetic energy sensor can be an electroencephalographic (EEG) sensor. In an example, an electromagnetic energy sensor can be a wearable electromagnetic brain activity sensor and/or wearable electroencephalographic (EEG) sensor. In an example, an electromagnetic energy sensor can be a brain activity sensor which collects data concerning the natural emission of electromagnetic energy by a person's brain. In an example, an electromagnetic energy sensor can comprise an electromagnetic energy emitter and an electromagnetic energy receiver which are in proximity to a person's head. In an example, an electromagnetic energy sensor can collect data concerning changes in transmission of electromagnetic energy from the emitter to the receiver due to changes in electromagnetic brain activity. In an example, an electromagnetic brain activity sensor can measure voltage fluctuations resulting from ionic current within the neurons of the brain. In an example, the operation of the implanted cardiac management device can be adjusted based on (changes in) biological parameters or physiological conditions which are detected by an EEG sensor.

In an example, the implanted cardiac management device of this system can be an implanted pacemaker. In this example, an electronics housing of a cardiac management device is configured to be in electromagnetic communication with a person's heart via a wire lead. In another example, an electronics housing of a cardiac management device can be configured to be in direct contact with a person's heart. In various examples, cardiac functioning parameters which can be adjusted based on data from a wearable biometric sensor can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

Relevant example variations which are discussed in prior portions of this disclosure can also be applied to the example shown here in FIG. 4. These variations are not all repeated here in order to avoid redundancy among the descriptions accompanying each of the specific figures. Example variations discussed previously include, but are not limited to, different types and locations of wearable components, different types of biometric sensors, biometric sensor arrays with sensors with different operating parameters, examples of specific biometric parameters and physiological conditions to be monitored, and examples of specific responses by the implanted cardiac management device in response to specific biometric parameters and physiological conditions.

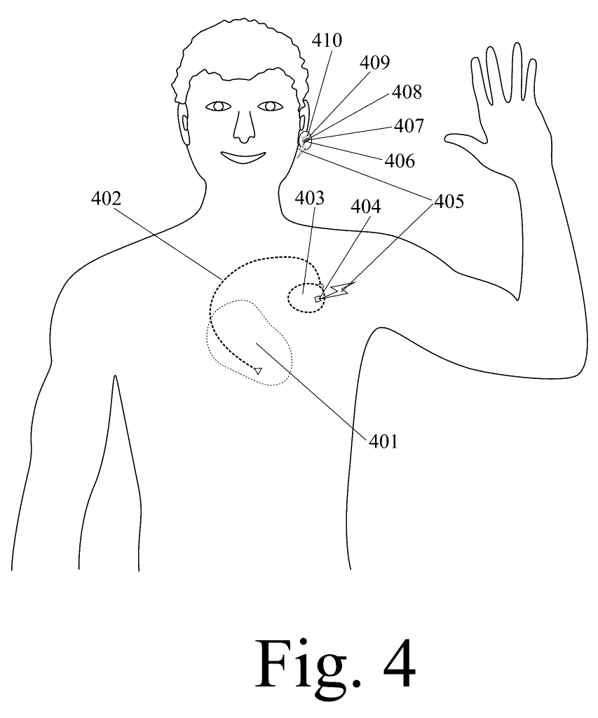

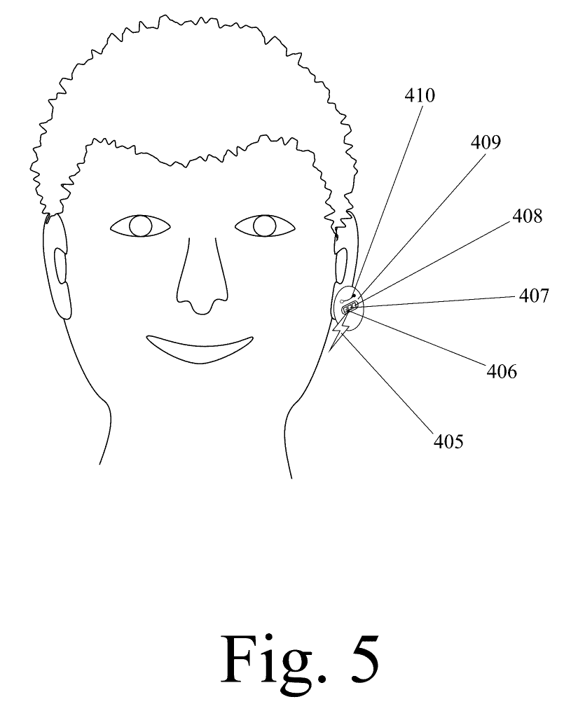

FIG. 5 shows a close-up view of the wearable component of the system that was shown in FIG. 4 in order to provide a clearer view of the components of the wearable component of the system.

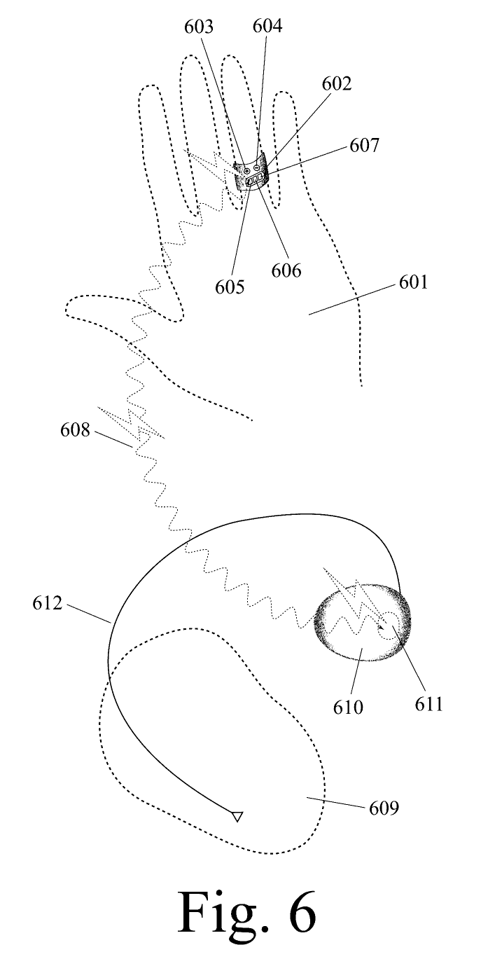

FIG. 6 shows an example of how this invention can be embodied in an integrated system for managing cardiac rhythm including both a wearable device and an implanted device, wherein this system comprises: (a) a wearable device which is configured to be worn by a person, wherein the wearable device further comprises a light emitter which is configured to emit light toward the person's body tissue, a light receiver which is configured to receive light from the light emitter after the light has passed through and/or been reflected from the person's body tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's body tissue; and (d) wherein the operation of the cardiac rhythm management device is changed based on the amount of the analyte in the person's body tissue.

In FIG. 6, the wearable component of the system is a finger ring. In this example, the analyte is oxygen level. In this example, this invention is embodied in an integrated system for managing cardiac rhythm including both a wearable device and an implanted device, wherein this system comprises: (a) a finger ring which is configured to be worn by a person, wherein the finger ring further comprises a light emitter which is configured to emit light toward the person's finger tissue, a light receiver which is configured to receive light from the light emitter after the light has passed through and/or been reflected from the person's finger tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the oxygen level in the person's finger tissue; and (d) wherein the operation of the cardiac rhythm management device is changed based on oxygen level in the person's finger tissue.

The upper portion of FIG. 6 shows a person's hand 6001 with finger ring 6002 being worn on a finger. Finger ring 6002 further comprises light emitter 6003 which is configured to emit light toward the person's finger tissue and light receiver 6004 which is configured to receive light from the light emitter after the light has passed through and/or been reflected from the person's finger tissue. Finger ring 6002 further comprises wireless data transmitter 6005, data processor 6006, and power source (or transducer) 6007.

The lower portion of FIG. 6 shows a person's heart 6009 and an implanted cardiac rhythm management device 6010 which is in electromagnetic communication the heart via wire lead 6012. In an example, an implanted cardiac rhythm management device can be a pacemaker or defibrillator. In an example, an implanted cardiac rhythm management device can be implanted directly in the heard and not require a wire lead to be in electromagnetic communication with the heart. Cardiac rhythm management device 6010 further comprises a wireless data receiver 6011 which receives data 6008 transmitted wirelessly from data transmitter 6005. In this example, wireless data transmitter 6005 and wireless data receiver 6011 are in direct electromagnetic communication with each other. In another example, wireless data transmitter 6005 can transmit data to a remote device (which can process data) and the remote device, in turn, can transmit data to wireless data receiver 6011.

Together, system components in the upper and lower portions of FIG. 6 comprise an integrated system for managing cardiac rhythm including both a wearable device and an implanted device. The synergistic integration of the wearable device and implanted device can enable cardiac rhythm management that is superior to that provided by either component alone. For example, without an implanted cardiac rhythm management device, a wearable device alone can provide information on oxygenation levels in body extremities, but does not provide automatic therapeutic correction for oxygenation deficiency in body extremities. Also, without a wearable device component to measure body oxygen levels in body extremities, an implanted cardiac rhythm management device alone is not aware of oxygen deficiencies in body extremities. Working together in an integrated system, a wearable device for measure body oxygen level in body extremities and an implanted device for cardiac rhythm management can help to prevent oxygen deficiencies in body extremities. This can help to avoid physiological dysfunction and potentially even limb loss due to poor circulation and oxygenation.

In an example, the spectral distribution of light emitted from the light emitter and the spectral distribution of light received by the light receiver are analyzed in order to measure the oxygen level in the person's finger tissue. In an example, body tissue can be understood to include blood, interstitial fluid, and other body fluids. In an example, the operation of the cardiac rhythm management device is changed based on oxygen level in the person's finger tissue. In an example, this system can change the frequency and/or magnitude of electromagnetic pulses delivered to a person's heart when analysis of data from the light receiver indicates a change in oxygen level in body tissue. In an example, the system can increase the frequency and/or magnitude of electromagnetic pulses delivered to the person's heart when analysis of data from the light receiver indicates a low oxygen level in body tissue. In an example, the system can decrease the frequency and/or magnitude of electromagnetic pulses delivered to the person's heart when analysis of data from the light receiver indicates a high oxygen level in body tissue.

In an example, a finger ring can have a circular cross-sectional shape. In an example, a finger ring can have a circular circumference. In an example, a finger ring can have an inward side which is configured to face toward the surface of a person's finger and an outward side which is configured to face away from the surface of a person's finger. In an example, a light emitter can emit light from the inward side of a wearable device toward the surface of a person's body (e.g. finger, wrist, arm, ear, or leg). In an example, a light receiver can receive light into the inward side of a wearable device which has passed through and/or been reflected from a person's body tissue. In an example, there can be a flexible and/or compressible light barrier between a light emitter and a light receiver. In an example, a light emitter and a light receiver can be on the same circumferential line (e.g. circle) of a wearable device, but at different radial locations around this circumference. In an example, a light emitter and a light receiver can be on the same radial location around a wearable device, but on different circumferential lines (e.g. circles). In an example, there can be two or more light emitters and one light receiver on a wearable device. In an example, there is one light emitter and two or more light receivers on a wearable device. In an example, this system can comprise an array of light emitters and light receivers as discussed elsewhere in this disclosure or priority-linked disclosures.

In an example, a light emitter can emit coherent light. In an example, a light emitter can be a laser. In an example, a light emitter can be a Light Emitting Diode (LED). In an example, a light emitter can emit infrared or near-infrared light. In an example, a light emitter can emit ultraviolet light. In an example, a light emitter emit red light and/or be a red-light laser. In an example, a light emitter emit green light and/or be a green-light laser. In an example, a light emitter can emit white light and/or be a white-light laser. In an example, a wearable device can include can be two or more light emitters. In an example, a wearable device can include a red light emitter and a green light emitter. In an example, a light emitter can emit light with frequency and/or spectrum changes over time. In an example, a light emitter can emit a sequence of light pulses at different selected frequencies. In an example, a light emitter can emit polarized light. In an example, the polarization of light can change after the light passes through and/or is reflected from body tissue and these changes can be used to measure an analyte level in the body.

In an example, a light emitter and a light receiver together can comprise a spectroscopic (or “spectroscopy”) sensor. In an example, the spectrum of light energy is changed when the light energy passes through body tissue and/or is reflected from body tissue. In an example, changes in the spectrum of light energy which has passed through and/or been reflected from body tissue can be analyzed to detect the composition and/or configuration of body tissue. In an example, these changes in the spectrum of light energy can be analyzed to provide information on the composition of body tissue which, in turn, enables measurement of an analyte level in the body. In an example, a light emitter and a light receiver together can comprise a sensor selected from the group consisting of: backscattering spectrometry sensor, infrared spectroscopy sensor, ion mobility spectroscopic sensor, mass spectrometry sensor, Near Infrared Spectroscopy sensor (NIS), Raman spectroscopy sensor, spectrometry sensor, spectrophotometer, spectroscopy sensor, ultraviolet spectroscopy sensor, and white light spectroscopy sensor.

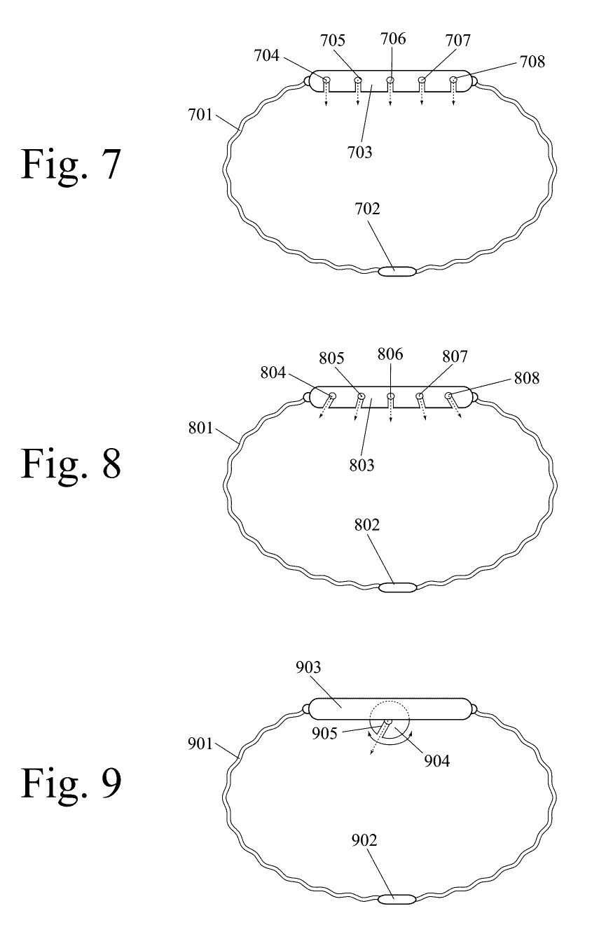

FIG. 7 shows an example of a wearable device for the arm with a plurality of close-fitting biometric sensors. This figure shows the device from a side perspective, as it would appear encircling a lateral cross-section of a person's wrist (or other portion of the person's arm). Described generally, the example shown in FIG. 7 is an arcuate wrist-worn device with a circumferentially-distributed array of biometric sensors. A series of circumference-center-facing biometric sensors are distributed along different locations on a portion of the circumference of the device. In this example, the array of sensors is distributed along the circumference-center-facing surface of an enclosure which is on the anterior (upper) portion of the device. In another example, an array of sensors can be distributed along the circumference-center-facing surface of a band or strap.

Having a circumferentially-distributed array of sensors allows a wearable device to record biometric measurements from different locations along the circumference of a person's wrist. This can help to find the best location on a person's wrist from which to most-accurately record biometric measurements. Having a circumferentially-distributed array of sensors can also enable a device to record biometric measurements from substantially the same location on a person's wrist, even if the device is unintentionally slid, shifted, and/or partially-rotated around the person's wrist. A different primary sensor can selected to record data when the device slides, shifts, and/or rotates. This can help to reduce biometric measurement errors when the device is slid, shifted, and/or partially-rotated around a person's wrist.

More specifically, the example shown in FIG. 7 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; (c) a first biometric sensor at a first location in the enclosure which is configured to record biometric data concerning the person's arm tissue; and (d) a second biometric sensor at a second location in the enclosure which is configured to record biometric data concerning the person's arm tissue, wherein the distance along the circumference of the device from the first location to second location is at least a quarter inch.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, a plurality of sensors can be housed within a single enclosure. In another example, different sensors can be housed in different enclosures. In another example, sensors can be located along the circumference-center-facing surface of an attachment member. In an example, there can be a display screen on the outward-facing surface of an enclosure.

In an example, first and second biometric sensors can be spectroscopic sensors which are each configured to measure the spectrum of light energy reflected from (and/or absorbed by) tissue of the person's arm. In an example, first and second biometric sensors can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 7 includes: strap (or band) 701, strap (or band) connector 702, enclosure 703, and spectroscopic sensors 704, 705, 706, 707, and 708. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 8 shows another example of a wearable device for the arm with a plurality of close-fitting biometric sensors. This figure shows the device from a side perspective, as it would appear encircling a lateral cross-section of a person's wrist (or other portion of the person's arm).

The example shown in FIG. 8 is like the one shown in FIG. 7 except that different sensors in the array of sensors direct light energy onto the surface of an arm at different angles relative to an enclosure. Having an array of sensors which direct light energy onto the surface of the arm at different angles relative to an enclosure can enable a device to record biometric measurements with substantially the same angle of incidence, even if the enclosure is tilted with respect to the surface of the person's wrist. A different primary sensor with a different angle of light projection can be selected to record data when the enclosure is tilted. For example, when an enclosure is parallel to the surface of the person's wrist, then a sensor with a 90 degree light projection angle (relative to the enclosure) can be selected so that light is projected onto the surface of the arm in a perpendicular manner. However, when the enclosure is tilted at a 20 degree angle relative to the surface of the person's wrist, then a sensor with a 70 degree angle (relative to the enclosure) can be selected so that light is again projected onto the surface of the arm in a perpendicular manner.

The example shown in FIG. 8 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; (c) a first spectroscopic sensor in the enclosure which is configured to project a beam of light onto the arm surface at a first angle relative to the enclosure; and (d) a second spectroscopic sensor in the enclosure which is configured to project a beam of light onto the arm surface at a second angle relative to the enclosure, wherein the first angle differs from the second angle by at least 10 degrees.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, a plurality of sensors can be housed within a single enclosure. In another example, different sensors can be housed in different enclosures. In another example, sensors can be located along the circumference-center-facing surface of an attachment member. In an example, there can be a display screen on the outward-facing surface of an enclosure.

With respect to specific components, the example shown in FIG. 8 includes: strap (or band) 801, strap (or band) connector 802, enclosure 803, and spectroscopic sensors 804, 805, 806, 807, and 808. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 9 shows an example of a wearable device for the arm with a close-fitting biometric sensor. This figure shows the device from a side perspective, as it would appear encircling a lateral cross-section of a person's wrist (or other portion of the person's arm).

Described generally, the example shown in FIG. 9 is an arcuate wrist-worn device with a rotating light-projecting spectroscopic sensor, wherein rotation of this sensor changes the angle at which it projects light onto the surface of a person's arm. In this example, the rotating light-projecting spectroscopic sensor is on the circumference-center-facing surface of an enclosure which is on the anterior (upper) portion of the device. In another example, such a sensor can be on the circumference-center-facing surface of a band or strap.

Having a rotating light-projecting spectroscopic sensor can enable a device to record biometric measurements with substantially the same angle of incidence, even if an enclosure is tilted with respect to the surface of the person's wrist. For example, when the enclosure is parallel to the surface of the person's wrist, then the rotating sensor is automatically rotated to project light at a 90 degree angle (relative to the enclosure) so that light is projected onto the surface of the arm in a perpendicular manner. However, when the enclosure is tilted at a 20 degree angle relative to the surface of the person's wrist, then the rotating sensor is automatically rotated to project light at a 70 degree angle (relative to the enclosure) so that light is again projected onto the surface of the arm in a perpendicular manner.

The example shown in FIG. 9 is a wearable device for the arm with a close-fitting biometric sensor comprising: (a) an attachment member, such as a strap or band, which is configured to span at least a portion of the circumference of a person's arm; (b) an enclosure which is part of (or attached to) the attachment member; and (c) a rotating light-projecting spectroscopic sensor, wherein this sensor can be rotated relative to the enclosure and wherein rotation of this sensor relative to the enclosure changes the angle at which the sensor projects light onto the surface of a person's arm.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, there can be a display screen on the outward-facing surface of an enclosure.

With respect to specific components, the example shown in FIG. 9 includes: strap (or band) 901, strap (or band) connector 902, enclosure 903, rotating member 904, and light-projecting spectroscopic sensor 905. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

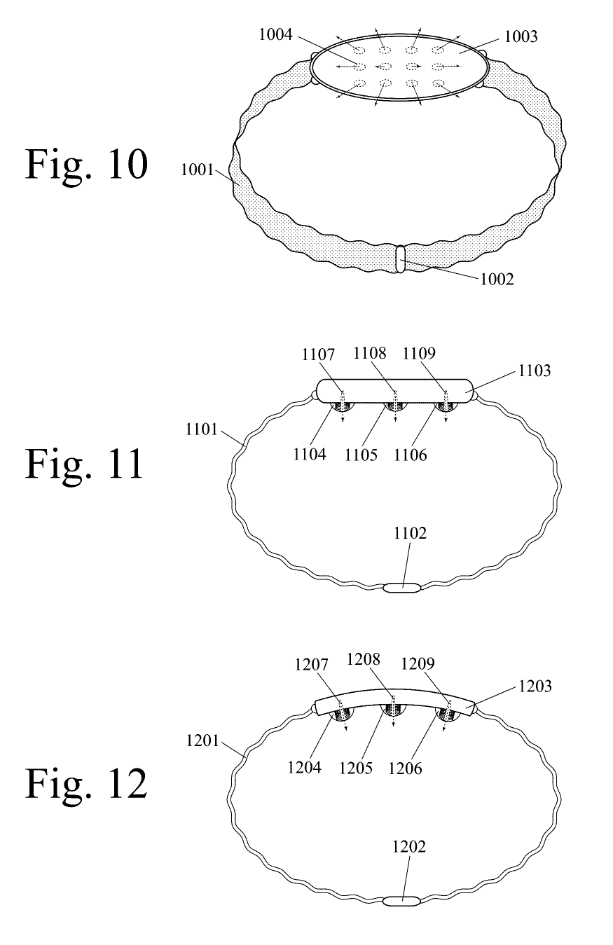

FIG. 10 shows another example of a wearable device for the arm with a plurality of close-fitting biometric sensors. This figure shows the device from a non-perpendicular lateral perspective, as it would appear encircling a lateral cross-section of a person's wrist (or other portion of the person's arm).

Described generally, the example shown in FIG. 10 is an arcuate wrist-worn device with a two-dimensional array of spectroscopic sensors. Sensors in this two-dimensional array differ in location circumferentially (they are at different locations around the circumference of the device) and laterally (they are at different locations along axes which are perpendicular to the circumference of the device). In this example, the two-dimensional sensor array is part of the circumference-center-facing surface of an enclosure which is on the anterior (upper) portion of the device. In another example, a two-dimensional sensor array can be on the circumference-center-facing surface of a band or strap.

Having a two-dimensional sensor array allows a wearable device to record biometric measurements from multiple locations on a person's wrist. This can help to find the best location on a person's wrist from which to most-accurately record biometric measurements. Having a two-dimensional sensor array can also enable a device to record biometric measurements from substantially the same location on a person's wrist even if the device is rotated around the person's wrist or slid up or down the person's arm. A different primary sensor can be automatically selected to record data when the device rotates or slides.

More specifically, the example shown in FIG. 10 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; and (c) a two-dimensional sensor array which is part of the enclosure, wherein sensors in this two-dimensional array differ in location along a portion of the circumference of the device, and wherein sensors in this two-dimensional array differ in location along axes which are perpendicular to the circumference of the device.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, there can be a display screen on the outward-facing surface of an enclosure.

In an example, sensors in a two-dimensional sensor array can be spectroscopic sensors which are each configured to measure the spectrum of light energy reflected from (and/or absorbed by) tissue of the person's arm. In an example, sensors in a two-dimensional sensor array can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 10 includes: a strap (or band) 1001, a strap (or band) connector 1002, an enclosure 1003, and a two-dimensional spectroscopic sensor array which includes sensor 1004. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 11 shows another example of a wearable device for the arm with a plurality of close-fitting biometric sensors. This figure shows the device from a side perspective, as it would appear encircling a lateral cross-section of a person's wrist (or other portion of the person's arm).

Described generally, the example shown in FIG. 11 is an arcuate wrist-worn device with a plurality of spectroscopic sensors, wherein each of these sensors is pushed toward the surface of an arm in order to stay in close contact with the surface of the arm even if the enclosure is shifted or tilted away from the surface of the arm. In this example, the spectroscopic sensors are on the circumference-center-facing portion of an enclosure. In this example, each of the spectroscopic sensors is pushed toward the surface of the arm by a spring mechanism. In another example, each of the spectroscopic sensors can be pushed toward the surface by a hydraulic mechanism, a pneumatic mechanism, or a microscale electromagnetic actuator.

More specifically, the example shown in FIG. 11 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; and (c) a plurality of sensors which are part of the enclosure, wherein each sensor in this plurality of sensors is configured to be pushed toward the surface of the arm by a spring mechanism in order to keep the sensor in close contact with the surface of the arm.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, sensors of this device can be spectroscopic sensors which are each configured to measure the spectrum of light energy reflected from (and/or absorbed by) tissue of the person's arm. In an example, sensors of this device can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 11 includes: a strap (or band) 1101; a strap (or band) connector 1102; an enclosure 1103; a plurality of spectroscopic sensors (1107, 1108, and 1109); and a plurality of spring mechanisms (1104, 1105, and 1106) which are configured to push the sensors inward toward the center of the device. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 12 shows another example of a wearable device for the arm with a plurality of close-fitting biometric sensors. This figure shows the device from a side perspective, as it would appear encircling a lateral cross-section of a person's wrist (or other portion of the person's arm). The example shown in FIG. 12 is similar to the one shown in FIG. 11, except that the enclosure housing biometric sensors in FIG. 12 has a curved circumference-center-facing surface rather than a flat circumference-center-facing surface.

With respect to specific components, the example shown in FIG. 12 includes: a strap (or band) 1201; a strap (or band) connector 1202; an enclosure 1203; a plurality of spectroscopic sensors (1207, 1208, and 1209); and a plurality of spring mechanisms (1204, 1205, and 1206) which are configured to push the sensors inward toward the center of the device. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

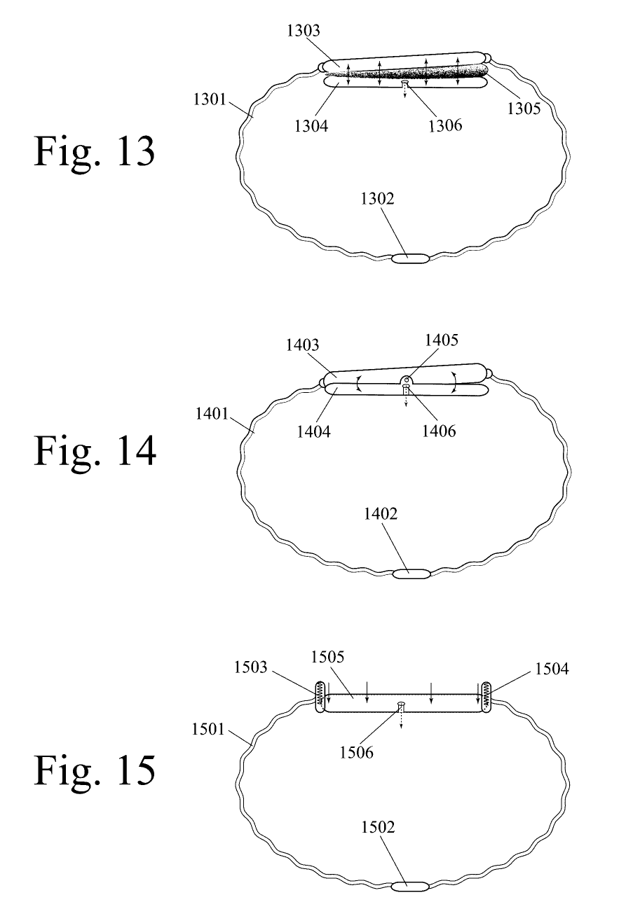

FIG. 13 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 13 is an arcuate wrist-worn device with a biometric sensor which is located on a circumference-center-facing portion of an enclosure, wherein this circumference-center-facing portion tilts on a central inflated portion of the enclosure so that the sensor remains in close contact with the surface of a person's arm even if the device tilts with respect to the arm surface. In this example, an enclosure is positioned on the anterior (upper) portion of the device circumference. In this example, the enclosure has an outward-facing portion (which can include a display screen), a central inflated portion (which can be a balloon), and an inner-facing portion (which houses the biometric sensor). In an example, a central inflated portion can be sandwiched between a rigid outward-facing portion and a rigid circumference-center-facing portion. In an example, the circumference-center-facing portion can tilt with respect to the outward-facing portion as the device tilts with respect to the surface of the person's arm.

Having a biometric sensor located on a circumference-center-facing portion of an enclosure which tilts on a central inflated portion can help to keep the biometric sensor in close proximity to the surface of the person's arm and at substantially the same angle with respect to the surface of a person's arm. This can be particularly important for a spectroscopic sensor, wherein it is desirable to maintain the same projection angle (and/or reflection angle) of a beam of light which is directed toward (and/or reflected from) the surface of a person's arm.

More specifically, the example shown in FIG. 13 is a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member, wherein this enclosure further comprises a rigid outward facing portion, an inflated central portion, and a rigid circumference-center-facing portion, wherein the rigid circumference-center-facing portion tilts relative to the rigid outward facing portion; and (c) a biometric sensor in the circumference-center-facing portion which is configured to record biometric data concerning the person's arm tissue.

In an example, there can be a display screen on the outward-facing surface of the enclosure. In an example, the central portion of an enclosure can be filled with a liquid or gel rather than inflated with a gas. In an example, there can be more than one biometric sensor on the rigid circumference-center-facing portion. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 13 includes: strap (or band) 1301, strap (or band) connector 1302, outward facing portion 1303 of an enclosure, circumference-center-facing portion 1304 of the enclosure, inflated central portion 1305 of the enclosure, and a biometric sensor 1306 on the circumference-center-facing portion of the enclosure. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 14 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 14 is an arcuate wrist-worn device with a biometric sensor which is located on a circumference-center-facing portion of an enclosure, wherein this circumference-center-facing portion pivots around an axis so that the sensor remains in close contact with the surface of a person's arm even if the device tilts with respect to the arm surface. In this example, an enclosure is positioned on the anterior (upper) portion of the device circumference. In this example, the enclosure has an outward-facing portion (which can include a display screen) and an inner-facing portion (which houses the biometric sensor).

In this example, a circumference-center-facing portion which houses a biometric sensor pivots around a central axis when the device tilts with respect to the surface of the person's arm. Having a biometric sensor located on a circumference-center-facing portion of an enclosure which pivots around an axis can help to keep the biometric sensor in close proximity to the surface of the person's arm and at substantially the same angle with respect to the surface of a person's arm. This can be particularly important for a spectroscopic sensor, wherein it is desirable to maintain the same projection angle (and/or reflection angle) of a beam of light which is directed toward (and/or reflected from) the surface of a person's arm.

More specifically, the example shown in FIG. 14 is a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member, wherein this enclosure further comprises an outward facing portion and a circumference-center-facing portion, wherein the rigid inward (or center) pivots around a central axis with respect to the outward facing portion; and (c) a biometric sensor in the circumference-center-facing portion which is configured to record biometric data concerning the person's arm tissue.

In this example, the central axis around which the circumference-center-facing portion pivots is perpendicular to the circumference of the device.In another example, the central axis around which the circumference-center-facing portion pivots can be parallel or tangential to the circumference of the device. In an example, there can be a display screen on the outward-facing surface of the enclosure. In an example, there can be more than one biometric sensor on the circumference-center-facing portion of the enclosure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 14 includes: strap (or band) 1401, strap (or band) connector 1402, outward facing portion 1403 of an enclosure, circumference-center-facing portion 1404 of the enclosure, axis 1405 around which circumference-center-facing portion 1404 pivots; and a biometric sensor 1406 on the circumference-center-facing portion of the enclosure. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 15 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 15 is a wrist-worn device with a biometric sensor located on an enclosure, wherein the enclosure is pushed toward the surface of a person's arm by spring mechanisms so that the sensor remains in close contact with the arm's surface even if the rest of the device shifts away from the arm's surface. In this example, the enclosure is on the anterior (upper) portion of the device circumference.

The example shown in FIG. 15 can also be expressed as a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member; (c) one or more spring mechanisms which push the enclosure inward toward the circumference center of the device; and (d) a biometric sensor in the enclosure which is configured to record biometric data concerning the person's arm tissue.

In this example, there are two spring mechanisms which push the enclosure inward toward the surface of a person's arm. In this example, these spring mechanisms are located at the places where the enclosure is connected to a strap or band. In an example, there can be a display screen on the outward-facing surface of the enclosure. In an example, there can be more than one biometric sensor on the circumference-center-facing portion of the enclosure. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 15 includes: strap (or band) 1501, strap (or band) connector 1502, first spring mechanism 1503, second spring mechanism 1504, enclosure 1505 which is pushed inward (toward the circumference center of the device) by spring mechanisms 1503 and 1504, and biometric sensor 1506. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

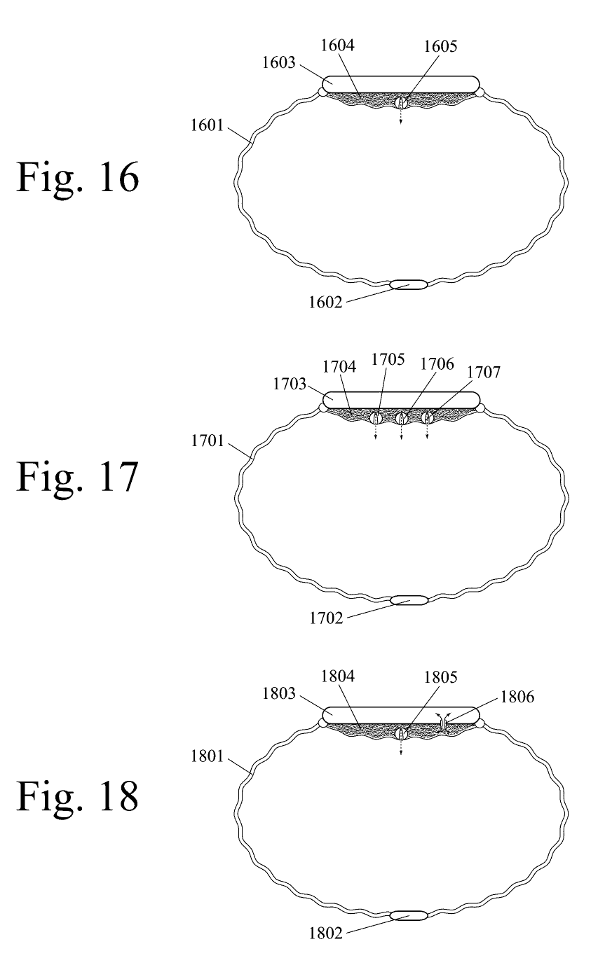

FIG. 16 shows another example of a wearable device for the arm with one or more close-fitting biometric sensors. This figure shows the device from a side perspective, as it would appear encircling a lateral cross-section of a person's wrist (or other portion of the person's arm). Described generally, this example is a wrist-worn device with an elastic member (such as a balloon) that is filled with a fluid, gel, or gas and a biometric sensor which is attached to the circumference-center-facing wall of this elastic member. Having a biometric sensor attached to the circumference-center-facing wall of an elastic member can help to keep the sensor in close contact with the surface of a person's arm, even if other components of the device are shifted or tilted away from the arm's surface. In an example, an elastic member can be part of an enclosure which is attached to an arm by a strap. In an example, such an enclosure can be positioned on the anterior (upper) portion of the device circumference.

The example shown in FIG. 16 can also be expressed as a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member; (c) an elastic member filled with a fluid, gel, or gas which is attached to (or part of) the enclosure; and (d) a biometric sensor which is configured to record biometric data concerning the person's arm tissue, wherein this sensor is attached to a circumference-center-facing wall of the elastic member.

In an example, there can be a display screen on the outward facing surface of an enclosure. In an example, there can be more than one biometric sensor on the circumference-center-facing wall of an elastic member. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 16 includes: strap (or band) 1601; strap (or band) connector 1602; enclosure 1603; elastic member 1604 which is filled with a fluid, gel, or gas; and biometric sensor 1605 which is attached to the circumference-center-facing wall of the elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 17 shows another example of a wearable device for the arm with one or more close-fitting biometric sensors. This figure shows the device from a side perspective, as it would appear encircling a lateral cross-section of a person's wrist (or other portion of the person's arm). The example shown in FIG. 17 is like the one shown in FIG. 16, except that in FIG. 17 there are multiple biometric sensors on the circumference-center-facing wall of an elastic member. In FIG. 17, there are three biometric sensors.

With respect to specific components, the example shown in FIG. 17 includes: strap (or band) 1701; strap (or band) connector 1702; enclosure 1703; elastic member 1704 which is filled with a fluid, gel, or gas; and biometric sensors 1705, 1706, and 1707 which are attached to the circumference-center-facing wall of the elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 18 shows another example of a wearable device for the arm with one or more close-fitting biometric sensors. This figure shows the device from a side perspective, as it would appear encircling a lateral cross-section of a person's wrist (or other portion of the person's arm). The example shown in FIG. 18 is like the one shown in FIG. 16, except that in FIG. 18 there is also a micropump which can pump fluid, gel, or gas into (or out of) the elastic member. This enables (automatic) adjustment of the size and/or internal pressure of the elastic member in order to better maintain proximity of the sensor to the surface of the person's arm.

With respect to specific components, the example shown in FIG. 18 includes: strap (or band) 1801; strap (or band) connector 1802; enclosure 1803; elastic member 1804 which is filled with a fluid, gel, or gas; biometric sensor 1805 which is attached to the circumference-center-facing wall of the elastic member; and micropump 1806 which pumps fluid, gel, or gas into (or out of) the elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

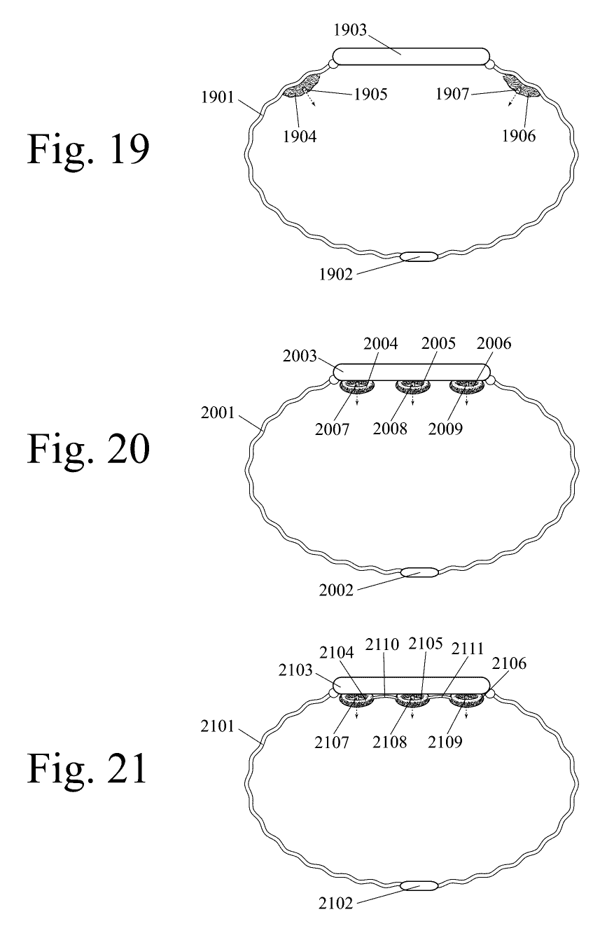

FIG. 19 shows another example of a wearable device for the arm with one or more close-fitting biometric sensors. This figure shows the device from a side perspective, as it would appear encircling a lateral cross-section of a person's wrist (or other portion of the person's arm). This wrist-worn device comprises: an attachment member which is configured to span at least a portion of the circumference of a person's arm; one or more elastic members filled with a flowable substance, wherein these elastic members are part of (or attached to) the circumference-center-facing surface of the attachment member; and one or more biometric sensors, wherein each sensor is part of (or attached to) a circumference-center-facing wall of an elastic member.

The design of this device keeps biometric sensors close to the surface of a person's arm, even if portions of the device shift away from the surface of the person's arm. The interiors of the elastic members on which these sensors are located are under modest pressure so that these elastic members expand when they are moved away from the arm surface and these elastic members are compressed when they are moved toward the arm surface.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, an attachment member can be attached to a person's arm by stretching it circumferentially and sliding it over the person's hand onto the arm. In an example, an attachment member can be attached to a person's arm by applying force to pull two ends of the member apart in order to slip the member over the arm; the two ends then retract back towards each other when device is on the arm and the force is removed.

In an example, an elastic member can be a balloon or other elastic substance-filled compartment. In an example, the flowable substance inside an elastic member can be a fluid, gel, or gas. In this example, there are two elastic members on the attachment member. In this example, the elastic members are symmetrically located with respect to a central cross-section of the device. In an example, there can be a plurality of elastic members (with attached biometric sensors) which are distributed around the circumference of an attachment member and/or the device. In this example, a device can also include an enclosure which further comprises a display screen.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 19 includes: band 1901; band connector 1902; enclosure 1903; first elastic member 1904 which is filled with a fluid, gel, or gas; first biometric sensor 1905 which is attached to the circumference-center-facing wall of the first elastic member; second elastic member 1906 which is filled with a fluid, gel, or gas; and second biometric sensor 1907 which is attached to the circumference-center-facing wall of the second elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 20 shows another example of a wearable device for the arm with one or more close-fitting biometric sensors. This figure shows the device from a side perspective, as it would appear encircling a lateral cross-section of a person's wrist (or other portion of the person's arm). This wrist-worn device comprises: (a) an attachment member which is configured to span at least a portion of the circumference of a person's arm; (b) an enclosure which is part of (or attached to) the attachment member; (c) one or more torus-shaped elastic members filled with a flowable substance, wherein these elastic members are part of (or attached to) the enclosure; and (d) one or more biometric sensors, wherein each sensor is located in the central hole of a torus-shaped elastic member.

In an example, an attachment member can be a strap, band, bracelet, ring, armlet, cuff, or sleeve. In an example, an enclosure can further comprise a display screen on its outer surface. In an example, a torus-shaped elastic member can be a balloon which is filled with a fluid, gel, or gas. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 20 includes: band 2001; band connector 2002; enclosure 2003; torus-shaped elastic members 2004, 2005, and 2006; and biometric sensors 2007, 2008, and 2009 which are each located in the central opening (or hole) of a torus-shaped elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 21 shows another example of a wearable device for the arm with one or more close-fitting biometric sensors. This figure shows the device from a side perspective, as it would appear encircling a lateral cross-section of a person's wrist (or other portion of the person's arm). The example in FIG. 21 like the one shown in FIG. 20, except that the example in FIG. 21 also includes channels through which a fluid, gel, or gas can flow between the torus-shaped elastic members.

With respect to specific components, the example shown in FIG. 21 includes: band 2101; band connector 2102; enclosure 2103; torus-shaped elastic members 2104, 2105, and 2106; biometric sensors 2107, 2108, and 2109 which are each located in the central opening (or hole) of a torus-shaped elastic member; and channels 2110 and 2111 through which fluid, gel, or gas can flow between the torus-shaped elastic members. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 22 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 22 is an arcuate wrist-worn device with a light-projecting spectroscopic sensor on a rotating ball. Rotating the ball changes the angle at which the spectroscopic sensor projects light onto the surface of a person's arm. The ball can be rotated in different directions so that the range of possible projection beams comprises a conic or frustal shape in three-dimensional space. Having a light-projecting spectroscopic sensor on a rotating ball can enable a device to record biometric measurements with substantially the same angle of incidence, even if an enclosure is tilted with respect to the surface of the person's arm.

The example shown in FIG. 22 is a wearable device for the arm with a close-fitting biometric sensor comprising: (a) an attachment member, such as a strap or band, which is configured to span at least a portion of the circumference of a person's arm; (b) an enclosure which is part of (or attached to) the attachment member; (c) a rotating ball which is part of (or attached to) the enclosure; and (d) a light-projecting spectroscopic sensor which is part of (or attached to) the rotating ball.

In an example, an attachment member can be a strap, band, bracelet, ring, armlet, cuff, or sleeve. In an example, the circumference-center-facing surface of an enclosure can be substantially flat. In an example, the circumference-center-facing surface of an enclosure can be curved. In an example, there can be a display screen on the outward-facing surface of an enclosure. In an example, the rotating ball can fit into the enclosure like a ball-and-socket joint. In an example, the device can further comprise one or more actuators which move the rotating ball.

With respect to specific components, the example shown in FIG. 22 includes: strap 2201, strap connector 2202, enclosure 2203, rotating ball 2204, and spectroscopic sensor 2205 which emits beam of light 2206. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

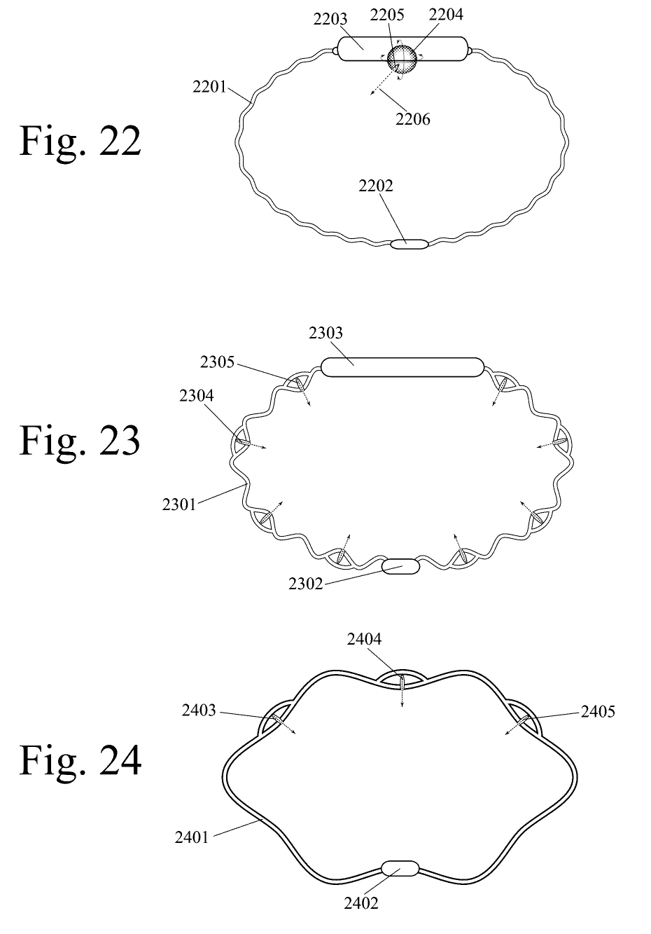

FIG. 23 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 23 is a wearable device for the arm with a flexible circumferentially-undulating band with biometric sensors on the proximal portions of undulating waves. A band with such a flexible circumferentially-undulating structure can help to keep a plurality of biometric sensors in close proximity to the surface of a person's arm. In an example, an attachment member can be a strap, band, bracelet, ring, or armlet. In an example, a circumferentially-undulating attachment member can have a repeating wave pattern. In an example, a circumferentially-undulating attachment member can have a sinusoidal wave pattern.

The example shown in FIG. 23 is a wearable device for the arm with a close-fitting biometric sensor comprising: (a) a circumferentially-undulating attachment member which is configured to span at least a portion of the circumference of a person's arm; and (b) a plurality of biometric sensors which collect data concerning arm tissue, wherein each biometric sensor is located at the proximal portion of an undulation, and wherein the proximal portion of an undulation is the portion of an undulating wave which is closest to the circumferential center of the device.

With respect to specific components, the example shown in FIG. 23 includes: circumferentially-undulating band 2301, band connector 2302, enclosure 2303, first biometric sensor 2304 at the proximal portion of a first wave in the circumferentially-undulating band, and second biometric sensor 2305 at the proximal portion of a second wave in the circumferentially-undulating band. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 24 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 24 is a wearable device for the arm with a flexible circumferentially-undulating band with six waves and biometric sensors on the proximal portions of some or all of these waves.

A band with a circumferentially-undulating structure can help to keep a plurality of biometric sensors in close proximity to the surface of a person's arm. Further, a band with six waves can engage the sides of a person's wrist with two symmetrically-opposite waves to resist rotational shifting better than a circular or oval band. This can help to reduce measurement errors caused by movement of biometric sensors. In an example, a circumferentially-undulating attachment member can be a strap, band, bracelet, ring, or armlet. In an example, a circumferentially-undulating attachment member can have a repeating wave pattern. In an example, a circumferentially-undulating attachment member can have a sinusoidal wave pattern.

The example shown in FIG. 24 is a wearable device for the arm with one or more close-fitting biometric sensors comprising: (a) a circumferentially-undulating attachment member with six waves which is configured to span the circumference of a person's arm; and (b) a plurality of biometric sensors which collect data concerning arm tissue, wherein each biometric sensor is located at the proximal portion of an undulation, and wherein the proximal portion of an undulation is the portion of an undulating wave which is closest to the circumferential center of the device.

With respect to specific components, the example shown in FIG. 24 includes: circumferentially-undulating band 2401 with six waves, band connector 2402, a first biometric sensor 2403 at the proximal portion of a first wave in the circumferentially-undulating band, a second biometric sensor 2405 at the proximal portion of a second wave in the circumferentially-undulating band, and a third biometric sensor 2406 at the proximal portion of a third wave in the circumferentially-undulating band. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

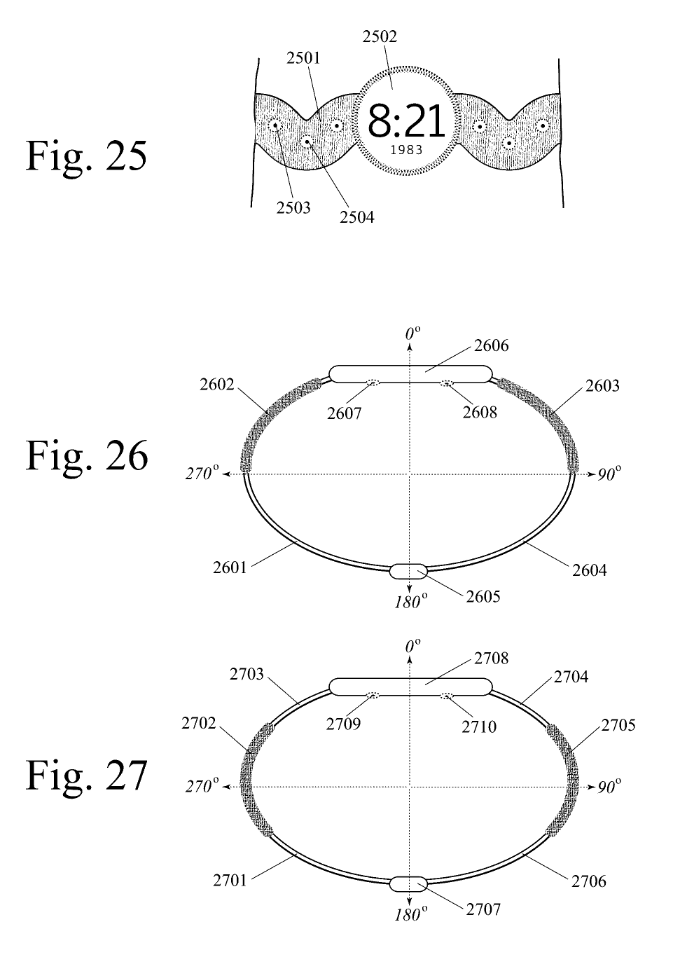

FIG. 25 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. Described generally, the example shown in FIG. 25 is a wearable device for the arm with a laterally-undulating band and biometric sensors. Lateral undulations are waves which are substantially perpendicular to the plane containing the band circumference. In an example, a band can have sinusoidal lateral undulations.

The example shown in FIG. 25 is a wearable device for the arm with one or more close-fitting biometric sensors comprising: (a) a laterally-undulating attachment member which is configured to span at least a portion of the circumference of a person's arm, wherein lateral undulations are waves which are substantially perpendicular to the plane containing the circumference of the attachment member; and (b) one or more biometric sensors which collect data concerning arm tissue, wherein these biometric sensors are part of (or attached to) the laterally-undulating attachment member.

With respect to specific components, the example shown in FIG. 25 includes: laterally-undulating strap 2501; display screen 2502; and biometric sensors including 2503 and 2504. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 26 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 26 is a wearable device for an arm with one or more biometric sensors in an enclosure and an attachment member (such as a strap, band, bracelet, or cuff) which attaches the enclosure to the arm, wherein this attachment member has relatively-elastic portions connected to the enclosure and relatively-inelastic portions elsewhere. This structure can help to keep the enclosure and sensors fitting closely against the arm. This, in turn, can enable more-consistent collection of data concerning arm tissue.

In an example, the device in FIG. 26 can be specified as a wearable device for the arm with one or more close-fitting biometric sensors comprising: (a) an attachment member which is configured to span at least 60% of the circumference of a person's arm, wherein this attachment member further comprises—one or more elastic portions which are configured to span the anterior (upper) surface of a person's arm and one or more inelastic portions which are configured to span the posterior (lower) surface of the person's arm; (b) an enclosure which is connected to the elastic portions of the attachment member; and (c) one or more biometric sensors which collect data concerning arm tissue which are part of (or attached to) the enclosure.

In an alternative example, a wearable device for the arm with one or more close-fitting biometric sensors can comprise: (a) an attachment member which is configured to span at least 60% of the circumference of a person's arm, wherein this attachment member further comprises—one or more elastic portions which are configured to span the posterior (lower) surface of a person's arm and one or more inelastic portions which are configured to span the anterior (upper) surface of the person's arm; (b) an enclosure which is connected to the elastic portions of the attachment member; and (c) one or more biometric sensors which collect data concerning arm tissue which are part of (or attached to) the enclosure.

In an example, an elastic portion of an attachment member can be an elastic strap or band. In an example, an elastic portion of an attachment member can be made from elastic fabric. In an example, an elastic portion of an attachment member can have a first elasticity level, an inelastic portion of an attachment member can have a second elasticity level, and the first elasticity level can be greater than the second elasticity level. In an example, a first elastic portion of an attachment member can be directly connected to a first side of an enclosure and a second elastic portion of an attachment member can be directly connected to a second (opposite) side of the enclosure. In an example, a first elastic portion of an attachment member can be indirectly connected to a first side of an enclosure and a second elastic portion of an attachment member can be indirectly connected to a second (opposite) side of the enclosure.

In an example, the device in FIG. 26 can be specified as a wearable device for the arm with one or more close-fitting biometric sensors comprising: (a) an attachment member which is configured to span at least 60% of the circumference of a person's arm wherein this attachment member further comprises: two elastic portions which are configured to span a first portion of the circumference of a person's arm; and two inelastic portions which are configured to span a second portion of the circumference of the person's arm; (b) an enclosure which is connected between the two elastic portions; (c) a clip, buckle, clasp, pin, or hook-and-eye mechanism between the two inelastic portions; and d) one or more biometric sensors which collect data concerning arm tissue which are part of (or attached to) the enclosure.

In an example, the device in FIG. 26 can be specified as a wearable device for the arm with one or more close-fitting biometric sensors comprising: (a) an attachment member which is configured to span at least 60% of the circumference of a person's arm wherein this attachment member further comprises: two elastic portions of the attachment member which are configured to span a portion of the circumference of a person's arm; and one or more inelastic portions which comprise the remainder of the attachment member; (b) an enclosure which is connected between the two elastic portions; (c) one or more biometric sensors which collect data concerning arm tissue which are part of (or attached to) the enclosure.

In an example, a single elastic portion can be configured to span at least 10% of the circumference of a person's arm. In an example, a single elastic portion can be configured to span at least 10% of the circumference of an attachment member. In an example, a single inelastic portion can be configured to span at least 10% of the circumference of a person's arm. In an example, a single inelastic portion can be configured to span at least 10% of the circumference of an attachment member. In an example, two elastic portions can be configured to collectively span at least 20% of the circumference of a person's arm. In an example, two elastic portions can be configured to collectively span at least 20% of the circumference of an attachment member. In an example, two inelastic portions can be configured to collectively span at least 20% of the circumference of a person's arm. In an example, two inelastic portions can be configured to collectively span at least 20% of the circumference of an attachment member.

In an example, a first definition of polar (or compass) coordinates can be defined for a device relative to how the device is configured to be worn on a person's arm. A 0-degree position can be defined as the position on a device circumference which is configured to intersect the longitudinal mid-line of the anterior (upper) surface of the arm. A 180-degree position is diametrically opposite (through the circumferential center) the 0-degree position. A 90 degree position is (clockwise) midway between the 0-degree and 180-degree positions. A 270-degree position is diametrically opposite the 90 degree position.

Using this first definition of polar coordinates, the device in FIG. 26 can be specified as a wearable device for the arm with one or more close-fitting biometric sensors comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90 degree positions, an inelastic third portion with a third level of elasticity which spans at least 35 degrees (clockwise) between the 90 degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is configured to be worn (clockwise) between the 270-degree and 90 degree positions; and (c) one or more biometric sensors which collect data concerning arm tissue which are part of (or attached to) the enclosure.

Using this first definition of polar coordinates, the device in FIG. 26 can also be specified as a wearable device for the arm with one or more close-fitting biometric sensors comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90 degree positions, an inelastic third portion with a third level of elasticity which spans at least 35 degrees (clockwise) between the 90 degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is connected between the elastic first and second portions; and (c) one or more biometric sensors which collect data concerning arm tissue which are part of (or attached to) the enclosure.

Alternatively, a second definition of polar (or compass) coordinates can be defined for the circumference of such a device relative to the position of an enclosure. The 0-degree position can be defined as the position on the device circumference which intersects the (lateral) mid-line of the enclosure. The 180-degree position is diametrically opposite (through the circumferential center) the 0-degree position. The 90 degree position is clockwise midway between the 0-degree and 180-degree positions. The 270-degree position is diametrically opposite the 90 degree position.

Using this second definition of polar coordinates, the device in FIG. 26 can be specified as a wearable device for the arm with one or more close-fitting biometric sensors comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90 degree positions, an inelastic third portion with a third level of elasticity which spans at least 35 degrees (clockwise) between the 90 degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is connected between the elastic first and second portions of the attachment member; and (c) one or more biometric sensors which collect data concerning arm tissue which are part of (or attached to) the enclosure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 26 includes: inelastic portion 2601 of an attachment member; elastic portion 2602 of an attachment member; elastic portion 2603 of an attachment member; inelastic portion 2604 of an attachment member; attachment member connector 2605; enclosure 2606; and biometric sensors 2607 and 2608. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 27 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 27 is a wearable device for the arm with one or more biometric sensors in an enclosure and an attachment member (such as a strap, band, bracelet, or cuff) which attaches the enclosure to the arm, wherein the attachment member is configured to have elastic portions spanning the lateral surfaces of the arm and inelastic portions spanning the anterior (upper) and posterior (lower) surfaces of the arm. This structure can help to keep the enclosure and sensors from rotating around the arm. This, in turn, can enable more-consistent collection of data concerning arm tissue.

In an example, the device in FIG. 27 can be specified as a wearable device for the arm with one or more close-fitting biometric sensors comprising: (a) an attachment member which is configured to span at least 60% of the circumference of a person's arm wherein this attachment member further comprises—one or more anterior inelastic portions which are configured to span the anterior (upper) surface of a person's arm, one or more posterior inelastic portions which are configured to span the posterior (lower) surface of a person's arm, and one or more elastic portions which connect the anterior and posterior inelastic portions; (b) an enclosure which is configured to be worn on the anterior (upper) portion of the arm; and (c) one or more biometric sensors which collect data concerning arm tissue which are part of (or attached to) the enclosure.

In another example, a wearable device for the arm with one or more close-fitting biometric sensors can comprise: (a) an attachment member which is configured to span at least 60% of the circumference of a person's arm wherein this attachment member further comprises—one or more anterior inelastic portions which are configured to span the anterior (upper) surface of a person's arm, one or more posterior inelastic portions which are configured to span the posterior (lower) surface of a person's arm, and one or more elastic portions which connect the anterior and posterior inelastic portions; (b) an enclosure which is configured to be worn on the posterior (lower) portion of the arm; and (c) one or more biometric sensors which collect data concerning arm tissue which are part of (or attached to) the enclosure.

In an example, a first inelastic portion of an attachment member can be connected to a first side of an enclosure and a second inelastic portion of an attachment member can be connected to a second side of the enclosure. In an example, an elastic portion can have a first level of elasticity, an inelastic portion can have a second level of elasticity, and the first level is greater than the second level. In an example, a single elastic portion can be configured to span at least 10% of the circumference of a person's arm. In an example, a single elastic portion can be configured to span at least 10% of the circumference of an attachment member. In an example, a single inelastic portion can be configured to span at least 10% of the circumference of a person's arm. In an example, a single inelastic portion can be configured to span at least 10% of the circumference of an attachment member.

In an example, polar (or compass) coordinates can be defined for a device relative to how the device is configured to be worn on a person's arm. A 0-degree position can be defined as the position on a device circumference which is configured to intersect the longitudinal mid-line of the anterior (upper) surface of the arm. A 180-degree position is diametrically opposite (through the circumferential center) the 0-degree position. A 90 degree position is clockwise midway between the 0-degree and 180-degree positions. A 270-degree position is diametrically opposite the 90 degree position.

In an example, the device in FIG. 27 can be specified as a wearable device for the arm with one or more close-fitting biometric sensors comprising: (a) an attachment member which is configured to span at least 60% of the circumference of a person's arm wherein this attachment member further comprises—a inelastic first portion with a first level of elasticity which spans at least 35 degrees (clockwise) between the 270-degree and 90 degree positions; an inelastic second portion with a second level of elasticity which spans at least 35 degrees (clockwise) between the 90 degree and 270-degree positions, an elastic third portion with a third level of elasticity which spans at least 35 degrees (clockwise) between the 180-degree and 0-degree positions, an elastic fourth portion with a fourth level of elasticity which spans at least 35 degrees (clockwise) between the 0-degree and 180-degree positions, and wherein each of the first and second elasticity levels is lower than each of the third and fourth elasticity levels; (b) an enclosure that is connected between the inelastic first portion and the inelastic second portion; and (c) one or more biometric sensors which collect data concerning arm tissue which are part of (or attached to) the enclosure.

In an alternative example, polar (or compass) coordinates can be defined for the circumference of such a device relative to the position of an enclosure on the device. The 0-degree position can be defined as the position on the device circumference which intersects the (lateral) mid-line of the enclosure. The 180-degree position is diametrically opposite (through the circumferential center) the 0-degree position. The 90 degree position is clockwise midway between the 0-degree and 180-degree positions. The 270-degree position is diametrically opposite the 90 degree position.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 27 includes: inelastic portion 2701 of an attachment member; elastic portion 2702 of an attachment member; inelastic portion 2703 of an attachment member; inelastic portion 2704 of an attachment member; elastic portion 2705 of an attachment member; inelastic portion 2706 of an attachment member; attachment member connector 2707; enclosure 2708; and biometric sensors 2709 and 2710. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 28 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 28 can be described as an arm-wearable device with a relatively-rigid band and a relatively-elastic band, wherein each of these bands spans at least 60% of the circumference of a person's arm, wherein these bands are connected to each other, and wherein there are biometric sensors on the relatively-elastic band.

More specifically, the example shown in FIG. 28 is a wearable device for the arm with one or more close-fitting biometric sensors comprising: (a) an inelastic attachment member which is configured to span at least 60% of the circumference of a person's arm, wherein this inelastic attachment member has a first elasticity level; (b) an elastic attachment member which is configured to span at least 60% of the circumference of a person's arm, wherein this elastic attachment member has a second elasticity level, wherein the second elasticity level is greater than the first elasticity level, and wherein the elastic attachment member is connected to the inelastic attachment member; and (c) one or more biometric sensors which are configured to collect data concerning arm tissue, wherein these biometric sensors are part of (or attached to) the elastic attachment member.

In an example, an attachment member can be a band, ring, strap, bracelet, bangle, armlet, sleeve, or cuff. In an example, a band or other attachment member can be attached to a person's arm by connecting two ends of the attachment member with a clasp, clip, buckle, hook, pin, plug, or hook-and-eye mechanism. In an example, a band or other attachment member can be attached to a person's arm by stretching and sliding it over the person's hand onto the arm. In an example, a band or other attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 28 include: inelastic band 2801; elastic band 2802; display screen 2803; and biometric sensors including 2804.

FIG. 29 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 29 can be described as an arm-wearable device with two or more modular and connectable bands, wherein each band spans at least 60% of the circumference of a person's arm, and wherein one or more of these bands house biometric sensors.

More specifically, the example shown in FIG. 29 is a wearable device for the arm with one or more close-fitting biometric sensors comprising: (a) a first modular band which is configured to span at least 60% of the circumference of a person's arm; (b) a second modular band which is configured to span at least 60% of the circumference of a person's arm, wherein the first modular band and the second modular band have a first configuration in which they are not connected to each other and are not worn by a person, wherein the first band and the second band have a second configuration wherein they are connected to each other and worn on a person's arm, and wherein the first band and the second band can be changed from the first configuration to the second configuration by the person who wears them, and wherein the first band and the second band can be changed back from the second configuration to the first configuration by the person who wears them; and (c) one or more biometric sensors which are configured to collect data concerning arm tissue, wherein these biometric sensors are part of (or attached to) one or both of the modular bands.

In an example, an attachment member can be a band, ring, strap, bracelet, bangle, armlet, sleeve, or cuff. In an example, a band or other attachment member can be attached to a person's arm by connecting two ends of the attachment member with a clasp, clip, buckle, hook, pin, plug, or hook-and-eye mechanism. In an example, a band or other attachment member can be attached to a person's arm by stretching and sliding it over the person's hand onto the arm. In an example, a band or other attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 29 include: first modular band 2901; second modular band 2902; temporary connectors 2903 and 2904; and display screens 2905 and 2906.

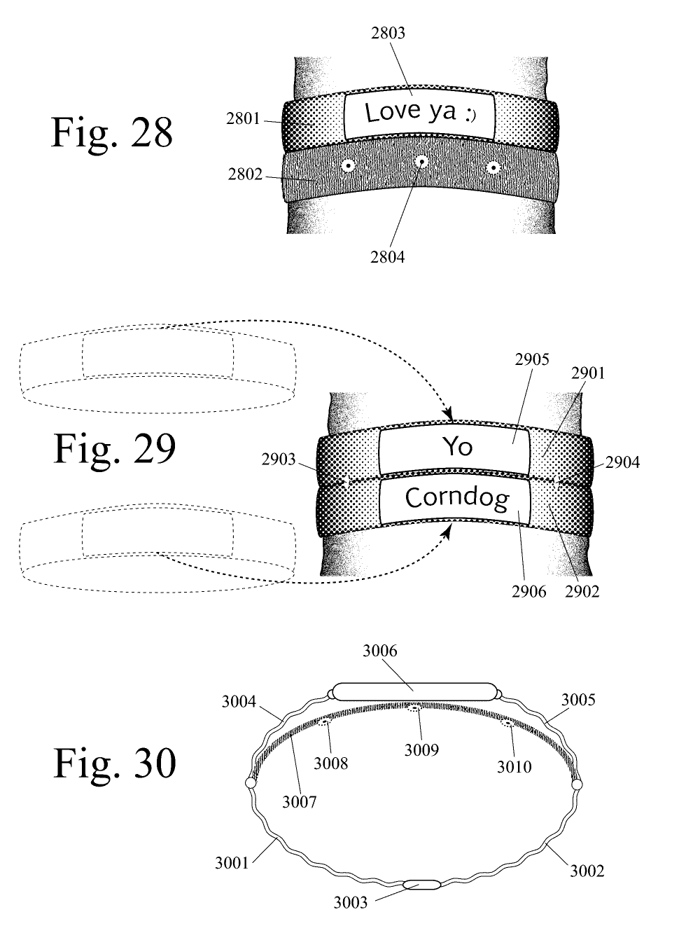

FIG. 30 shows another example of a wearable device for the arm with one or more close-fitting biometric sensors. This figure shows the device from a side perspective, as it would appear encircling a lateral cross-section of a person's arm.

The example in FIG. 30 can be described as an arm-wearable device with a partial-circumferential inner elastic band and biometric sensors. Such a device can have an outer inelastic band with a first elasticity level which spans a first percentage of the arm circumference and an inner elastic band with a second elasticity level which spans a second percentage of the arm circumference—wherein the second percentage is less than the first percentage and the second elasticity level is greater than the first elasticity level. In the example shown in FIG. 30, an outer inelastic band (and display screen) spans the entire arm circumference and a semi-circular inner elastic band (interior relative to the outer inelastic band) spans only half of the arm circumference. This design can provide an overall semi-rigid structure (for housing a display screen), but can also keep biometric sensors close against the surface of the arm for consistent collection of biometric data.

More specifically, the example shown in FIG. 30 is a wearable device for the arm with one or more close-fitting biometric sensors comprising: (a) an outer inelastic band which is configured to span a first percentage of a person's arm and which has a first elasticity level; (b) an inner elastic band which is configured to span a second percentage of a person's arm and which has a second elasticity level, wherein this inner elastic band is configured to be closer to the surface of the arm than the outer inelastic band, wherein the second percentage is less than the first percentage, and wherein the second elasticity level is greater than the first elasticity level; and (c) one or more biometric sensors which are configured to collect data concerning arm tissue, wherein these biometric sensors are part of (or attached to) the inner elastic band.

Alternatively, the example shown in FIG. 30 can be specified as a wearable device for the arm with one or more close-fitting biometric sensors comprising: (a) an outer inelastic band with a first arcuate length and a first elasticity level; (b) an inner elastic band with a second arcuate length and a second elasticity level, wherein this inner elastic band is located on the concave side of the outer elastic band, wherein the second percentage is less than the first percentage, and wherein the second elasticity level is greater than the first elasticity level; and (c) one or more biometric sensors which are configured to collect data concerning arm tissue, wherein these biometric sensors are part of (or attached to) the inner elastic band.

In an example, the word “ring”, “strap”, “bracelet”, “bangle”, “armlet”, “sleeve”, or “cuff” can be substituted for the word “band” in the above specifications. In an example, an outer inelastic band can span Y % of the circumference of a person's arm and an inner elastic band can span X % of the circumference of a person's arm, wherein Y % is at least 20 percentage points greater than X %. In an example, Y % can be 75% and X % can be 50%. In an example, the ends of the inner elastic band can be attached to the outer inelastic band. In an example, an inner elastic band can be configured to span the anterior (upper) surface of a person's arm. In an example, an inner elastic band can be configured to span the posterior (lower) surface of a person's arm.

In an example, an outer inelastic band can be attached to a person's arm by connecting two ends of an outer inelastic band with a clasp, clip, buckle, hook, pin, plug, or hook-and-eye mechanism. In an example, an outer inelastic band can be attached to a person's arm by stretching and sliding it over the person's hand onto the arm. In an example, an outer inelastic band can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 30 include: four segments (3001, 3002, 3004, and 3005) of an outer inelastic band; inner elastic band 3007; biometric sensors (3008, 3009, and 3010); outer elastic band clasp 3003; and display screen 3006.

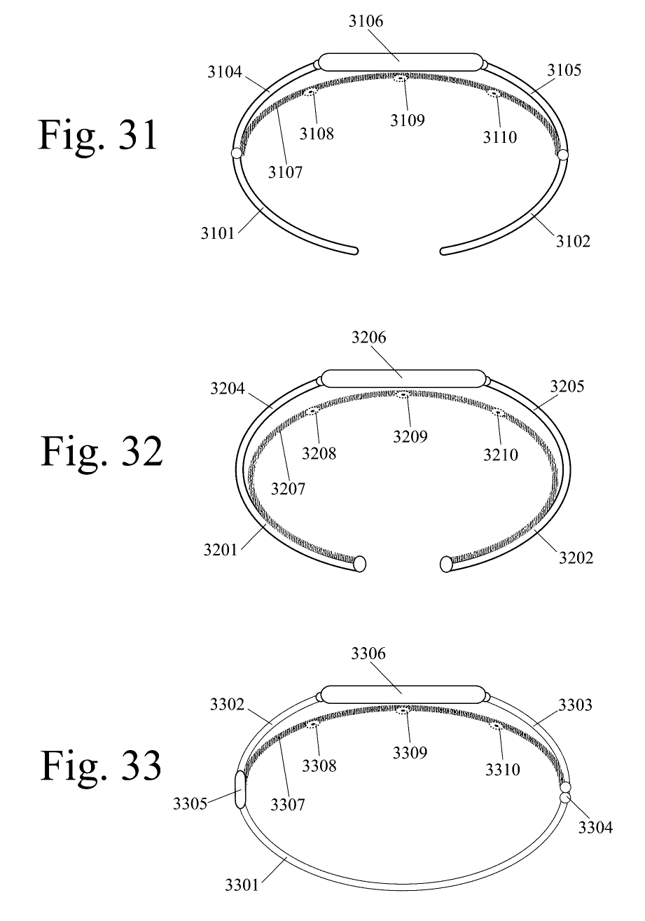

FIG. 31 shows another example of a wearable device for the arm with one or more close-fitting biometric sensors. This figure shows the device from a side perspective, as it would appear encircling a lateral cross-section of a person's wrist (or other portion of their arm). The example in FIG. 31 is like the one shown in FIG. 30, except that in FIG. 31 the outer inelastic band is sufficiently resilient that its ends hold onto the person's arm without the need for a clasp. The outer inelastic band can be attached to a person's arm by applying force to pull two ends apart to slip the member over the arm, wherein the two ends retract back towards each other when the force is removed.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 31 include: four segments (3101, 3102, 3104, and 3105) of an outer inelastic band; inner elastic band 3107; biometric sensors (3108, 3109, and 3110); and display screen 3106.

FIG. 32 shows another example of a wearable device for the arm with one or more close-fitting biometric sensors. This figure shows the device from a side perspective, as it would appear encircling a lateral cross-section of a person's arm. The example in FIG. 32 can be described as an arm-wearable device with an outer arcuate inelastic band, an inner arcuate elastic band, and biometric sensors which are part of the inner band. This design can provide an overall semi-rigid structure (e.g. to hold a rigid display screen in place) and also keep biometric sensors close against the surface of the arm for consistent collection of biometric data.

The example shown in FIG. 32 can be specified as a wearable device for the arm with one or more close-fitting biometric sensors comprising: (a) an outer arcuate inelastic band which is configured to span at least 60% of the circumference of a person's arm and which has a first elasticity level; (b) an inner arcuate elastic band which is located on (and attached to) the concave side of the outer arcuate band and which has a second elasticity level, wherein the second elasticity level is greater than the first elasticity level; and (c) one or more biometric sensors which are configured to collect data concerning arm tissue, wherein these biometric sensors are part of (or attached to) the inner arcuate elastic band. In various examples, a ring, strap, bracelet, bangle, armlet, sleeve, or cuff can be substituted for a band.

Alternatively, the example shown in FIG. 32 can be specified as a wearable device for the arm with one or more close-fitting biometric sensors comprising: (a) an outer arcuate inelastic band, wherein this outer arcuate inelastic band is configured to span at least 60% of the circumference of a person's arm, wherein this outer arcuate inelastic band is configured to be a first average distance from the surface of the person's arm, and wherein this outer arcuate inelastic band has a first elasticity level; (b) an inner arcuate elastic band, wherein this inner arcuate elastic band is attached to the outer arcuate inelastic band, wherein this inner arcuate elastic band is configured to be an second average distance from the surface of the person's arm, wherein this inner arcuate elastic band has a second elasticity level, wherein the second average distance is less than the first average distance, and wherein the second elasticity level is greater than the first elasticity level; and (c) one or more biometric sensors which are configured to collect data concerning arm tissue, wherein these biometric sensors are part of (or attached to) the inner arcuate elastic band. In various examples, a ring, strap, bracelet, bangle, armlet, sleeve, or cuff can be substituted for a band.

In an example, an outer arcuate inelastic band can be attached to a person's arm by connecting two ends of the outer inelastic band with a clasp, clip, buckle, hook, pin, plug, or hook-and-eye mechanism. In an example, an outer arcuate inelastic band can be attached to a person's arm by stretching and sliding it over the person's hand onto the arm. In an example, an outer arcuate inelastic band can be attached to a person's arm by applying force to pull two ends apart to slip the member over the arm, wherein the two ends retract back towards each other when the force is removed. In an example, an inner arcuate elastic band can be made from a stretchable fabric. In an example, an inner arcuate elastic band can be attached to an outer arcuate inelastic band at the ends of the arcuate inelastic band. In an example, an inner arcuate elastic band can be attached to an outer arcuate inelastic band near mid-points of segments of the outer arcuate inelastic band.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 32 include: four segments (3201, 3202, 3204, and 3205) of an outer inelastic band; inner elastic band 3207; biometric sensors (3208, 3209, and 3210); and display screen 3206.

FIG. 33 shows another example of a wearable device for the arm with one or more close-fitting biometric sensors. This figure shows the device from a side perspective, as it would appear encircling a lateral cross-section of a person's wrist (or other portion of the person's arm). The example in FIG. 33 can be described as an arm-wearable device with an outer rigid “clam shell” structure to hold a display screen in place and an inner arcuate elastic band to keep biometric sensors close against the surface of the arm.

The example shown in FIG. 33 can be specified as a wearable device for the arm with one or more close-fitting biometric sensors comprising: (a) a clam shell structure which is configured to span the circumference of a person's arm, wherein this clam shell structure further comprises: an upper half-circumferential portion, a lower half-circumferential portion, a joint (and/or hinge) between these portions on a first side of these portions, and a connector which reversibly connects these portions on a second side of these portions; (b) an arcuate elastic band which is located within the concavity of the clam shell structure and is attached to the clam shell structure; and (c) one or more biometric sensors which are configured to collect data concerning arm tissue, wherein these biometric sensors are part of (or attached to) the arcuate elastic band.

In an example, an upper half-circumferential portion of a clam shell structure can span the anterior (upper) surface of a person's arm and a lower half-circumferential portion of a clam shell structure can span the posterior (lower) surface of the person's arm. In an example, there can be a display screen on the outer surface of one or both portions of a clam shell structure. In an example, a connector which reversibly connects the upper and lower portions of a clam shell structure can be selected from the group consisting of: clasp, clip, buckle, hook, pin, plug, and hook-and-eye mechanism. In an example, an inner arcuate elastic band can be made from a stretchable fabric. In an example, an inner arcuate elastic band can be attached to an upper half-circumferential portion of a clam shell structure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 33 include: two segments 3302 and 3303 of an upper half-circumferential portion of a clam shell structure; a lower half-circumferential portion 3301 of the clam shell structure; a joint (or hinge) 3304 between the upper and lower portions of the clam shell structure; a reversible connector 3305 between the upper and lower portions of the clam shell structure; an inner elastic band 3307; biometric sensors 3308, 3309, and 3310; and display screen 3306.

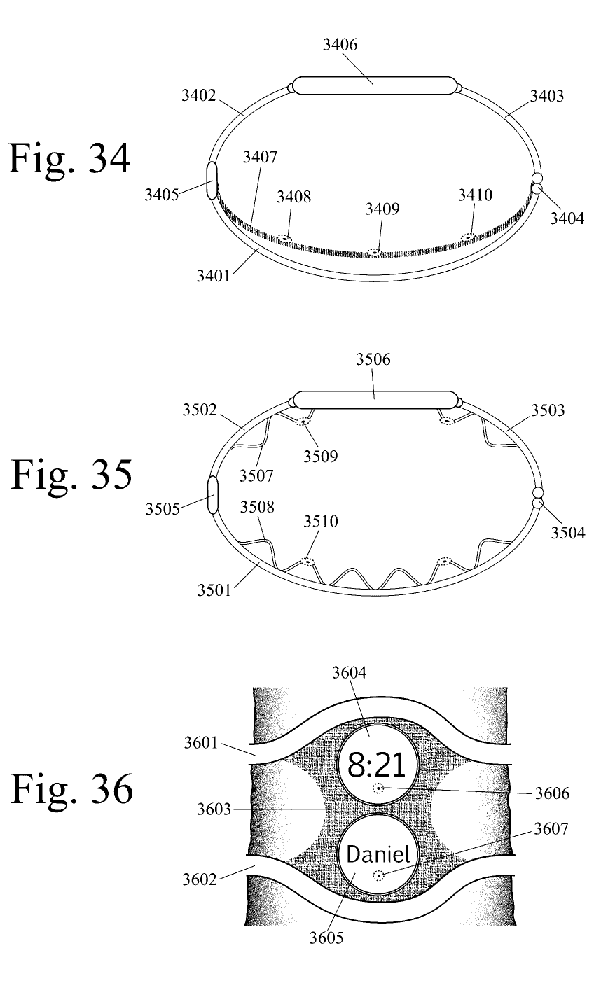

FIG. 34 shows another example of a wearable device for the arm with one or more close-fitting biometric sensors. This figure shows the device from a side perspective, as it would appear encircling a lateral cross-section of a person's wrist (or other portion of the person's arm). The example in FIG. 34 is like the one shown in FIG. 33, except that in FIG. 34 an inner arcuate elastic band spans the posterior (lower) surface of a person's arm. Specific components in the example shown in FIG. 34 include: two segments 3402 and 3403 of an upper half-circumferential portion of a clam shell structure; a lower half-circumferential portion 3401 of the clam shell structure; a joint (or hinge) 3404 between the upper and lower portions of the clam shell structure; a reversible connector 3405 between the upper and lower portions of the clam shell structure; an inner elastic band 3407; biometric sensors 3408, 3409, and 3410; and display screen 3406.

FIG. 35 shows another example of a wearable device for the arm with one or more close-fitting biometric sensors. This figure shows the device from a side perspective, as it would appear encircling a lateral cross-section of a person's wrist (or other portion of the person's arm). The example in FIG. 35 can be described as an arm-wearable device with an outer rigid “clam shell” structure and inward-facing flexible undulations to keep biometric sensors close against the surface of the arm.

The example shown in FIG. 35 can be specified as a wearable device for the arm with one or more close-fitting biometric sensors comprising: (a) a clam shell structure which is configured to span the circumference of a person's arm, wherein this clam shell structure further comprises: an upper half-circumferential portion, a lower half-circumferential portion, a joint (and/or hinge) between these portions on a first side of these portions, and a connector which reversibly connects these portions on a second side of these portions; (b) an inward-facing undulating member which is part of (or attached to) the clam shell structure; and (c) one or more biometric sensors which are configured to collect data concerning arm tissue, wherein these biometric sensors are part of (or attached to) the undulating member.

In an example, an upper half-circumferential portion of a clam shell structure can span the anterior (upper) surface of a person's arm and a lower half-circumferential portion of a clam shell structure can span the posterior (lower) surface of the person's arm. In an example, there can be a display screen on the outer surface of one or both portions of a clam shell structure. In an example, a connector which reversibly connects the upper and lower portions of a clam shell structure can be selected from the group consisting of: clasp, clip, buckle, hook, pin, plug, and hook-and-eye mechanism. In an example, an inward-facing undulating member can have a sinusoidal shape. In an example, an inward-facing undulating member can be flexible and/or compressible. In an example, an inward-facing undulating member can be elastic and filled with a liquid, gel, or gas.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 35 include: two segments 3502 and 3503 of an upper half-circumferential portion of a clam shell structure; a lower half-circumferential portion 3501 of the clam shell structure; a joint (or hinge) 3504 between the upper and lower portions of the clam shell structure; a reversible connector 3505 between the upper and lower portions of the clam shell structure; inward-facing undulating members including 3507 and 3508; biometric sensors including 3509 and 3510; and display screen 3506.

FIG. 36 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example in FIG. 36 can be described as an arm-wearable device with two display screens suspended by an elastic material between two arcuate bands.

The example shown in FIG. 36 can be specified as a wearable device for the arm with one or more close-fitting biometric sensors comprising: (a) a distal arcuate band which is configured to span at least 60% of the circumference of a person's arm; (b) a proximal arcuate band which is configured to span at least 60% of the circumference of a person's arm, wherein distal is defined as further from a person's shoulder and proximal is defined as closer to the person's shoulder; (c) an elastic member that is between the distal arcuate band and the proximal arcuate band which connects the distal actuate band to the proximal arcuate band; and (d) two arcuate display screens between the distal arcuate band and the proximal arcuate band, wherein these display screens are attached to the elastic member; and (e) one or more biometric sensors which are configured to collect data concerning arm tissue. In various examples, a ring, strap, bracelet, or bangle can be substituted for a band.

In an example, an arcuate band can undulate laterally as it spans the circumference a person's arm. In an example, distal and proximal arcuate bands can curve away from each other as they span a central portion of the anterior (upper) surface of a person's arm and can curve back toward each other as they span a side surface of the person's arm. In an example, an arcuate band can be attached to a person's arm by connecting two ends of the arcuate band with a clasp, clip, buckle, hook, pin, plug, or hook-and-eye mechanism. In an example, an arcuate band can be attached to a person's arm by stretching and sliding it over the person's hand onto the arm. In an example, an arcuate band can be attached to a person's arm by applying force to pull two ends apart to slip the member over the arm, wherein the two ends retract back towards each other when the force is removed.

In an example, an elastic member can be made from elastic fabric. In an example, an elastic member can be an elastic mesh. In an example, an elastic member can have four arcuate sides: two convex sides and two concave sides. In an example, one concave side can connect to the distal arcuate band and the other concave side can connect to the proximal band. In an example, two convex sides can be between the two bands. In an example, an elastic member can completely surround the perimeters of two display screens. In an example, an elastic member can flexibly-suspend two display screens between two arcuate bands. In an example, a display screen can be circular. In an example, the centers of two display screens can be connected to form a virtual line which is parallel to the longitudinal axis of an arm. In an example, the centers of two display screens can be connected to form a virtual line which is parallel to a line which is perpendicular to the circumferences of distal and proximal arcuate bands.

In an example, biometric sensors can be part of (or attached to) display screens and/or enclosures which house display screens. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 36 include: distal arcuate band 3601; proximal arcuate band 3602; elastic member 3603 between the two arcuate bands; display screens 3604 and 3605 suspended by the elastic member; and biometric sensors 3606 and 3607.

I claim:

1. An integrated system for managing cardiac rhythm including both a wearable device and an implanted device, wherein this system comprises:
a wearable device which is configured to be worn by a person, wherein the wearable device further comprises a light emitter which is configured to emit light toward the person's body tissue, a light receiver which is configured to receive light from the light emitter after the light has passed through and/or been reflected from the person's body tissue, and a wireless data transmitter; and
a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver;
wherein differences between the spectral distribution of light emitted from the light emitter and the spectral distribution of light received by the light receiver are analyzed in order to measure the amount of an analyte in the person's body tissue; and
wherein the operation of the cardiac rhythm management device is changed based on the amount of the analyte in the person's body tissue.

2. The system in claim 1 wherein the wearable device is a finger ring.

3. The system in claim 1 wherein the wearable device is a wrist band and/or arm band.

4. The system in claim 1 wherein the wearable device is a smart watch.

5. The system in claim 1 wherein the wearable device is an ear ring or other ear-worn device.

6. The system in claim 1 wherein the analyte is oxygen.

7. The system in claim 1 wherein the analyte is lactate or lactic acid.

8. The system in claim 1 wherein the implanted cardiac rhythm management device is a pacemaker.

9. The system in claim 1 wherein the system increases the frequency of electromagnetic pulses delivered to the person's heart when analysis of data from the light receiver indicates a low analyte level in body tissue.

10. The system in claim 1 wherein the system increases the magnitude of electromagnetic pulses delivered to the person's heart when analysis of data from the light receiver indicates a low analyte level in body tissue.

11. The system in claim 1 wherein there is a flexible light barrier between the light emitter and the light receiver.

12. The system in claim 1 wherein there is a compressible light barrier between the light emitter and the light receiver.

13. The system in claim 1 wherein there is a fluid-filled or gas-filled light barrier between the light emitter and the light receiver.

14. The system in claim 1 wherein the light emitter and the light receiver are on the same circumferential line of a wearable device, but at different radial locations around this circumference.

15. The system in claim 1 wherein the light emitter and the light receiver are on the same radial location around a wearable device, but on different circumferential lines.

16. The system in claim 1 wherein there are two or more light emitters and one light receiver on the wearable device.

17. The system in claim 1 wherein there is one light emitter and two or more light receivers on the wearable device.

18. The system in claim 1 wherein there is a plurality of pairs of light emitters and light receivers around the circumference of the wearable device.

19. The system in claim 1 wherein the light emitter emits infrared or near-infrared light.

20. The system in claim 1 wherein the light emitter emits light with frequency and/or spectrum changes over time.
